SEQUENCE LISTING (111 sequences)

SEQ ID NO: 1 - protein, 215 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 447 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 447 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 320 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGLVPRGSMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGG

SEQ ID NO: 5 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 7 - protein, 453 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 8 - protein, 453 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 9 - protein, 213 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 10 - protein, 213 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 208 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 12 - protein, 206 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPKTHMTHHAVSDHEATLRCWALSFYPAEITLTWQRDGEDQTQDTELVETRPAGDGTFQKWAAVVVPSGQEQRYTCHVQHEGLPKPLTLRWELSS

SEQ ID NO: 13 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGRTDAPKTHMTHHAVSDHEATLRCWALSFYPAEITLTWQRDGEDQTQDTELVETRPAGDGTFQKWAAVVVPSGQEQRYTCHVQHEGLPKPLTLRWELSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 14 - protein, 453 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 15 - protein, 455 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTNLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 16 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTSSASLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTS

SEQ ID NO: 17 - protein, 394 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGSRRELTDTLQAETDQLEDEKSALQTEIANLLKEKEKLEFILADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 18 - protein, 154 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAASKLERIARLEEKVKTLKAQNSELASTANMLREQVAQLKQKVM

SEQ ID NO: 19 - protein, 465 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSTRDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 20 - protein, 228 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKQLLFNKTKSVEFTFGNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWSTR

SEQ ID NO: 21 - protein, 174 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAASGGGGSGGGGSGGGGSGGGGTSTNNNEEEKSRLLEKENRELEKIIAEKEERVSELRHQLQSR

SEQ ID NO: 22 - protein, 414 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGSGGGGSGGGGSGGGGSGGGGTSVNQASTSRLEGLQSENHHLRMKITELDKDLEEVTMQLQDTDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 23 - protein, 193 aa, synthetic construct
MGWSCIILFLVATATGVHSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAASGGGGSGGGGSGGGGSGGGGTSVNQASTSRLEGLQSENHHLRMKITELDKDLEEVTMQLQDT

SEQ ID NO: 24 - protein, 414 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGSGGGGSGGGGSGGGGSGGGGTSTNNNEEEKSRLLEKENRELEKIIAEKEERVSELRHQLQSRDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 25 - protein, 213 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAQVKPEAWLSHGPSPGPGHLQLVCHVSGFYPKPVWVMWMRGEQEQQGTQRGDILPSADGTWYLRATLEVAAGEAADLSCRVKHSSLEGQDIVLYWEGSLVPRG

SEQ ID NO: 26 - protein, 223 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAQVKPEAWLSSGPSPGPGRLQLVCHVSGFYPKPVWVMWMRGEQEQQGTQLGDILPNANWTWYLRATLDVADGEAAGLSCRVKHSSLEGQDIILYWRGSGLNDIFEAQKIEWHE

SEQ ID NO: 27 - protein, 454 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGQVKPEAWLSHGPSPGPGHLQLVCHVSGFYPKPVWVMWMRGEQEQQGTQRGDILPSADGTWYLRATLEVAAGEAADLSCRVKHSSLEGQDIVLYWEGSLVPRGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 28 - protein, 489 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGSGGGGSGGGGSGGGGSGGGGTLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 29 - protein, 247 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAASGGGGSGGGGSGGGGSGGGGTPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLP

SEQ ID NO: 30 - protein, 489 aa, synthetic construct; identical to SEQ ID NO: 28

SEQ ID NO: 31 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 454 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 33 - protein, 212 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 34 - protein, 476 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRASDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 35 - protein, 202 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKPDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 36 - protein, 457 aa, synthetic construct
EVQLVESGGGLVQPGGSLRLSCAASGFPFNTFAMNWVRQAPGKGLEWLARIRSKSNNYATYYADSVKDRFTISRDNAKRSLYLQMNSLRAEDTAVYYCVRGKENNYGYAMDYWGQGTTVTVSSASTKGQVKPEAWLSHGPSPGPGHLQLVCHVSGFYPKPVWVMWMRGEQEQQGTQRGDILPSADGTWYLRATLEVAAGEAADLSCRVKHSSLEGQDIVLYWEGSLVPRGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 37 - protein, 213 aa, synthetic construct
DIVMTQSPLSLPVTPGEPASISCRSSKSLLHSNANTYLYWFLQKPGQSPQLLIYRMSNLASGVPDRFSGSGSGTDFTLKISRVEAEDVGVYYCMQHVEYPFTFGGGTKLEIKRTIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 38 - protein, 455 aa, synthetic construct
QVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSASTKGQVKPEAWLSHGPSPGPGHLQLVCHVSGFYPKPVWVMWMRGEQEQQGTQRGDILPSADGTWYLRATLEVAAGEAADLSCRVKHSSLEGQDIVLYWEGSLVPRGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 39 - protein, 455 aa, synthetic construct
QVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 40 - protein, 479 aa, synthetic construct
QVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRASDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 41 - protein, 207 aa, synthetic construct
QIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRTIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 42 - protein, 211 aa, synthetic construct
QIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 43 - protein, 201 aa, synthetic construct
QIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKPDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 44 - protein, 214 aa, synthetic construct
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 446 aa, synthetic construct
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 214 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 452 aa, synthetic construct
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 99 aa, synthetic construct
RTQAPKTHMTHHAVSDHEATLRCWALSFYPAEITLTWQRDGEDQTQDTELVETRPAGDGTFQKWAAVVVPSGQEQRYTCHVQHEGLPKPLTLRWEPLSS

SEQ ID NO: 49 - protein, 99 aa, synthetic construct [sequence omitted]

SEQ ID NO: 50 - protein, 102 aa, synthetic construct
QVKPEAWLSHGPSPGPGHLQLVCHVSGFYPKPVWVMWMRGEQEQQGTQRGDILPSADGTWYLRATLEVAAGEAADLSCRVKHSSLEGQDIVLYWEGSLVPRG

SEQ ID NO: 51 - protein, 112 aa, synthetic construct
QVKPEAWLSSGPSPGPGRLQLVCHVSGFYPKPVWVMWMRGEQEQQGTQLGDILPNANWTWYLRATLDVADGEAAGLSCRVKHSSLEGQDIILYWRGSGLNDIFEAQKIEWHE

SEQ ID NO: 52 - protein, 41 aa, synthetic construct
STNNNEEEKSRLLEKENRELEKIIAEKEERVSELRHQLQSR

SEQ ID NO: 53 - protein, 41 aa, synthetic construct
SVNQASTSRLEGLQSENHHLRMKITELDKDLEEVTMQLQDT

SEQ ID NO: 54 - protein, 41 aa, synthetic construct
RRELTDTLQAETDQLEDEKSALQTEIANLLKEKEKLEFILA

SEQ ID NO: 55 - protein, 41 aa, synthetic construct
KLERIARLEEKVKTLKAQNSELASTANMLREQVAQLKQKVM

SEQ ID NO: 56 - protein, 98 aa, synthetic construct
MSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGG

SEQ ID NO: 57 - protein, 217 aa, synthetic construct
DIVMTQSPLSLPVTPGEPASISCRSSKSLLHSNANTYLYWFLQKPGQSPQLLIYRMSNLASGVPDRFSGSGSGTDFTLKISRVEAEDVGVYYCMQHVEYPFTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 58 - protein, 457 aa, synthetic construct
EVQLVESGGGLVQPGGSLRLSCAASGFPFNTFAMNWVRQAPGKGLEWLARIRSKSNNYATYYADSVKDRFTISRDNAKRSLYLQMNSLRAEDTAVYYCVRGKENNYGYAMDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 59 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGQVKPEAWLSSCPSPGPGRLQLVCHVSGFYPKPVWVMWMRGEQEQQGTQLSEILPNANWTWYLRATLDVADGEAAGLSCRVKHSSLEGQDIILYWRGSGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 60 - protein, 214 aa, synthetic construct
EIVMTQSPATLSVSPGERATLSCRASQSIGNNLHWYQQKPGQAPRLLIYYASQSISGIPARFSGSGSGTEFTLTISSLQSEDFAVYYCQQSNSWPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 443 aa, synthetic construct
QVQLQESGPGLMKPSETLSLTCSVSGDSIRSDYWSWIRQPPGKGLEYIGYVSYSGSTYYNPSLKSRVTISVDTSKNRFSLKLNSVTAADTAVYYCARWDGDYWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 479 aa, synthetic construct
EVQLVESGGGLVQPGGSLRLSCAASGFPFNTFAMNWVRQAPGKGLEWLARIRSKSNNYATYYADSVKDRFTISRDNAKRSLYLQMNSLRAEDTAVYYCVRGKENNYGYAMDYWGQGTTVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRASDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 63 - protein, 447 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 447 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 208 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 66 - protein, 454 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGQVKPEAWLSHGPSPGPGHLQLVCHVSGFYPKPVWVMWMRGEQEQQGTQRGDILPSACGTWYLRATLEVAAGEAADLSCRVKHSSLEGQDIVLYWEGSLVPRGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 67 - protein, 454 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGQVKPEAWLSHGPSPGPGHLQLVCHVSGFYPKPVWVMWMRGEQEQQGTQRGDILPCADGTWYLRATLEVAAGEAADLSCRVKHSSLEGQDIVLYWEGSLVPRGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 68 - protein, 454 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGQVKPEAWLSHGPSPGPGHLQLVCHVSGFYPKPVWVMWMRGEQEQQGTQRGCILPSADGTWYLRATLEVAAGEAADLSCRVKHSSLEGQDIVLYWEGSLVPRGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 69 - protein, 208 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTIQRTPKICVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 70 - protein, 208 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTIQRTPKIQVYSRHCAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 71 - protein, 208 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDC

SEQ ID NO: 72 - protein, 454 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGQVKPEACLSHGPSPGPGHLQLVCHVSGFYPKPVWVMWMRGEQEQQGTQRGDILPSADGTWYLRATLEVAAGEAADLSCRVKHSSLEGQDIVLYWEGSLVPRGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 73 - protein, 454 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGQVKPEAWLSHCPSPGPGHLQLVCHVSGFYPKPVWVMWMRGEQEQQGTQRGDILPSADGTWYLRATLEVAAGEAADLSCRVKHSSLEGQDIVLYWEGSLVPRGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 74 - protein, 209 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMC

SEQ ID NO: 75 - protein, 209 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTIQRTPKIKVYSRHPAENGKSNFLNCYVSGFHPSDIEVDLLKNGERIEKVEHSDLSFSKDWSFYLLYYTEFTPTEKDEYACRVNHVTLSQPKIVKWDRDMC

SEQ ID NO: 76 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGQVKPEAWLSHCPSPGPGHLQLVCHVSGFYPKPVWVMWMRGEQEQQGTQRGDILPSADGTWYLRATLEVAAGEAADLSCRVKHSSLEGQDIVLYWEGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 77 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGQVKPEAWLSHCPSPGPGHLQLVCHVSGFYPKPVWVMWMRGEQEQQGTQRSDILPSADGTWYLRATLEVAAGEAADLSCRVKHSSLEGQDIVLYWEGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 78 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGQVKPEAWLSHCPSPGPGHLQLVCHVSGFYPKPVWVMWMRGEQEQQGTQRSEILPSADGTWYLRATLEVAAGEAADLSCRVKHSSLEGQDIVLYWEGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 79 - protein, 464 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGQVKPEAWLSSCPSPGPGRLQLVCHVSGFYPKPVWVMWMRGEQEQQGTQLGDILPNANWTWYLRATLDVADGEAAGLSCRVKHSSLEGQDIILYWRGSGLNDIFEAQKIEWHEDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 80 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGQVKPEAWLSSCPSPGPGRLQLVCHVSGFYPKPVWVMWMRGEQEQQGTQLGDILPNANWTWYLRATLDVADGEAAGLSCRVKHSSLEGQDIILYWRGSGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 81 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGQVKPEAWLSSCPSPGPGRLQLVCHVSGFYPKPVWVMWMRGEQEQQGTQLSDILPNANWTWYLRATLDVADGEAAGLSCRVKHSSLEGQDIILYWRGSGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 82 - protein, 459 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQQASSMCVPDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 83 - protein, 221 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQQASSMCVP

SEQ ID NO: 84 - protein, 459 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAIAELPPKVSVFVPPCDGFFGNPRKSKLYCQATGFSPRQIQVSWLREGKQVGSGVTTDQVQAEAKESGPTTYKVYSTLTIKESDWLGQSMFTCRVDHRGLTFQQQASSMSVPDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 85 - protein, 221 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVIAELPPKVSVFVPPCDGFFGNPRKSKLYCQATGFSPRQIQVSWLREGKQVGSGVTTDQVQAEAKESGPTTYKVYSTLTIKESDWLGQSMFTCRVDHRGLTFQQQASSMSVP

SEQ ID NO: 86 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGQVKPEAWLSHCPSPGPGHLQLVCHVSGFYPKPVWVMWMRGEQEQQGTQRGDILPSADGTWYLRATLEVAAGEAADLSCRVKHSSLEGQDIVLYWEGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 87 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGQVKPEAWLSHCPSPGPGHLQLVCHVSGFYPKPVWVMWMRGEQEQQGTQRSEILPSADGTWYLRATLEVAAGEAADLSCRVKHSSLEGQDIVLYWEGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 88 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGQVKPEAWLSSCPSPGPGRLQLVCHVSGFYPKPVWVMWMRGEQEQQGTQLGDILPNANWTWYLRATLDVADGEAAGLSCRVKHSSLEGQDIILYWRGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 89 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGQVKPEAWLSSCPSPGPGRLQLVCHVSGFYPKPVWVMWMRGEQEQQGTQLSEILPNANWTWYLRATLDVADGEAAGLSCRVKHSSLEGQDIILYWRGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 90 - protein, 111 aa, synthetic construct
IAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQQASSMCVP

SEQ ID NO: 91 - DNA, 333 bp, synthetic construct
  source 1..333: mol_type = other DNA
atcgccgagctgccccccaaggtgagcgtgttcgtgccccctagagacggcttcttcggcaaccctagaaagagcaagctgatctgccaagccaccggcttcagccctagacagatccaagtgagctggctgagagagggcaagcaagtgggcagcggcgtgacaaccgaccaagtgcaagccgaggccaaggagagcggccccaccacctacaaggtgacaagcaccctgaccatcaaggagagcgactggctggggcagagcatgttcacctgcagagtggaccacagaggcctgacctttcagcaacaagctagcagcatgtgcgtgccc

SEQ ID NO: 92 - DNA, 294 bp, synthetic construct
  source 1..294: mol_type = other DNA
agaacagacgcccctaagacacacatgacccaccacgccgtgtccgatcacgaagccacactgagatgttgggccctgagcttctaccccgccgagatcacactgacctggcagagagatggcgaggatcagacccaggataccgagctggtggaaacaagacctgccggcgacggcaccttccagaaatgggctgctgtggtggtgcctagcggccaagagcagagatacacctgtcacgtgcagcacgagggcctgcctaagcctctgactctgagatgggagctgagcagc

SEQ ID NO: 93 - DNA, 297 bp, synthetic construct
  source 1..297: mol_type = other DNA
atccagcggacccctaagatccaggtgtacagcagacaccccgccgagaacggcaagagcaacttcctgaactgctacgtgtccggctttcaccccagcgacattgaggtggacctgctgaagaacggcgagcggatcgagaaggtggaacacagcgatctgagcttcagcaaggactggtccttctacctgctgtactacaccgagttcacccctaccgagaaggacgagtacgcctgcagagtgaaccacgtgacactgagccagcctaagatcgtgaagtgggacagagacatg

SEQ ID NO: 94 - DNA, 306 bp, synthetic construct
  source 1..306: mol_type = other DNA
caagtgaaacctgaggcctggctttcccatggtccaagccctggccctggacatctgcagcttgtctgtcatgtcagtgggttctaccctaagccagtgtgggtgatgtggatgaggggtgagcaggagcagcagggcacccaacgaggagacatcctgccttctgctgatggcacctggtaccttcgggctaccttggaagtggcagccggagaggccgccgatcttagttgtcgggtcaagcattcatccctggaggggcaagacatcgtgttgtactgggaaggctctctggttcccagaggg

SEQ ID NO: 95 - DNA, 336 bp, synthetic construct
  source 1..336: mol_type = other DNA
caggtgaagcctgaggcttggctgagttccggaccaagccccggtccgggtcggctgcagctggtgtgccacgtgtcaggattctatcccaagcccgtatgggtcatgtggatgagaggagaacaagagcagcaagggacccagctcggcgacatcctgcccaatgcaaattggacatggtacctgcgcgcaactctggatgtggcggatggagaggccgccggactgtcttgccgagtgaaacattcttcactggagggccaggatatcatcctctactggcgcggttccggcctgaatgacatcttcgaggcccagaaaattgagtggcatgag

SEQ ID NO: 96 - DNA, 123 bp, synthetic construct
  source 1..123: mol_type = other DNA
agtacaaataacaacgaggaggagaagtctcggcttctcgagaaggaaaaccgggaactggagaagattattgcagagaaagaggagcgcgtttctgagctgcgccaccagctgcagagcaga

SEQ ID NO: 97 - DNA, 123 bp, synthetic construct
  source 1..123: mol_type = other DNA
tctgtgaatcaggcaagcactagcagacttgaaggactccagtccgagaatcaccacttgcgaatgaagatcaccgagttggataaggacctggaggaagtgacaatgcagctgcaggatacc

SEQ ID NO: 98 - DNA, 123 bp, synthetic construct
  source 1..123: mol_type = other DNA
aagctggaacggatcgcccggctggaagagaaagtgaaaaccctgaaggcccagaacagcgagctggcctctaccgccaacatgctgagagaacaggtggcccagctgaagcagaaagtcatg

SEQ ID NO: 99 - DNA, 123 bp, synthetic construct
  source 1..123: mol_type = other DNA
cggagagagctgaccgatacactgcaggccgagacagaccagctggaagatgagaagtctgccctgcagaccgagatcgccaacctgctgaaagagaaagagaagctcgagttcatcctggcc

SEQ ID NO: 100 - DNA, 294 bp, synthetic construct
  source 1..294: mol_type = other DNA
atgagcgacagcgaagtgaatcaagaggccaagcctgaagtgaaacccgaagtgaagcctgagacacacatcaacctgaaggtgtccgacggcagcagcgagatcttcttcaagatcaagaaaaccacacctctgcggcggctgatggaagccttcgccaaaagacagggcaaagagatggacagcctgcggttcctgtacgacggcatcagaatccaggccgatcagacccctgaggacctggacatggaagataacgacatcatcgaggcccacagagagcagatcggagga

SEQ ID NO: 101 - protein, 453 aa, synthetic construct
EVQLVESGGGLVQPGGSLRLSCAASGFPFNTFAMNWVRQAPGKGLEWLARIRSKSNNYATYYADSVKDRFTISRDNAKRSLYLQMNSLRAEDTAVYYCVRGKENNYGYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 219 aa, synthetic construct
DIVMTQSPLSLPVTPGEPASISCRSSKSLLHSNANTYLYWFLQKPGQSPQLLIYRMSNLASGVPDRFSGSGSGTDFTLKISRVEAEDVGVYYCMQHVEYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 443 aa, synthetic construct
QVQLQESGPGLMKPSETLSLTCSVSGDSIRSDYWSWIRQPPGKGLEYIGYVSYSGSTYYNPSLKSRVTISVDTSKNRFSLKLNSVTAADTAVYYCARWDGDYWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 452 aa, synthetic construct
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 214 aa, synthetic construct
DIVMTQSPLSLPVTPGEPASISCRSSKSLLHSNANTYLYWFLQKPGQSPQLLIYRMSNLASGVPDRFSGSGSGTDFTLKISRVEAEDVGVYYCMQHVEYPFTFGGGTKLEIKRTIQRTPKIKVYSRHPAENGKSNFLNCYVSGFHPSDIEVDLLKNGERIEKVEHSDLSFSKDWSFYLLYYTEFTPTEKDEYACRVNHVTLSQPKIVKWDRDMC

SEQ ID NO: 106 - protein, 452 aa, synthetic construct
EVQLVESGGGLVQPGGSLRLSCAASGFPFNTFAMNWVRQAPGKGLEWLARIRSKSNNYATYYADSVKDRFTISRDNAKRSLYLQMNSLRAEDTAVYYCVRGKENNYGYAMDYWGQGTTVTVSSASTKGQVKPEAWLSHCPSPGPGHLQLVCHVSGFYPKPVWVMWMRGEQEQQGTQRSEILPSADGTWYLRATLEVAAGEAADLSCRVKHSSLEGQDIVLYWEGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 107 - protein, 453 aa, synthetic construct
EVQLVESGGGLVQPGGSLRLSCAASGFPFNTFAMNWVRQAPGKGLEWLARIRSKSNNYATYYADSVKDRFTISRDNAKRSLYLQMNSLRAEDTAVYYCVRGKENNYGYAMDYWGQGTTVTVSSASTKGQVKPEAWLSSCPSPGPGRLQLVCHVSGFYPKPVWVMWMRGEQEQQGTQLSEILPNANWTWYLRATLDVADGEAAGLSCRVKHSSLEGQDIILYWRGSGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 108 - protein, 230 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGQVKPEAWLSHCPSPGPGHLQLVCHVSGFYPKPVWVMWMRGEQEQQGTQRGDILPSADGTWYLRATLEVAAGEAADLSCRVKHSSLEGQDIVLYWEGSHHHHHHHH

SEQ ID NO: 109 - protein, 230 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGQVKPEAWLSHCPSPGPGHLQLVCHVSGFYPKPVWVMWMRGEQEQQGTQRSDILPSADGTWYLRATLEVAAGEAADLSCRVKHSSLEGQDIVLYWEGSHHHHHHHH

SEQ ID NO: 110 - protein, 230 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGQVKPEAWLSHCPSPGPGHLQLVCHVSGFYPKPVWVMWMRGEQEQQGTQRSEILPSADGTWYLRATLEVAAGEAADLSCRVKHSSLEGQDIVLYWEGSHHHHHHHH

SEQ ID NO: 111 - protein, 231 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHHHH

(19) United States
(12) Patent Application Publication
(10) Pub. No.: US 2026/0209385 A1
(43) Pub. Date: Jul. 23, 2026

(54) Bispecific/multi-specific antibodies and uses thereof

(71) Applicant: ANALYTICAL BIOSCIENCES SHANGHAI LIMITED, Shanghai (CN)

(72) Inventors: Qianting Zhai, Shanghai (CN); Huan Lan, Gao'an (CN); Mingjian Fei, Shanghai (CN); Xiaodong Huang, Belmont, CA (US); Chengkun Wang, Beijing (CN)

(21) Appl. No.: 19/135,813

(22) PCT Filed: Nov. 22, 2023
(86) PCT No.: PCT/CN2023/133365

§ 371 Date: Jun. 5, 2025

(30) Foreign Application Priority Data
Dec. 8, 2022 (WO) PCT/CN2022/137489

(51) Int. Cl. C07K 16/32; C07K 16/28; C07K 16/40

(52) U.S. Cl. CPC C07K 16/32; C07K 16/2833; C07K 16/40; C07K 2317/31; C07K 2317/51; C07K 2317/522; C07K 2317/524; C07K 2317/526; C07K 2317/55; C07K 2317/94; C07K 2319/00

(57) ABSTRACT

Provided is a new protein with four chains. The new protein is a bispecific or multispecific protein. Further provided are an isolated polynucleotide encoding the heavy chain 1, 2, the light chain 1 or 2 of the new protein, a set of isolated polynucleotides comprising the polynucleotides encoding the heavy chain 1, 2, light chain 1 or 2 respectively, an isolated vector comprising the isolated polynucleotide, a host cell comprising the isolated polynucleotide or the set of isolated polynucleotides or the vector, a method of preparing the new protein, a pharmaceutical composition comprising the new protein, a method for production of a new protein with improved thermal stability, use of the new protein or the isolated polynucleotide or the vector or the host cell or the pharmaceutical composition in the manufacture of a drug for preventing or treating a disease, and a method of preventing or treating a disease.

### TECHNICAL FIELD

The present disclosure relates to a new protein comprising at least two antigen binding domains. Particularly, the present disclosure relates to a new protein with four chains, wherein this new protein is derived from an IgG-like or an IgG-based antibody and it is a bispecific or multi-specific protein. The present disclosure also relates to an isolated polynucleotide encoding the heavy chain 1, the light chain 1, the heavy chain 2 or the light chain 2 of the new protein, a set of isolated polynucleotides, comprising the polynucleotide encoding the heavy chain 1, the polynucleotide encoding light chain 1, the polynucleotide encoding heavy chain 2 and the polynucleotide encoding light chain 2 of the new protein, an isolated vector comprising the isolated polynucleotide, a host cell comprising the isolated polynucleotide or the set of isolated polynucleotides or the isolated vector, a method for production of a new protein with an adjusted thermal stability, a method of preparing the new protein, a pharmaceutical composition comprising the new protein or the isolated polynucleotide or the set of isolated polynucleotides, or the isolated vector or the host cell, and a pharmaceutically acceptable carrier, use of the new protein or the isolated polynucleotide or the isolated vector or the host cell or the pharmaceutical composition in the manufacture of a drug for preventing or treating a disease, or in the manufacture of a kit for diagnosing a disease, and a method of preventing or treating a disease in a subject in need thereof, comprising administrating to the subject a therapeutically effective amount of the new protein or the isolated polynucleotide or the set of isolated polynucleotides or the isolated vector or the host cell or the pharmaceutical composition.

### BACKGROUND

The statements in this section provide background information related to the present disclosure and do not necessarily constitute prior art.

Bispecific/multi-specific antibodies are rapidly growing class of therapeutics. Bispecific antibodies (BsAb) can bind to two different antigens or two different epitopes of the same antigen to improve the specificity and therapeutic potential. Multi-specific antibodies (MsAb) can bind to more than two different antigens or two epitopes of the same antigen to improve the specificity and therapeutic potential. BsAb/MsAb can be constructed in many different configurations, such as CrossMab, DVD-Tg, scFv-Fc dimer; DART, and the like.

Several techniques were developed to enable heterodimerization of two distinct heavy chains to suppress the formation of heavy chain homodimers, such as knobs-into-holes (KIH) (Ridgway et al., 1996), electrostatic steering of CH3 (Gunasekaran et al., 2010), the SEED technology (Davis et al., 2010), the DuoBody (Labrijn et al., 2013), the Azymetric platform (Escobar-Cabrera et al., 2017), and the XmAb bispecific platform (Moore et al., 2019).

However, the selective pairing of light-heavy chains of each antibody remains challenging. There have been several approaches to prevent light chain mispairing. The IgG-like bispecific can be produced by assembling half-bodies cultured from two transformed E. coli cell lines (Spiess et al., 2013). But the BsAbs expressed within a single cell line are preferred. Another option to bypass this issue is the use of a common light chain combined with two different heavy chains. However, identifying a common light chain needs other technologies including transgenic animals with a single light chain, or extensive engineering of the complementarity-determining regions (Kitazawa and Shima, 2020). Several other strategies have also been developed to overcome the light-heavy chain pairing problem, such as the switched domain of CH1/CL in Roche's CrossMab Platform (Regula et al., 2018), and the engineered CH1/CL interface with additional mutations in the variable domains (Dillon et al., 2017; Lewis et al., 2014; Liu et al., 2015; Zhao et al., 2021). Most of the above strategies require chain ratio optimization to
maximize the correct LC/HC assembly. Therefore, there is a need to design a bi-specific/multi-specific format with desirable selective light-heavy chain pairing to improve their expression, stability, and purity.

### SUMMARY

For the purpose mentioned above, provided herein is a new protein comprising two pairs of polypeptides, wherein each pair of polypeptides consists of a heavy chain and a light chain, the heavy chain comprises a VH domain and a CHi domain, the light chain comprises a VL domain and a CL domain, and the VH domain and the VL domain of a first pair of polypeptides can form a first Fv fragment that specifically binds to a first antigen, the VH domain and the VL domain of a second pair of polypeptides can form a second Fv fragment that specifically binds to a second antigen, and wherein at least one pair of dimerization domains are introduced into the first pair of polypeptides, and each pair of dimerization domains can form a dimer themselves alone or in conjunction with other proteins, wherein the dimerization domains are homo/heterodimerization domains.

In some embodiments, the first and the second antigens are different antigens. In some embodiments, the first and the second antigens are different epitopes of the same antigen.

In some embodiments, each pair of polypeptides consists of a heavy chain and a light chain. The heavy chain comprises a VH domain, a CH1 domain, a CH2 domain and a CH3 domain, resulting in a long chain of VH-CH1-CH2-CH3. The light chain comprises a VL domain and a CL domain, resulting in a short chain of VL-CL.

In some embodiments, the new protein further contains one or more modifications, wherein the modifications can improve the correct assembly and thermal stability of the new protein. In some embodiments, the modification is located in CH3 domains, such as knobs-into-holes (KIH) (Ridgway et al., 1996), electrostatic steering of CH3 (Gunasekaran et al., 2010), the SEED technology (Davis et al., 2010), the XmAb bispecific platform (Moore et al., 2019), the Azymetric platform (Escobar-Cabrera et al., 2017), and the DuoBody (Labrijn et al., 2013).

In some embodiments, this new protein is derived from IgG type antibody and it is a bispecific or multi-specific protein. Particularly, provided herein is a new protein having four chains. The four chains are named heavy chain 1 (H chain 1), light chain 1 (L chain 1), heavy chain 2 (H chain 2) and light chain 2 (L chain 2), respectively. In some embodiments, the new protein comprises four chains of polypeptides, the four chains of polypeptides are heavy chain 1, light chain 1, heavy chain 2 and light chain 2,
wherein the heavy chain 1 and the heavy chain 2 comprise a VH domain, a CH1 domain, a CH2 domain and a CH3 domain and form a long chain of VH-CH1-CH2-CH3, respectively; the light chain 1 and the light chain 2 comprise a VL domain and a CL domain and form a short chain of VL-CL respectively,
wherein the heavy chain 1 and the light chain 1 are derived from an antibody A, the single heavy chain 1 pairs with the single light chain 1 and forms half-body A; the heavy chain 2 and the light chain 2 are derived from an antibody B, the single heavy chain 2 pairs with the single light chain 2 and forms half-body B; and
wherein the CHI domain of the heavy chain 1 pairs with the CL domain of the light chain 1, named herein as a combination of CH1/CL in half-body A, and the CH1 domain of the heavy chain 2 pairs with the CL domain of the light chain 2, named herein as a combination of CH1/CL in half-body B.

In some embodiments, the combination CH1/CL in half-body A is replaced by dimerization domains, including homodimers and heterodimers. Each pair of the homodimerization or heterodimerization domains can form a dimer themselves alone or in conjugation with other proteins.

In some embodiments, at least one pair of dimerization domains are introduced to replace the CH1/CL in half-body A and/or the CH1/CL in half-body B.

“Homodimerization domain” as used herein refers to a domain that can form a homodimer itself alone or in conjugation with other proteins. The homodimerization domains can be derived from human or other mammals. They are used to replace the CH1/CL in half-body A or in half-body B.

In some embodiments, the homodimerization domains can be derived from human or other mammals. The homodimerization domains are selected from IgG CH3/CH3 domain, IgM CH2/CH2 domain or IgE CH2/CH2 domain. In some embodiments, each homodimerization domain bears different mutations for better dimerization.

In some embodiments, only the combination CH1/CL in half-body A is replaced with homodimerization domains or modified homodimerization domains. Meanwhile, the CH1/CL in half-body B is unchanged.

In some embodiments, the combination CH1/CL in half-body A is replaced with homodimerization domains or modified homodimerization domains. Meanwhile, the combination CH1/CL in half-body B is also replaced with homodimerization domains or modified homodimerization domains.

Wherein, “CH1/CL in half-body A is replaced with CH3^{IgG}/CH3^{IgG}” means the CH1 domain in half-body A is replaced with the WT CH3 domain of IgG1 and the CL domain is replaced with the WT CH3 domain of IgG1, i.e., the CH1 domain of heavy chain 1 is replaced with the WT CH3 domain of IgG1 and the CL domain of light chain 1 is replaced with WT CH3 domain of IgG1.

Wherein, “CH1/CL in half-body A is replaced with CH2^{IgM}/CH2^{IgM}” means the CH1 domain in half-body A is replaced with the WT CH2 domain of IgM and the CL domain in half-body A is replaced with the WT CH2 domain of IgM, i.e., the CH1 domain of heavy chain 1 is replaced with the WT CH2 domain of IgM and the CL domain of light chain 1 is replaced with WT CH2 domain of IgM.

Wherein, “CH1/CL in half-body A is replaced with knob^{IgGCH3}/hole^{IgGCH3}” means the CH1 domain in half-body A is replaced with the knob form of IgG1 CH3 domain and the CL domain in half-body A is replaced with the hole form of IgG1 CH3 domain, i.e., the CH1 domain of heavy chain 1 is replaced with the knob form of IgG1 CH3 domain and the CL domain of light chain 1 is replaced with the hole form of IgG1 CH3 domain.

Wherein, “CH1/CL in half-body A is replaced with hole^{IgGCH3}/knob^{IgGCH3}” means the CH1 domain in half-body A is replaced with the hole form of IgG1 CH3 domain and the CL domain is replaced with the knob form of IgG1 CH3 domain, i.e., the CH1 domain of heavy chain 1 is replaced with the hole form of IgG1 CH3 domain and the CL domain of light chain 1 is replaced with the knob form of IgG1 CH3 domain.

Wherein, “CH1/CL in half-body A is replaced with knob^{IgMCH2}/hole^{IgMCH2}” means the CH1 domain in half-body A is replaced with the knob form of IgM CH2 domain and the CL domain is replaced with the hole form of IgM CH2 domain, i.e., the CH1 domain of heavy chain 1 is replaced with the knob form of IgM CH2 domain and the CL domain of light chain 1 is replaced with the hole form of IgM CH2 domain.

Wherein, “CH1/CL in half-body A is replaced with hole^{IgMCH2}/knob^{IgGCH2}” means the CH1 domain of half body A is replaced with the hole form of IgM CH2 domain and the CL domain is replaced with the knob form of IgM CH2 domain, i.e., the CH1 domain of heavy chain 1 is replaced with the hole form of IgM CH2 domain and the CL domain of light chain 1 is replaced with the knob form of IgM CH2 domain.

“Heterodimerization domain” as used herein refers to domains that can form heterodimer themselves alone or in conjunction with other proteins. They are used to replace the CH1/CL in half-body A. The heterodimerization domains can be derived from human or other mammals.

In some embodiments, at least one pair of the heterodimerization domains can be derived from human or other mammals, and the heterodimerization domains are not TCR α/β or TCR β/α constant domains, preferably the heterodimerization domains are not TCR α/β or TCR β/α constant domains or HLA/β2m or β2m/HLA domains.

In some embodiments, only the combination CH1/CL in half-body A is replaced with other heterodimerization domains. Meanwhile, the half-body B is unchanged.

In some embodiments, the combination CH1/CL in half-body A is replaced with other heterodimerization domains. Meanwhile, the combination CH1/CL in half-body B is also replaced with other heterodimerization domains.

In some embodiments, the heterodimerization domains are selected from one of the combinations of IgG like domain HLA/β2m, β2m/HLA, CD1s/β2m and β2m/CD1s (for example, CD1a/β2m, β2m/CD1a, CD1b/β2m and β2m/CD1b), or the heterodimerization domains are selected from one of the combinations of non-IgG-like GABA1/GABA2, GABA2/GABA1, SIRPα/CD47, or CD47/SIRPα.

Wherein, “CH1/CL in half-body A is replaced with HLA/β2m” means the CH1 domain of half-body A is replaced with the α3 domain of HLA and CL is replaced with β2m, i.e., the CH1 domain of heavy chain 1 is replaced with the α3 domain of HLA and CL of light chain 1 is replaced with β2m;

“CH1/CL of half-body A is replaced with β2m/HLA” means CH1 of half-body A is replaced with β2m and CL is replaced with the α3 domain of HLA, i.e., the CH1 domain of heavy chain 1 is replaced with β2m and CL of light chain 1 is replaced with HLA;

“CH1/CL of half-body A is replaced with CD1s/β2m” means CH1 of half-body A is replaced with the α3 domain of CD1s and CL is replaced with β2m, i.e., the CH1 domain of heavy chain 1 is replaced with the α3 domain of CD1s and CL of light chain 1 is replaced with β2m;

“CH1/CL of half-body A is replaced with β2m/CD1s” means CH1 of half-body A is replaced with β2m and CL is replaced with the α3 domain of CDIs, i.e., the CH1 domain of heavy chain 1 is replaced with β2m and CL of light chain 1 is replaced with the α3 domain of CD1s;

“CH1/CL of half-body A is replaced with GABA1/GABA2” means CH1 of half-body A is replaced with GABA1 and CL is replaced with GABA2, i.e., the CH1 domain of heavy chain 1 is replaced with GABA1 and CL of light chain 1 is replaced with GABA2;

“CH1/CL of half-body A is replaced with GABA2/GABA1” means CH1 of half-body A is replaced with GABA2 and CL is replaced with GABA1, i.e., the CH1 domain of heavy chain 1 is replaced with GABA2 and CL of light chain 1 is replaced with GABA1;

“CH1/CL of half-body A is replaced with SIRPα/CD47” means CH1 of half-body A is replaced with SIRPα and CL is replaced with CD47, i.e., the CH1 domain of heavy chain 1 is replaced with SIRPα and CL of light chain 1 is replaced with CD47;

“CH1/CL of half-body A is replaced with CD47/SIRPα” means CH1 of half-body A is replaced with CD47 and CL is replaced with SIRPα, i.e., the CH1 domain of heavy chain 1 is replaced with CD47 and CL of light chain 1 is replaced with SIRPα.

“HLA” as used herein refers to the α3 domain of human leukocyte antigen (HLA) gene locus encoded MHC-I protein. The α3 domain of allotype A*02:01 (aa: 182-276+LSS in heavy chain 1 or aa: 185-276+LSS in light chain 1) was used as an example in this patent, and it is shown in SEQ ID NO: 48.

“β2m” as used herein refers to human β2-microglobulin (aa: 1-99), which can bind to the α3 domain of MHC-I or MHC-I like protein, forming a non-covalent bond heterodimer. The protein sequence of “β2m” is shown in SEQ ID NO: 49.

“Human CD1s” or “CD1s”, as used herein, means the human CD1 molecules that present lipids and glycolipids on the cell surface for T-cell recognition. In some embodiments, the CD1s include CD1a, CD1b, CD1c and CD1d. In some embodiments, CD1s refer to CD1a or CD1b.

“CD1a”, as used herein, refers to the α3 domain (aa: 184-278, counting from the 18th amino acid, derived from PDB 7KPI, SEQ ID NO: 50) of human CD1a protein, which can bind to other protein domains, forming a heterodimer with covalent bonds or non-covalent bonds.

“CD1b”, as used herein, refers to the α3 domain (aa: 184-278, counting from the 18th amino acid, derived from PBD 6D64, SEQ ID NO: 51) of human CD1b protein, which can bind to other protein domains, forming a heterodimer with covalent bonds or non-covalent bonds.

“GABA1” as used herein refers to Gamma-aminobutyric acid receptor subunit alpha-1 (aa: 878-919) which can bind to other protein domains, forming a heterodimer with covalent bonds or non-covalent bonds, and it is shown in SEQ ID NO: 52.

“GABA2” as used herein refers to R797H natural variant of gamma-aminobutyric acid type B receptor subunit 2 (aa: 779-819), which can bind to other protein domains, forming a heterodimer and it is shown in SEQ ID NO: 53.

The term “domain” as used herein refers to a part of a molecule or structure that shares common physical, chemical or structural characteristics, such as similar hydrophobic or polar properties. The exemplary domain may include a protein binding domain, a DNA binding domain, an ATP binding domain, or a similar folding structure such as globular or helical features. The domains can be identified according to their homology with conserved structural or functional motifs.

The term “protein” is defined as a biological polymer comprising units derived from amino acids linked by peptide bonds. A protein can be composed of one or more chains.

The terms “pharmaceutically acceptable”, or “physiologically tolerable” and the grammatical variations thereof, as used herein, which refer to compositions, vehicles, diluents, and reagents, are used interchangeably and represent that the materials are capable of administration in or on a human being without the production of undesirable physiological effects that prevent its therapeutic use, such as nausea, dizziness, gastric discomfort and the like.

“Cancer” “tumor” or “neoplasia” are used as synonymous terms and refer to any of several diseases that are characterized by an uncontrolled and abnormal proliferation of cells, the ability of the affected cells to spread locally or through the bloodstream and the lymphatic system to other parts of the body (metastasize), as well as any of several characteristic structural and/or molecular features. A “cancerous tumor” or “malignant cell” is understood as a cell with specific structural properties, which include reduced differentiation and the increased capacity for invasion and metastasis. Examples of cancers that can be treated using antibodies of the invention include solid tumors and hematologic cancers. Additional examples of cancers that can be treated using the antibody of the invention include breast, lung, brain, bone, liver, kidney, colon, head and neck, ovarian, hematopoietic (e.g., leukemia) and prostate cancers. Additional examples of cancers that can be treated using multivalent and multi-specific antibodies include, but are not limited to, carcinoma, lymphoma, blastoma, sarcoma, and leukemia. Among the most particular examples of such cancers include squamous cell cancer, small cell lung cancer, non-small cell lung cancer, lung adenocarcinoma, squamous cell carcinoma of the lung, peritoneal cancer, hepatocellular cancer, gastrointestinal cancer, pancreatic cancer, glioblastoma, cancer cervical cancer, ovarian cancer, liver cancer, bladder cancer, hepatoma, breast cancer, colon cancer, colorectal cancer, endometrial or uterine carcinoma, carcinoma of the salivary glands, kidney cancer, liver cancer, prostate cancer, vulvar cancer, thyroid cancer, hepatic carcinoma and various types of cancers of the head and neck. Other cancers and tumors that can be treated using multivalent and multispecific antibodies are described herein or are otherwise known in the art.

An “effective amount” of an antibody, as disclosed herein, is an amount sufficient to achieve a specifically indicated purpose in order to cause an observable change in the level of one or more biological activities related to the target cell to which the antibody binds. The change can increase the activity level of the target. The change can reduce the activity level of the target. An “effective amount” can be determined empirically and in a routine manner, in relation to the stated purpose.

The term “therapeutically effective amount” refers to an amount of an antibody, another multivalent and multispecific drug of the invention or another drug effective to “treat” a disease or disorder in a patient or mammal. In the case of cancer, the therapeutically effective amount of the drug can reduce angiogenesis and neovascularization; reduce the number of cancer cells; reduce the size of the tumor; inhibit (i.e., slow down to some extent or stop) the infiltration of cancer cells into peripheral organs; inhibit (i.e., slow down to a certain point or stop) tumor metastasis; inhibit, to some extent, tumor growth or the incidence of tumors; stimulate immune responses against cancer cells and/or alleviate to some extent one or more of the symptoms associated with cancer. See the definition in this document of “treat.” A “therapeutically effective amount” may also refer to an effective amount, at the dosages and for the periods of time necessary, to achieve a desired therapeutic result. A therapeutically effective amount of a composition of the invention may vary depending on factors such as the pathology, age, sex and weight of the individual and the ability of the composition to elicit a desired response in the individual. A therapeutically effective amount is also one in which the toxic or detrimental effects of the therapeutic composition are overcome by the therapeutically beneficial effects.

Antibodies that can be used in multivalent and multispecific antibodies include, but are not limited to, monoclonal, multispecific, human, humanized, primatized and chimeric antibodies. The immunoglobulin or antibody molecules of the invention can be of any type (for example, IgG, IgE, IgM, IgD, IgA and IgY), class (for example, IgG1, IgG2, IgG3, IgG4, IgAQ1 and IgA2) or subclass of immunoglobulin molecule.

In some embodiments, antibody A and antibody B can be the same or different.

In some embodiments, antibody A or antibody B is selected from any one of cytotoxic antibodies, inhibitors of cell proliferation, regulators of cell activation and interaction, regulators of the human immune system, and neutralizations of antigens.

In some embodiments, antibody A or antibody B is selected from any one of a group consisting of anti-HER2 antibody, anti-CCR8 antibody, anti-FAP antibody, anti-OX-40 antibody, anti-41BB antibody, anti-Angiopoietin-2 antibody, anti-ant-IL-4Rα antibody, anti-BCMA antibody, anti-Blys antibody, anti-BTNO2 antibody, anti-C5 antibody, anti-CD122 antibody, anti-CD13 antibody, anti-CD133 antibody, anti-CD137 antibody, anti-CD138 antibody, anti-CD16a antibody, anti-CD19 antibody, anti-CD20 antibody, anti-CD22 antibody, anti-CD27 antibody, anti-CD28 antibody, anti-CD3 antibody, anti-CD30 antibody, anti-CD33 antibody, anti-CD38 antibody, anti-CD40 antibody, anti-CD47 antibody, anti-CD-8 antibody, anti-CEA antibody, anti-CGPR/CGRPR antibody, anti-CSPGs antibody, anti-CTLA4 antibody, anti-CTLA-4 domains antibody, anti-DLL-4 antibody, anti-EGFR antibody, anti-EpCAM antibody, anti-factor IXa antibody, anti-factor×antibody, anti-GITR antibody, anti-GP130 antibody, anti-Her3 antibody, anti-HSG antibody, anti-ICOS antibody, anti-IGF1 antibody, anti-IGF1/2 antibody, anti-IGF-1R antibody, anti-IGF2 antibody, anti-IGFR antibody, anti-IL-1 antibody, anti-IL-12 antibody, anti-IL-12p40 antibody, anti-IL-13 antibody, anti-IL-17A antibody, anti-IL-1β antibody, anti-IL-23 antibody, anti-IL-5 antibody, anti-IL-6 antibody, anti-IL-6R antibody, anti-Lag-3 antibody, anti-LAG3 antibody, anti-MAG antibody, anti-Met antibody, anti-NgR antibody, anti-NogoA antibody, anti-OMGp antibody, anti-OX40 antibody, anti-PD-1 antibody, anti-PDGFR antibody, anti-PDL-1 antibody, anti-PSMA antibody, anti-RGMA antibody, anti-RGMB antibody, anti-SARS-CoV-2 antibody, anti-Te38 antibody, anti-TIM-3 antibody, anti-TNF antibody, anti-TNFα antibody, anti-TROP-2 antibody, anti-TWEAK antibody, anti-VEGF antibody, and anti-VEGFR antibody.

In some embodiments, antibody A is an anti-FAP antibody, and antibody B is an anti-HER2 antibody. In some embodiments, antibody A is an anti-HER2 antibody trastuzumab, which is approved to treat breast cancer with human epidermal growth factor Receptor 2-positive (HER2+). The commercial name of this antibody is Herceptin. The sequence of the VH domain and VL of the anti-HER2 trastuzumab is shown in SEQ ID No.: 31 and SEQ ID No: 5, respectively. Antibody B is an anti-FAP antibody, it targets the fibroblast activation protein.

In some embodiments, half-body B is unchanged. In some embodiments, half-body B is changed.

In some embodiments, a tag is fused to half-body B; preferably, the tag is fused to the C terminus of light chain 2 of half-body B.

The tag is used to purify the target protein or label the target protein. The tag includes SUMO tag, HIS tag, Flag tag, HA tag, MYC tag, SBP tag, CBD tag, GST tag, MBP tag, pMAL tag, IMPACT tag, Protein A and GFP.

In some embodiments, a SUMO tag is fused to C-terminal of the light chain 2 in half-body B, separated by the thrombin cleavage sequence.

In some embodiments, half-body A is assembled to half-body B in any form.

In some embodiments, half-body A is assembled to half-body B in the form of knobs-into-holes.

In some embodiments, half-body A is assembled to half-body B in the form of knobs-into-holes combined with cysteine mutation.

“Knobs-into-holes” as used herein refers to the CH3 domain of one heavy chain with the knob mutations and the CH3 domain of the other heavy chain with the hole mutations. Generally, “Knobs-into-holes” refers to an intra-interface modification between two antibody heavy chains in the CH3 domains: i) in the CH3 domain of one heavy chain, residues substituted with amino acid bearing a large side chain, thereby creating a protrusion (“knob”) in the interface in the CH3 domain of the first heavy chain. ii) in the CH3 domain of the other heavy chain, amino acid residues are replaced with amino acid residues having a smaller side chain volume, thereby creating a cavity (“hole”) within the interface in the second CH3 domain, in which a protrusion (“knob”) in the first CH3 domain can be placed.

In some embodiments, half-body A is assembled to half-body B by knobs-into-holes in CH3 domains, wherein heavy chain 1 has the “knob” CH3 domain and is assembled to heavy chain 2, the CH3 domain of which has “hole” mutations. In some embodiments, the half-body A is assembled to half-body B by knobs-into-holes in CH3 domains, wherein heavy chain 1 has the “hole” CH3 domain and is assembled to heavy chain 2, the CH3 domain of which has “knob” mutations.

In some embodiments, provided herein is an isolated polynucleotide encoding the heavy chain 1, the light chain 1, the heavy chain 2 or the light chain 2 of the new protein according to the present disclosure.

In some embodiments, provided herein is a set of isolated polynucleotides, comprising the polynucleotide encoding the heavy chain 1, the polynucleotide encoding light chain 1, the polynucleotide encoding heavy chain 2 and the polynucleotide encoding light chain 2 of the new protein according to the present disclosure.

In some embodiments, provided herein is an isolated vector comprising the isolated polynucleotide according to the present disclosure.

In some embodiments, provided herein is a host cell comprising the isolated polynucleotide according to the present disclosure or the set of insolated polynucleotides the present disclosure or the isolated vector according to the present disclosure.

In some embodiments, provided herein is a pharmaceutical composition comprising the new protein according to the present disclosure or the isolated polynucleotide according to the present disclosure or the set of isolated polynucleotides according to the present disclosure or the isolated vector according to the present disclosure or the host cell according to the present disclosure, and a pharmaceutically acceptable carrier.

In some embodiments, provided herein is the use of the new protein according to the present disclosure or the isolated polynucleotide according to the present disclosure or the set of isolated polynucleotides according to the present disclosure or the isolated vector according to the present disclosure or the host cell according to the present disclosure or the pharmaceutical composition according to the present disclosure in the manufacture of a drug for preventing or treating a disease, or in the manufacture of a kit for diagnosing a disease.

In some embodiments, provided herein is a method of preventing or treating a disease in a subject in need thereof, comprising administrating to the subject a therapeutically effective amount of the new protein according to the present disclosure or the isolated polynucleotide according to the present disclosure or the set of isolated polynucleotides according to the present disclosure or the isolated vector according to the present disclosure or the host cell according to the present disclosure or the pharmaceutical composition according to the present disclosure.

In the present invention, provided herein is a method for production of the new protein according to the present disclosure, comprising introducing an expression vector of the heavy chain 1, an expression vector of the light chain 1, an expression vector of the heavy chain 2 and an expression vector of the light chain 2 together into an expression host cell, or a combination of the expression vectors into the separate expression host cells and expressing them under a proper condition.

In some embodiments, the method comprises introducing four expression vectors into the expression host and expressing four protein chains in one host under a proper condition, wherein the expression vector of heavy chain 1 contains the expression construction of heavy chain 1, the expression vector of light chain 1 contains the expression construction of light chain 1, the expression vector of heavy chain 2 contains the expression construction of heavy chain 2, the expression vector of light chain 2 contains the expression construction of light chain 2, wherein the expression construction can be a plasmid or other expression form.

In some embodiments, a correct pairing of the new protein is insensitive to the molar ratio of expression vectors of heavy chain 1:light chain 1:heavy chain 2:light chain 2. In some embodiments, the molar ratio of expression vector of heavy chain 1:light chain 1:heavy chain 2:light chain 2 has no limitation. The molar ratio of expression vectors of heavy chain 1:light chain 1:heavy chain 2:light chain 2 can be 1:1:1:1 or 2:2:1:1 or 1:1:2:2 or 1:3:1:1 or 1:1:1:3.

In some embodiments, the host cells are eukaryotic cells. In some embodiments, the host cells are mammalian cells.

In some embodiments, at least one pair of the homodimerization domains is selected from the combination of IgG1 CH3/CH3, IgM CH2/CH2 or IgE CH2/CH2.

In some embodiments, at least one pair of the heterodimerization domains are selected from a pair of HLA and β2m, a pair of β2m and CD1b, a pair of CD1a and β2m, a pair of GABA1 and GABA2, or a pair of SIRPα and CD47.

In some embodiments, the heterodimerization domains are selected from at least one of the combinations of HLA/β2m, β2m/HLA, CD1b/β2m, β2m/CD1b, β2m/CD1a and CD1a/β2m.

In some embodiments, the heterodimerization domains are selected from β2m/CD1b or CD1b/β2m or β2m/CD1a or CD1a/β2m, preferably the CH1 domain of heavy chain 1 is replaced with α3 domain of CD1a and CL of light chain 1 is replaced with β2m.

In some embodiments, β2m/CD1a or CD1a/β2m, or β2m/CD1b or CD1b/β2m comprises mutations. In some embodiments, β2m/CD1a or CD1a/β2m, or β2m/CD1b or CD1b/β2m comprise one or more disulfide bonds by introducing cysteine mutations.

In some embodiments, the CH1 domain of heavy chain 1 is replaced with CD1a^{D240C} and CL of light chain 1 is replaced with β2 m^{R12C}. In some embodiments, the CH1 domain of heavy chain 1 is replaced with CD1a^{S238C} and CL of light chain 1 is replaced with β2m^{R12C}. In some embodiments, the CH1 domain of heavy chain 1 is replaced with CD1a^{D234C} and CL of light chain 1 is replaced with β2m^{Q8C}. In some embodiments, the CH1 domain of heavy chain 1 is replaced with CD1a^{G194C} and CL of light chain 1 is replaced with β2m^{M99C}. In some embodiments, the CH1 domain of heavy chain 1 is replaced with CD1a^{W190C} and CL of light chain 1 is replaced with β2m^{P14C}. In some embodiments, the CH1 domain of heavy chain 1 is replaced with CD1a^{G194C} and CL of light chain 1 is replaced with β2m^{100C}. In some embodiments, the CH1 domain of the heavy chain 1 is replaced with CD1a^{G194C} and the CL of the light chain 1 is replaced with β2m^{Q8K/100C}. In some embodiments, the CH1 domain of the heavy chain 1 is replaced with CD1a^{G194C-G233S} and the CL of the light chain 1 is replaced with β2m^{Q8K/100C}. In some embodiments, the CH1 domain of the heavy chain 1 is replaced with CD1a^{G194C/G233S/D234E} and the CL of the light chain 1 is replaced with β2m^{Q8K/100C}. In some embodiments, the CH1 domain of the heavy chain 1 is replaced with CD1b^{G194C} and the CL of the light chain 1 is replaced with β2m^{Q8K/100C}. In some embodiments, the CH1 domain of the heavy chain 1 is replaced with CD1b^{G194C/G233S} and the CL of the light chain 1 is replaced with β2m^{Q8K/100C}. In some embodiments, the CH1 domain of the heavy chain 1 is replaced with CD1b^{G194C/G233S/D234E} and the CL of the light chain 1 is replaced with β2m^{Q8K/100C}.

In some embodiments, the new protein is selected from the protein ABC060, ABC061, ABC570, ABC571, ABC572, ABC132, ABC133, ABC074, ABC075, ABC131, ABC171, ABC172, ABC173, ABC174, ABC215, ABC588, ABC591, ABC270, ABC271, ABC272, ABC373, ABC374, ABC405, ABC478, ABC603, ABC672, ABC673, ABC513, ABC604, ABC605, ABC674ABC675, ABC736, ABC737, ABC738, ABC739, ABC723, ABC724, ABC752, or ABC753,
wherein ABC060 consists of four chains shown in SEQ ID Nos: 13, 11, 2 and 4, respectively, ABC061 consists of four chains shown in SEQ ID Nos: 14, 12, 2 and 4 respectively, ABC570 consists of four chains shown in SEQ ID Nos: 82, 83, 3 and 4 respectively, ABC571 consists of four chains shown in SEQ ID Nos: 84, 83, 3 and 4 respectively, ABC572 consists of four chains shown in SEQ ID Nos: 82, 85, 3 and 4 respectively, ABC132 consists of four chains shown in SEQ ID Nos: 14, 26, 2 and 4 respectively, ABC133 consists of four chains shown in SEQ ID Nos: 27, 11, 2 and 4 respectively, ABC074 consists of four chains shown in SEQ ID Nos: 22, 21, 2 and 4 respectively, ABC075 consists of four chains shown in SEQ ID Nos: 24, 23, 2 and 4 respectively, ABC131 consists of four chains shown in SEQ ID Nos: 19, 20, 2 and 4 respectively, ABC171 consists of four chains shown in SEQ ID Nos: 14, 25, 3 and 1 respectively, ABC172 consists of four chains shown in SEQ ID Nos: 14, 26, 3 and 1 respectively, ABC173 consists of four chains shown in SEQ ID Nos: 27, 11, 3 and 1 respectively, ABC174 consists of four chains shown in SEQ ID Nos: 28, 11, 3 and 1 respectively, ABC734 consists of four chains shown in SEQ ID Nos: 13, 11, 3 and 1 respectively, ABC735 consists of four chains shown in SEQ ID Nos: 14, 12, 3 and 1 respectively, ABC215 consists of four chains shown in SEQ ID Nos: 38, 41, 45 and 44 respectively, ABC588 consists of four chains shown in SEQ ID Nos: 27, 11, 47 and 46 respectively, ABC591 consists of four chains shown in SEQ ID Nos: 36, 37, 47 and 46 respectively, ABC270 consists of four chains shown in SEQ ID Nos: 66, 65, 3 and 1 respectively, ABC271 consists of four chains shown in SEQ ID Nos: 67, 65, 3 and 1, ABC272 consists of four chains shown in SEQ ID Nos: 68, 69, 3 and 1 respectively, ABC373 consists of four chains shown in SEQ ID Nos: 73, 71, 3 and 1 respectively, ABC374 consists of four chains shown in SEQ ID Nos: 72, 70, 3 and 1 respectively, ABC405 consists of four chains shown in SEQ ID Nos: 73, 74, 3 and 1 respectively, ABC478 consists of four chains shown in SEQ ID Nos: 76, 74, 3 and 1 respectively, ABC603 consists of four chains shown in SEQ ID Nos: 76, 75, 3 and 1 respectively, ABC672 consists of four chains shown in SEQ ID Nos: 77, 75, 3 and 1 respectively, ABC673 consists of four chains shown in SEQ ID Nos: 78, 75, 3 and 1 respectively, ABC513 consists of four chains shown in SEQ ID Nos: 79, 74, 3 and 1 respectively, ABC604 consists of four chains shown in SEQ ID Nos: 80, 74, 3 and 1 respectively, ABC605 consists of four chains shown in SEQ ID Nos: 80, 75, 3 and 1 respectively, ABC674 consists of four chains shown in SEQ ID Nos: 81, 75, 3 and 1 respectively, ABC675 consists of four chains shown in SEQ ID Nos: 59, 75, 3 and 1 respectively, ABC736 consists of four chains shown in SEQ ID Nos: 86, 75, 2 and 1 respectively, ABC737 consists of four chains shown in SEQ ID Nos: 87, 75, 2 and 1 respectively, ABC738 consists of four chains shown in SEQ ID Nos: 88, 75, 2 and 1 respectively, ABC739 consists of four chains shown in SEQ ID Nos: 89, 75, 2 and 1 respectively, ABC723 consists of four chains shown in SEQ ID Nos: 76, 75, 47 and 46 respectively, ABC724 consists of four chains shown in SEQ ID Nos: 59, 75, 47 and 46 respectively, ABC752 consists of four chains shown in SEQ ID Nos: 106, 105, 47 and 46 respectively, and ABC753 consists of four chains shown in SEQ ID Nos: 107, 105, 47 and 46 respectively.

Preferably, the new protein is selected from the protein ABC570, ABC571, ABC572, ABC132, ABC133, ABC074, ABC075, ABC131, ABC171, ABC172, ABC173, ABC174, ABC215, ABC588, ABC591, ABC270, ABC271, ABC272, ABC373, ABC374, ABC405, ABC478, ABC603, ABC672, ABC673, ABC513, ABC604, ABC605, ABC674, ABC675, ABC736, ABC737, ABC738, ABC739, ABC723, ABC724, ABC752, or ABC753.

In some embodiments, part of half-body A and/or part of half-body B is replaced by at least one pair of the homo/heterodimerization domains, and each pair of the homo/heterodimerization domains can form a homo/heterodimer themselves alone or in conjunction with other proteins, preferably, the CH1/CL in the half-body A or the CH1/CL in half-body B is replaced by at least one pair of the homo/heterodimerization domains. In some embodiments, CH2 of the heavy chain 1 and CH2 of the heavy chain 2 are replaced by at least one pair of the homo/heterodimerization domains. In some embodiments, CH3 of the heavy chain 1 and CH3 of the heavy chain 2 are replaced by at least one pair of the homo/heterodimerization domains.

In some embodiments, the homo/heterodimerization domains may be introduced by other means than replacement. In some embodiments, the homo/heterodimerization domains may be introduced by an insertion.

The term “insertion”, as used herein, refers to adding one or more amino acid residues or domains between two existing amino acids.

In some embodiments, the insertion may be carried out by inserting homo/heterodimerization domains into an antibody, a fragment or a variant thereof. For example, homo/heterodimerization domains may be directly inserted between the CH1/CL domains and the CH2/CH2 domains. For one more example, homo/heterodimerization domains may be directly inserted between the CH1/CL domains and VH/VL domains. Another example would be homo/heterodimerization domain may be directly inserted following the CH3/CH3 domains. In some embodiments, at least one pair of the homo/heterodimerization domains are inserted in the new protein, and each pair of the homo/heterodimerization domains can form a homodimer or heterodimer themselves alone or in conjunction with other proteins.

In some embodiments, at least one pair of the homo/heterodimerization domains may be fused to the new protein, and each pair of the homo/heterodimerization domains can form a homodimer or heterodimer themselves alone or in conjunction with other proteins.

The term “fusion”, as used herein, may refer to connecting a protein with another biological material, such as a protein, a nucleic acid molecule, or any other biological molecule or part thereof. A fusion protein may be defined as a protein consisting of at least two joined domains.

In some embodiments, at least one pair of the homo/heterodimerization domains are fused to the new protein, and each pair of the homo/heterodimerization domains can form a homodimer or heterodimer themselves alone or in conjunction with other proteins. In some embodiments, at least one pair of the homo/heterodimerization domains are fused to any two chains of the new protein. In some embodiments, at least one pair of the homo/heterodimerization domains are fused to light chain 1 and/or heavy chain 1. In some embodiments, at least one pair of the homo/heterodimerization domains are fused to heavy chain 1 and/or heavy chain 2. In some embodiments, at least one pair of the homo/heterodimerization domains are fused to light chain 2 and/or heavy chain 2. In some embodiments, two pairs of the homo/heterodimerization domains are fused to light chain 1 and heavy chain 1 in half-body A, and light chain 2 and heavy chain 2 in half-body B, respectively.

In some embodiments, the CH1/CL in half-body A and/or the CH1/CL in half-body B is replaced by at least one pair of the homo/heterodimerization domains, and each pair of the homo/heterodimerization domains can form a homodimer or heterodimer themselves alone or in conjunction with other proteins.

In some embodiments, a protein may be mutated by fusing another homo/heterodimerization domain. For example, an IgG antibody may be fused with another homo/heterodimerization domain. For one more example, an IgG antibody may be fused with another homo/heterodimerization domains at the N-terminal of the IgG antibody. In some embodiments, an IgG antibody may be fused with another homo/heterodimerization domain at the C-terminal of the IgG antibody, or within a domain thereof. For another example, the homo/heterodimerization domains may be fused at the VH/VL or CH1/CL domain of an antibody or a half-body. For another example, the homo/heterodimerization domains may be fused at the CH1/CL domain in a half-body by a covalent bond, such as a disulfide bond.

The term “thermal stability” as used herein relates to the ability of the protein, e.g., antibodies, to resist the action of heat and to maintain its properties, such as strength, toughness, or elasticity at given temperature. During the denaturation process caused by heating, the whole protein or domains of proteins undergo one or several unfolding event which gives rise one or several transitions in melting curves.

In some embodiments, the “Tm” may be any one, any two or all of Tm1, Tm2, Tm3.

The term “Tm1”, as used herein, refers to the first melting/unfolding transition midpoint.

The term “Tm2”, as used herein, refers to the second melting/unfolding transition midpoint.

The term “Tm3”, as used herein, refers to the third melting/unfolding transition midpoint.

In some embodiments, provided herein is a method for production of a new protein with an adjusted thermal stability, especially with a substantially the same or improved thermal stability, especially with a substantially the same or improved Tm compared with an original protein, wherein the original protein has four chains of polypeptides, the four chains of polypeptides are heavy chain 1, light chain 1, heavy chain 2 and light chain 2 respectively, wherein the heavy chain 1 and the heavy chain 2 comprise VH domain, CHI domain, CH2 domain and CH3 domain and form a long chain of VH-CH1-CH2-CH3 respectively; the light chain 1 and the light chain 2 comprise VL domain and CL domain and form a short chain of VL-CL respectively; wherein heavy chain 1 and light chain 1 are derived from an antibody A, the single heavy chain 1 pairs with the single light chain 1 and forms half-body A; heavy chain 2 and a light chain 2 are derived from an antibody B, the single heavy chain 2 pairs with the single light chain 2 and forms half-body B; wherein CHI domain of heavy chain 1 pairs with CL domain of light chain 1, named herein as a combination of CH1/CL of half-body A, CH1 domain of heavy chain 2 pairs with CL domain of light chain 2, named herein as a combination of CH1/CL of half-body B; and the method is characterized in that the combination CH1/CL in half-body A is replaced with homodimerization domains or other heterodimerization domains.

In some embodiments, the half-body B is unchanged. In some embodiments, the half-body B is changed.

In some embodiments, the Tm is substantially the same or improved by at least 0.1° C., 0.2° C., 0.3° C., 0.4° C., 0.5° C., 0.6° C., 0.7° C., 0.8° C., 0.9° C., 1° C., 1.5° C., 2° C., 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., preferably at least 0.3° C., 0.5° C., 1.5° C., 2° C., or 3° C. compared with the original protein. In some embodiments, the Tm is increased or decreased by at least 0.3° C., 0.5° C., 1.5° C., or 3° C. compared with the original protein. In some embodiments, the Tm is increased or decreased by more than 10° C. compared with the original protein.

In some embodiments, the thermal stability of a mutant antibody is improved when the mutant antibody keeps substantially the same Tm value compared to the original antibody. In some embodiments, the thermal stability of a mutant antibody is improved when the mutant antibody increases the Tm value compared to the original antibody.

In some embodiments, provided herein is a method to produce a new protein with improved thermal stability, wherein the heterodimerization domains can be derived from human or other mammals, and the heterodimerization domains are not TCR α/β or TCR β/α constant domains. In some embodiments, provided herein is a method to produce a new protein with improved thermal stability, wherein the heterodimerization domains can be derived from human or other mammals, and the heterodimerization domains are not TCR α/β or TCR β/α constant domains or HLA/β2m or β2m/HLA domains.

In some embodiments, provided herein is a method to produce a new protein with improved thermal stability, wherein the heterodimerization domains are selected from a pair of HLA and β2m, a pair of β2m and CD1b, a pair of CD1a and β2m, a pair of GABA1 and GABA2, or a pair of SIRPα and CD47.

In some embodiments, provided herein is a method to produce a new protein with improved thermal stability, wherein the heterodimerization domains are selected from at least one of the combinations of HLA/β2m, β2m/HLA, CD1b/β2m, β2m/CD1b, β2m/CD1a and CD1a/β2m.

In some embodiments, provided herein is a method to produce a new protein with improved thermal stability, wherein the heterodimerization domains are selected from β2m/CD1b or CD1b/β2m or β2m/CD1a or CD1a/β2m, preferably the CH1 domain of heavy chain 1 is replaced with α3 domain of CD1a and CL of light chain 1 is replaced with β2m.

In some embodiments, provided herein is a method to produce a new protein with improved thermal stability, wherein the homodimerization domains can be derived from human or other mammals, and the homodimerization domains are selected from IgG CH3/CH3 domain, IgM CH2/CH2 domain or IgE CH2/CH2 domain.

In some embodiments, provided herein is a method to produce a new protein with improved thermal stability, further changing the new protein's amino acid by mutagenesis.

In some embodiments, provided herein is a method to produce a new protein with improved thermal stability, wherein the amino acid is changed by introducing an H-bond or a disulfide bond to the antibody, preferably, to the CH1/CL of half-body A.

In some embodiments, provided herein is a method to produce a new protein with improved thermal stability, wherein β2m/CD1a or CD1a/β2m, or β2m/CD1b or CD1b/β2m comprises mutations.

In some embodiments, provided herein is a method to produce a new protein with improved thermal stability, wherein β2m/CD1a or CD1a/β2m, or β2m/CD1b or CD1b/β2m bears mutations that can form one or more disulfide bonds as well as better domain packing.

In some embodiments, provided herein is a method to produce a new protein with improved thermal stability, wherein the CH1 domain of heavy chain 1 is replaced with CD1a^{D240C} and CL of light chain 1 is replaced with β2 m^{R12C}. In some embodiments, provided herein is a method for production of a new protein with improved thermal stability, wherein the CH1 domain of heavy chain 1 is replaced with CD1a^{S238C} and CL of light chain 1 is replaced with β2 m^{R12C}. In some embodiments, provided herein is a method for production of a new protein with improved thermal stability, wherein the CH1 domain of heavy chain 1 is replaced with CD1a^{D234C} and CL of light chain 1 is replaced with β2m^{Q8C}. In some embodiments, provided herein is a method for production of a new protein with improved thermal stability, wherein the CH1 domain of heavy chain 1 is replaced with CD1a^{G194C} and CL of light chain 1 is replaced with β2m^{M99C}. In some embodiments, provided herein is a method for production of a new protein with improved thermal stability, wherein the CH1 domain of heavy chain 1 is replaced with CD1a^{W190C} and CL of light chain 1 is replaced with β2m^{P14C}. In some embodiments, provided herein is a method for production of a new protein with improved thermal stability, wherein the CH1 domain of heavy chain 1 is replaced with CD1a^{G194C} and CL of light chain 1 is replaced with β2m^{100C}.

In some embodiments, provided herein is a method for production of a new protein with improved thermal stability and/or yield, wherein the CH1 domain of heavy chain 1 is replaced with CD1a^{G194C} and CL of light chain 1 is replaced with β2m^{100C/Q8K}, or the CH1 domain of heavy chain 1 is replaced with CD1a^{G194C/G233S} and CL of light chain 1 is replaced with β2m^{100C/Q8K}, or the CH1 domain of heavy chain 1 is replaced with CD1a^{G194C/G233S/D234E} and CL of light chain 1 is replaced with β2m^{100C/Q8K}.

In some embodiments, provided herein is a method for production of a new protein with improved thermal stability and/or yield, wherein the CH1 domain of heavy chain 1 is replaced with CD1b^{G194C} and CL of light chain 1 is replaced with β2m^{100C}. In some embodiments, provided herein is a method for production of a new protein with improved thermal stability and/or yield, wherein the CH1 domain of heavy chain 1 is replaced with CD1b^{G194C} and CL of light chain 1 is replaced with β2m^{100C/Q8K} or the CH1 domain of heavy chain 1 is replaced with CD1b^{G194C/G233S} and CL of light chain 1 is replaced with β2 m^{100C/Q8K}, or the CH1 domain of heavy chain 1 is replaced with CD1b^{G194C/G233S/D234E} and CL of light chain 1 is replaced with β2 m^{100C/Q8K}.

The thermal stability can be measured by any technique apparent to those of skill in the art. In some embodiments, the new protein with improved thermal stability has a melting temperature within about 5° C. of the corresponding parent protein, as described herein. In some embodiments, the new protein with improved thermal stability has a melting temperature within about 4° C. of the corresponding parent protein, as described herein. In some embodiments, the new protein with improved thermal stability has a melting temperature within about 3° C. of the corresponding parent protein, as described herein. In some embodiments, the new protein with improved thermal stability has a melting temperature within about 2° C. of the corresponding parent protein, as described herein. In some embodiments, the new protein with improved thermal stability has a melting temperature within about 1° C. of the corresponding parent protein, as described herein.

In some embodiments, the new protein with improved thermal stability has a melting temperature at least about 5° C. greater than the corresponding parent protein, as described herein. In some embodiments, the new protein with improved thermal stability has a melting temperature at least about 4° C. greater than the corresponding parent protein, as described herein. In some embodiments, the new protein with improved thermal stability has a melting temperature at least about 3° C. greater than the corresponding parent protein, as described herein. In some embodiments, the new protein with improved thermal stability has a melting temperature at least about 2° C. greater than the corresponding parent protein, as described herein. In some embodiments, the new protein with improved thermal stability has a melting temperature at least 1° C. greater than the corresponding parent protein, as described herein.

In some embodiments, the new protein is produced with a yield which is at least 1.1, 1.2, 1.3, 1.4, 1.5, 1.6, 1.7, 1.8, 1.9, 2, 3, 4, 5, 6, 7, 8, 9, 10, 20, 30, 40, 50, 60, 70, 80, 90, 100 times of yield of the corresponding parent protein.

### BRIEF DESCRIPTION OF THE DRAWINGS

The following is a brief description of the drawings, which are presented for the purposes of illustrating the exemplary embodiments disclosed herein and not for the purposes of limiting the scope of the claims.

FIG. 1 shows the screening of the homo/heterodimerization domains or proteins for substituting the CH1/CL of half-body A.

FIG. 1A shows the non-reducing and reducing SDS-PAGE gel analysis of protein A-purified anti-HER2×FAP hetero-IgG variants, in which the CH1/CL of half-body A is replaced with non-IgG-like heterodimer domains. The heterodimer domain replacements are Fos/Jun (ABC064), GABA1/GABA2 (ABC074) and GABA2/GABA1 (ABC076).

FIG. 1B shows the non-reducing and reducing SDS-PAGE gel analysis of protein A-purified anti-HER2×FAP hetero-IgG variants, in which the CH1/CL of half-body A is replaced with IgG-like domains. The heterodimer domain replacements are IL2Rβ/γ (ABC058, ABC059), and CD8a/CD8β (ABC134).

FIG. 1C shows the non-reducing and reducing SDS-PAGE gel analysis of protein A-purified anti-HER2×FAP hetero-IgG variants, in which the CH1/CL in half-body A is replaced with IgG-like homodimer domains. The homodimer domain replacements are CH3^{IgG}/CH3^{IgG} (ABC055), knob^{IgGCH3}/hole^{IgGCH3} (ABC056), knob^{IgGCH3}/hole^{IgGCH3} (ABC057), CH2^{IgM}/CH3^{IgM} (ABC570), hole^{IgMCH2}/knob^{IgMCH2} (ABC571), knob^{IgMCH2}/hole^{IgMCH2} (ABC572). Wherein knob^{IgGCH3}/hole^{IgGCH3} means the CH1 domain of heavy chain 1 (HCl) is replaced with IgG1 CH3 domain with knob mutation (S354C, T336W) and CL of light chain 1 (LC1) is replaced with IgG1 CH3 domain with hole mutations (Y349C, T336S, L368A, Y407V). Wherein knob^{IgMCH2}/hole^{IgMCH2} means the CH1 domain of heavy chain (HCl) is replaced with IgM CH2 domain with knob mutations (R245C, C337S, I258Y, T302Y, the residue numbering is based on Uniprot PODOX6) and CL of light chain 1 (LC1) is replaced with IgM CH2 domain with hole mutation. All anti-HER2×FAP hetero-IgG variants (IgG domains) were analyzed after protein A purification under non-reducing, reducing treated conditions on 4-20% SDS-PAGE gel.

FIG. 1D shows the non-reducing and reducing SDS-PAGE gel analysis of protein A-purified anti-HER2×FAP hetero-IgG variants, in which the IgG-like heterodimeric domains replaced the CH1/CL in half-body A. The heterodimer domains are HLA/β2m (ABC060), β2m/HLA (ABC061), SIPRα/CD47 (ABC131), β2m/CD1b (ABC132) and CD1a/β2m (ABC133). The deglycosylated anti-HER2×FAP hetero-IgG variants after PNGaseF treatment were also analyzed.

FIG. 2 shows the SDS-PAGE gel analysis of anti-HER2×FAP hetero-IgG with varied LC1/LC2 chain ratio. Four combinations (β2m/CD1a, CD1a/β2m, β2m/CD1b and CD1b/β2m) were tested. Four plasmids in different ratios (Table 3) were co-transfected into one host cell line. Protein A-purified products were analyzed under non-reducing and reducing conditions on 4-20% SDS-PAGE gel. The results indicate the orthogonal Fab pairing is insensitive to the LC1/LC2 ratio.

FIG. 3A shows the SDS-PAGE gel analysis of ABC171-174, ABC734 and ABC735 under non-reducing and reducing conditions. The chain ratio used in all designs was 1:1:1:1.

FIG. 3B shows the SEC-HPLC chromatogram of BsAbs and mAb after protein A purification. The percentage of desired product was labeled. Herceptin (ABC141), anti-FAP×HER2-CrossMab BsAb (ABC203) and anti-FAP×HER2-Wuxibody BsAb (ABC204), anti-FAP×HER2-HLA/β2m (ABC734), anti-FAP×HER2-HLA/β2m (ABC735) were used as control.

FIG. 3C shows the sequence alignment of the α3 domain (AA: 179-278) of common HLA-A and HLA-B allotypes.

FIG. 4 shows the thermal stability of anti-HER2/FAP BsAb variants and their parental monoclonal IgGs. FIG. 4A: The normalized melting curve of antibodies recorded using a biosystems real-time PCR system. FIG. 4B: The derivative (dF/dT) plot of the melting curve. The tip value of the peak represents the derivative Tm value. FIG. 4C summarizes the melting temperate (Tm) measured by differential scanning fluorimetry.

FIG. 5 shows the LC-MS analysis of intact ABC171-ABC174 bispecific antibodies on Quadrupole time-of-flight (Q-TOF) liquid mass spectrometer in non-reducing mode. Since LC1 is not covalently bound to the rest three chains, two peaks were detected in non-reducing mode. Mass spectrometric analysis of IgG BsAb showed correct chain assembly. FIG. 5A: The deconvoluted mass spectrum of ABC171. The theoretical molecular weight (MW) of LC1 is 23340.26 Da, and the observed mass of LC1 is 23340.00 Da. The theoretical MW of HC1/HC2/LC2 is 122622.17 Da, and the observed mass of HC1/HC2/LC2 is 122622.25 Da. FIG. 5B: The deconvoluted mass spectrum of ABC173. The theoretical molecular weight (MW) of LC1 is 23662.51 Da, and the observed mass of LC1 is 23662.75 Da. The theoretical MW of HC1/HC2/LC2 is 121985.58 Da, and the observed mass of HC1/HC2/LC2 is 121986.5 Da. FIG. 5C: The deconvoluted mass spectrum of ABC172. The theoretical molecular weight (MW) of LC1 is 24722.80 Da, and the observed mass of LC1 is 24721.4 Da. The theoretical MW of HC1/HC2/LC2 is 122622.17 Da, and the observed mass of HC1/HC2/LC2 is 122620.4 Da.

FIG. 6 shows Bio-Layer Interferometry (BLI)-based binding kinetics analysis of the anti-HER2×anti-FAP BsAbs to human HER2 antigen. Processed kinetic data sets are presented for ABC141 (Herceptin, the positive control (A), ABC171 (B), ABC172 (C), ABC173 (D) and ABC174 (E) binding to human antigen HER2. Smooth lines represent a global fit of the data to a 1:1 interaction model.

FIG. 7 shows BLI-based simultaneous binding of anti-HER2×FAP Abio BsAb to human HER2 and human FAP antigens. The cartoon on the top represents the sequence of analyte loading. Biotinylated human HER2 was first immobilized on the SA sensor tip, followed by loading BsAbs and then dipping into the solution of the second antigen human FAP. ABC171, ABC172 and ABC173 were shown as examples. ABC141 (Herceptin), ABC089 (an isotype control antibody) and assay buffer was used as controls.

FIG. 8 shows the engineered Fab in ABC173 format displays similar conformation to the WT Fab. FIG. 8A: The cartoon structure of the Herceptin Fab domain (PDB ID: 6MH2). FIG. 8B: The predicted structure of engineered Fab in ABC173 by AlphaFold2. FIG. 8C: Alignment of the WT Fab and engineered Fab in ABC173 format.

FIG. 9 shows the engineered orthogonal Fab designs are broadly applicable across different antibody pairs.

FIG. 9A: the SDS-PAGE gel analysis of anti-Ab1/Ab2 antibodies in the ABC173, CrossMab and Wuxibody formats under non-reducing and reducing conditions. The Ab1/Ab2 combination mCD20/mCD3 means the variable domain sequence of half-body A is from the antibody against mouse CD20 and the variable domain of half-body B is from the antibody against mouse CD3, wherein CH1/CL of half-body A is replaced with CD1a/β2m (ABC215, labeled as 173), or CL/CH1 (ABC216, labeled as CrossMab), or TCR β/α constant domain (ABC217, labeled as Wuxi). In the HER2/hCD3 combination, the variable domain of half-body A is from Herceptin and the variable domain of half-body Bis from the anti-CD3 moiety in IMCgp100-CD3, wherein CH1/CL of half-body A is replaced with CD1a/β2m (ABC588, labeled as 173), or CL/CH1 (ABC589, labeled as CrossMab), or TCR β/α constant domain (ABC590, labeled as Wuxi). In the hCCR8/hCD3 combination, the variable domain of half-body A is from anti-hCCR8-antibody ABC138 (the light chain of the anti-hCCR8-antibody is shown in SEQ ID NO: 37, and the heavy chain of the anti-hCCR8 antibody is shown in SEQ ID NO: 36), and the variable domain of half-body B is from anti-CD3 moiety in IMCgp100-CD3, wherein CH1/CL of half-body A is replaced with CD1a/β2m (ABC591, labeled as 173), or CL/CH1 (ABC592, labeled as CrossMab) or TCR β/α constant domain (ABC593, labeled as Wuxi).

FIG. 9B: SEC-HPLC analysis of ABC215, ABC588 and ABC591 after protein A purification.

FIG. 9C: The deconvoluted mass spectrum of ABC588. The theoretical molecular weight (MW) of LC1 is 23662.1 Da, the observed mass of LC1 is 23662.5 Da. The theoretical MW of HC1/HC2/LC2 is 123134.8 Da, the observed mass of HC1/HC2/LC2 is 123133.4 Da.

FIG. 10 shows increased thermal stability by introducing a disulfide bond between CD1a α3 domain and β2m. FIG. 10A: illustrate the interactions between CD1a α3 domain and β2m (PDB ID: 1XZO). Cysteine mutations are introduced to enhance the interactions between CD1a α3 domain and β2m based on the structure (\textcircled{1}\textcircled{2}\textcircled{3}\textcircled{4}). FIG. 10B: Six pairs of cysteine mutations that are CD1a^{D240C}/β2m^{R12C} (ABC270), CD1a^{S238C}/β2m^{R12C} (ABC271), CD1a^{D234C}/β2m^{Q8C} (ABC272), CD1a^{G194C}/β2m^{M99C} (ABC373), CD1a^{W190C}/β2m^{P14C} (ABC374) and CD1a^{G194C}/β2 m^{100C} (ABC405) resulted ~150 kDa product revealed by the non-reducing SDS-PAGE. The position numbering is based on the crystal structure of CD1a/β2m (PDB ID 1XZO).

FIG. 10C: ABC405 demonstrated the most promising among the six tested variants regarding expression yield and thermal stability.

FIG. 11: Optimization of the bispecific variants for purity and stability. FIG. 11A shows the non-reducing and reducing SDS-PAGE gel of bispecific antibodies designed based on ABC173 format. FIG. 11B shows the non-reducing and reducing SDS-PAGE gel of bispecific antibodies designed based on ABC174 format. FIGS. 11C and 11D show the SEC-HPLC analysis of these variants after protein-Apurification. FIGS. 11E and 11F show the deconvoluted mass spectrum of mutant ABC603 and ABC605. The theoretical molecular weight (MW) of ABC603 is 145274.8 Da, and the observed mass of ABC603 is 145274.5 Da. The theoretical MW of ABC605 is 145226.1 Da, and the observed mass of ABC603 is 145225.0 Da.

FIG. 12: Thermal stability analysis of bispecific variants FIGS. 12A and 12B: The raw melting curve (FIG. 12A) of bispecific antibodies based on ABC173 format recorded by the biosystems real-time PCR systems and the derivative (dF/dT) plot of the melting curve (FIG. 12B) processed by the biosystems real-time PCR systems. FIGS. 12C and 12D: The raw melting curve (FIG. 12C) of bispecific antibodies based on ABC174 format recorded by the biosystems real-time PCR systems and the derivative (dF/dT) plot of the melting curve (FIG. 12D) processed by the biosystems real-time PCR systems. FIGS. 12E and 12F: The raw melting curve (FIG. 12E) of bispecific antibodies without N297A recorded by the biosystems real-time PCR systems and the derivative (dF/dT) plot of the melting curve (FIG. 12E) processed by the biosystems real-time PCR systems. ABC001 (anti-FAP parental antibody) and ABC141 (Herceptin, anti-HER2 parental antibody) was included for comparison.

FIGS. 12G and 12H: The raw melting curve (FIG. 12G) of Herceptin Fab variants (the CH/CL was replaced by CD1a/β2m) and Fab WT recorded by the biosystems real-time PCR systems and the derivative (dF/dT) plot of the melting curve (FIG. 12H) processed by the biosystems real-time PCR systems.

FIG. 13 Cell-based binding affinity assays to show the dual-binding of bispecific antibodies in the format of ABC603, ABC605, ABC673 or ABC675. FIG. 13A: The binding curves of ABC723_HER2/hCD3_603, ABC724_HER2/hCD3_605 and the parental antibody ABC141_Herceptin to HER2 antigen (SKBR3 cells). FIG. 13B: The binding curves of ABC723_HER2/hCD3_603, ABC724_HER2/hCD3_605 and the parental antibody ABC728_anti-hCD3 to hCD3 antigen (Jurkat cells). FIG. 13C: The binding curves of ABC752_hCCR8/hCD3_673, ABC753_hCCR8/hCD3_675 and the parental antibody ABC138_anti-hCCR8 to hCCR8 antigen (hCCR8-transfected 293T cells). FIG. 13D: The binding curves of ABC752_hCCR8/hCD3_673, ABC753_hCCR8/hCD3_675 and the parental antibody ABC728_anti-hCD3 to hCD3 antigen (Jurkat cells).

### DETAILED DESCRIPTION

The present disclosure is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following description is intended to illustrate some particular embodiments of the invention, and not to exhaustively specify all permutations, combinations and variations thereof.

Unless defined otherwise, all technical and scientific terms used herein have the same meaning as commonly understood by one of ordinary skill in the art to which the disclosure pertains. Although any methods and materials similar or equivalent to those described herein may be used in practice for testing of the present disclosure, the preferred materials and methods are described herein. In describing and claiming the present disclosure, the following terminology will be used.

As used herein, the term “antibody” is thus used to refer to any antibody-like molecule that has an antigen binding region, and this term includes antibody fragments that comprise an antigen binding domain such as Fab, F (ab′) 2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific); sc-diabody; kappa (lambda) bodies (scFv-CL fusions); DVD-Ig (dual variable domain antibody, bispecific format); SIP (small immunoprotein, a kind of mini-body); SMIP (small modular immune-pharmaceutical); scFv-Fc dimer; DART (ds-stabilized diabody “Dual Affinity ReTargeting”); all antibody mimetics comprising one or more CDRs and the like. The techniques for preparing and using various antibody-based constructs and fragments are well known in the art (see Kabat et al., 1991, specifically incorporated herein by reference).

Here as an example, we utilized a Fab fragment that binds to FAP (Fibroblast activation protein), with knob mutations on CH3 domain and a Fab fragment that binds to HER2 (Human Epidermal growth factor Receptor 2) with hole mutations on CH3 domain and N297A on CH2. Knobs-into-holes or other CH3/CH3 mutations that contribute the dimerization were introduced into the heavy chains (HCs) for heterodimeric Fc assembly.

As used herein, the term “bispecific antibody” means an antibody which comprises specificity for two target molecules, i.e. an antibody having specificities for at least two different epitopes or two different targets, typically non-overlapping epitopes.

“multi-specific antibody” means an antibody which comprises specificity for more than one target molecules, i.e., an antibody having specificities for at least two, or three or much more different epitopes or targets.

“IgG type antibody” means an antibody belongs to IgG immunoglobulin that comprises two light chains and two heavy chains. The light chain comprises VL and CL domains. The heavy chain comprises VH, CH1, CH2 and CH3 domains. Also, it can be defined as a four chains protein comprising the first chain, the second chain, the third chain and the fourth chain. The first and the fourth chains are VL-CL chains, the second and the third chains are VH-CH1-CH2-CH3 chains.

“Human homo/heterodimerization domain” means domains from human protein that can form a homo/heterodimer either as an intact protein or part of a full-length protein.

“Homo/heterodimerization domain” means homodimerization domain and/or heterodimerization domain.

“Homo/heterodimer” means homodimer and/or heterodimer.

“Knobs-into-holes” as used herein refers to the strategy for engineering antibody heavy chain homodimers for heterodimerization and also refers to the domain of HC1 and HC2 bearing the “knob” and “hole” amino acid mutations, respectively, or the other way around.

“Trastuzumab” as used herein refers to the approved monoclonal antibody (Herceptin) against human HER2 protein.

“SUMO tag” as used herein refers to sumo protein that fused to the C-terminus of LC2.

“Beta-2 microglobulin (β2m)” as used herein refers to the full length of human β2m (aa: 1-99) protein.

“The extracellular a3 domain of MHC class I” as used herein refers to the α3 domain (aa: 180-278) of human MHC class I proteins.

“Wuxibody” as used herein refers to the bispecific antibody format described in WO2019057122 A1, wherein CH1/CL of half-body A is replaced with TCR β/α constant domain.

“Anti-HER2×anti-FAP BsAb” means the bispecific antibody can bind to antigen HER2 and antigen FAP, wherein the VH/VL of half-body A is against antigen HER2 and VH/VL of half-body B is against antigen FAP.

“Half-body” refers to the dimer in which the light chain associates with its cognate heavy chain.

“CrossMab” is the bispecific antibody format invented by Roche (US20170129962 A1), wherein the CH1/CL of half-body A is replaced by each other.

“Abio antibody”, “Abio platform” and “Abio antibody platform” are used interchangeably herein to refer to any antibody (including but not limited to bispecific antibody and multi-specific antibody) in any format (whether IgG type antibody or not) in which CH1 and CL of a half-body are replaced with CD1s and β2m or vice versa. CD1s includes but not limited to CD1a and CD1b. CD1s and β2m can be wild-type or mutant.

“BsAbs” as used herein refers to bispecific immunoglobulin G format, which contains one binding moiety for each antigen. Exemplary BsAb formats include but are not limited to CrossMab, DAF (two-in-one), DAF (four-in-one), DutaMab, DT-IgG, knobs-in-holes common LC, knobs-in-holes assembly, charge pair, DuoBody, SEEDbody, triomab, LUZ-Y, Fcab, K-body, orthogonal Fab. Approved BsAbs include anti-EGFR/anti-cMET Amivantamab (GenMab, Janssen Biotech), anti-BCMA/anti-CD3 Teclistamab (GenMab and Jansen Biotech), anti-FactorX/anti-Factor IX Emicizumab (Roche), anti-VEGFA/anti-ANG2 Fabricimab (Roche), and anti-CD20/anti-CD3 Mosunetuzumab (Roche). In some embodiments, BsAb comprises heavy chains that are engineered for heterodimerization. For example, heavy chains can be engineered for heterodimerization using knobs-into-holes (KIH) (Ridgway et al., 1996), electrostatic steering of CH3 (Gunasekaran et al., 2010), the SEED technology (Davis et al., 2010), the XmAb bispecific platform (Moore et al., 2019), the Azymetric platform (Escobar-Cabrera et al., 2017), the DuoBody (Labrijn et al., 2013) and other heterodimeric Fe technologies.

“Abio BsAb”, “Abio BsIgG”, “Abio bispecific antibody”, “Abio bispecifics”, “Abio BsAb format” and “Abio bispecific format” are used interchangeably herein to refer to a bispecific immunoglobulin G in which CH1 and CL of a half-body are replaced with CD1s and β2m or vice versa. CD1s and β2m can be wild-type or mutant. In one embodiment, CD1s represents CD1a or CD1b. In some embodiments, the C-terminus of a light chain of Abio BsAb is fused with a tag, such as a SUMO tag or an Avi-tag.

“ABC173 format” or “173 format” used herein refers to a bispecific immunoglobulin G in which CH1 and CL of a single half-body are replaced with CD1a and β2m respectively.

“ABC174 format” or “174 format” used herein refers to a bispecific immunoglobulin G in which CH1 and CL of a single half-body are replaced with CD1b and β2m respectively.

The term “mutated”, “mutation” and “mutant” are interchangeably used herein. Typically, and preferably, said a mutated amino acid or a mutation is an exchange of one amino acid by one or more amino acids, an insertion, a deletion or a combination thereof. Most preferably, said a mutated amino acid or mutation is an exchange of a single amino acid by a different single amino acid.

### EXAMPLES

The amino acid sequences of SEQ ID Nos: 1-47, 57-89 and 101-111 are listed as follows.

| SEQ | |
|---|---|
| NO | Amino Acid Sequence |
| 1 | EIVLTQSPQTLSLSPGERATLSCRASQSYTSSYLAWYQQKPGQAPRLLINYGSRRATGIPDRFSGSGSGTDFTLTISRLEPEDFAVYYCQQG |
| | IMLPPTFGQGTKVEIKRTVAAPSVFIFPPSDEQLKSGIASVVCLLNNFYPREAKVQWKVDNALQSGNSQESVTEQDSKDSIYSLSSTLTL |
| | SKADYEKHKVYACEVTHQGLSSPVTKSFNRGEC |
| 2 | EVQLLESGGGLVQPGGSLRLSCAASGFTFSSYAMSWVRQAPGKGLEWVSAISGSGGSTYYADSVKGRFTISRDNSKNTLYLQMNSLRA |
| | EDTAVYYCAKGWFGGFNYWGQGTLVTVSSASTKGPSVEPLAPSSKSTSGGTAALGCLVKDYFPEPVTVSWNSGALTSGVHTFPAVLQS |
| | SGLYSLSSVVTVPSSSLGTQTYICNVNHKPSNTKVDKKVEPKSCDKTHTCPPCPAPELLGGPSVFLFPPKPKDTLMISRTPEVICVVVDV |
| | SHIEDPEVKENWYVDGVEVIINAKTKPREEQYNSTYRVVSVLTVLIIQDWLNGKEYKCKVSNKALPAPIEKTISKAKGQPREPQVYTLPP |
| | CREEMTKNQVSLWCLVKGFYPSDIAVEWESNGQPENNYKTIPPVLDSDGSFFLYSKLTVDKSRWQQGNVESCSVMHEALHNHYTQKS |
| | LSLSPGK |
| 3 | EVQLLESGGGLVQPGGSLRLSCAASGFTFSSYAMSWVRQAPGKGLEWVSAISGSGGSTYYADSVKGRFTISRDNSKNTLYLQMNSLRA |
| | EDTAVYYCAKGWFGGFNYWGQGTLVTVSSASTKGPSVFPLAPSSKSTSGGTAALGCLVKDYFPEPVTVSWNSGALTSGVIITFPAVLQS |
| | SGLYSLSSVVTVPSSSLGTQTYICNVNHKPSNTKVDKKVEPKSCDKTHTCPPCPAPELLGGPSVFLEPPKPKDTLMISRTPEVTCVVVDV |
| | SHHDPHVKENWYVDGVEVHNAKTKPRHEQYASTYRVVSVLTVLHQDWLNGKEYKCKVSNKALPAPIEKTISKAKGQPREPQVYTLPP |
| | CREEMTKNQVSLWCLVKGEYPSDIAVEWESNGQPENNYKTTPPVLDSDGSEFLYSKLTVDKSRWQQGNVFSCSVMHEALHNHYTQKS |
| | LSLSPGK |
| 4 | EIVLTQSPGTESLSPGERATLSGRASQSVISSYLAWYQQKPGQAPRILINVGSRRATGIPDRESGSGSGTDFTLTISRLEPEDFAVYYCQQG |
| | IMLPPTFGQGTKVEIKRTVAAPSVFIFPPSDEQLKSGTASVVCLLNNFYPREAKVQWKVDNALQSGNSQESVTEQDSKDSTYSLSSTLTL |
| | SKADYEKIIKVYACEVTIIQGLSSPVTKSFNRGECGLVPRGSMSDSEVNQEAKPEVKPEVKPETHINLKVSDGSSEIFFKIKKTTPLRRLM |
| | EAFAKRQGKEMDSLRFLYDGIRIQADQTPEDLDMEDNDIIEAHREQIGG |
| 5 | DIQMTQSPSSLSASVGDRVTITCRASQDVNTAVAWYQQKPGKAPKLLIYSASFLYSGVPSRFSGSRSGTDFTLTISSLQPEDFATYYCQQH |
| | YTTPPTFGQGTKVEIKRTVAAPSVFIFPPSDEQLKSGTASVVCLLNNFYPREAKVQWKVDNALQSGNSQESVTEQDSKDSTYSLSSTLTL |
| | SKADYEKHKVYACEVTHQGLSSPVTKSFNRGEC |
| 6 | EVQLVESQGGLVQPGGSLRLSCAASGFNIKDTYIIIWVRQAPGKGLEWVARTYPINGYTRYADSVKGRFTISADTSKNTAYLQMNSLRA |
| | EDTAVYYCSRWGGDGFYAMDYWGQGTLVTVSSASTKGPSVFPLAPSSKSTSGGTAALGCLVKDYFPEPVTVSWNSGALTSGVHTFPAY |
| | LQSSGLYSLSSVVTVPSSSLGTQTYICNVNHKPSNTKVDKKVEPKSCDKTHTCPPCPAPELLGGPSVFLFFPPKPKDTLMISRTPEVTCVVV |
| | DVSHEDPEVKFNWYVIGVEVHNAKTKPREEQYASTYRVVSVLTVEHQDWLMGIKHYKCKVSNKALPAPIEKTISKAKGQPREPQVCTL |
| | PPSREEMTKNQVSLSCAVKGFYPSDIAVEWESNGQPENNYKITPPVLDSDGSFFLVSKLTVDKSRWQQGNVFSCSVMHILALHNIIYTQK |
| | SLSLSPGK |
| 7 | EVQLVESGGGLVQPGGSLRLSCAASGENIKDTYIHWVRQAPGKGLEWVARTYPINGYTRYADSVKGRFTISADTSKNTAYLQMNSLRA |
| | EDTAVYYCSRWGGDGFYAMDYWGQGTLVTVSSGQPREPQVYTLPPCREEMTKNQVSLWCLVKGFYPSDIAVEWESNGQPENNYKTI |
| | PPVLDSDGSFFLYSKLTVDKSRWQQGKVFSCSVMHEALHNHYTQKSLSLSPGDKTHTCPPCPAPELLGGPSVFLFPPKPKDTLMISRTPE |
| | VTCVVYDVSHEDPEVKENWYVDGVEVHNAKTKPREEQYASTYRVVSVLIVLHQDWLNGKEYKCKVSNKALPAPIEKTISKAKGQPR |
| | EPQVCTIPPSREEMTKNQVSUSCAVKGFYPSDIAVEWESNGQPENNYKTTPPVLDSDGSFELVSKLTVDKSRWQQGNVESCSVMHEAL |
| | HNHYTQKSLSESPGK |
| 8 | EVQLVESGGGLVQPGGSLRLSCAASGENIKDTYIHWVRQAPGKGLEWVARITYPTKGYTRYADSVKGRFTISADTSKNTAYLQMNSLRA |
| | LDTAVYYCSRWGGDGFYAMDYWGQGTLVTVSSGQPREPQVCTLPPSREEMTKNQVSLSCAVKGFYPSDIAVEWESNGQPENNYKTTP |
| | PVLDSDGSFFLVSKLTVDKSRWQQGNVFSCSVMHEALHNHYTQKSLSLSPGDKTHTCPPCPAPELLGGPSVFLFPPKPKDTLMISRTPEV |
| | TCVVVDVSHEDPEVKENWYVDGVEVHNAKTKPREEQYASTYRVVSVLIVLHQDWLNGKEYKCKVSNKALPAPIEKTISKAKGQPREP |
| | QVCTLPPSREEMTKNQVSLSCAVKGFYPSDIAVEWESNGQPENNYKTTPPVLDSDGSFFLVSKLTVDKSRWQQGNVESCSVMHEALHN |
| | HYTQKSLSLSPGK |
| 9 | DIQMTQSPSSLSASVGDRVTITCRASQDVNIAVAWYQQKPGKAPKLLIYSASFLYSGVPSRFSGSRSGTDFTLTISSLQPEDFATYYCQQH |
| | YTTPPTFGQGTKVEIKRTPREPQVCTLPPSREEMTKNQVSLSCAVKGFYPSDIAVEWESNGQPENNYKTTPPVLDSDGSFFLVSKLTVDK |
| | SRWQQGNVFSCSVMHEALHNITYTQKSLSLSPG |
| 10 | DIQMTQSPSSLSASVGDRVTITCRASQDVNIAVAWYQQKPGKAPKLLIYSASFLYSGVPSRESGSRSGTDFTLTISSLQPEDFATYYCQQH |
| | YTTPPTFGQGTKVEIKRTPREPQVYTLPPCREEMTKNQVSLWCLVKGFYPSDIAVEWESNGQPENNYKTTPPVLDSDGISFFLYSKLTVD |
| | KSRWQQGNVFSCSVMHEALHNHYTQKSLSLSPG |
| 11 | DIQMTQSPSSLSASVGDRVTITCRASQDVNTAVAWYQQKPGKAPKLLIYSASFLYSGVPSRFSGSRSGTDFTLTISSLQPEDFATYYCQQH |
| | YTTPPTFGQGIKVEIKRTIQRTPKIQVYSRHPAENGKSNFLNCYVSGFHPSDIEVDLLKNGERIEKVEHSDLSFSKDWSFYLLYYTEFTPT |
| | EKDEYACRVNIIVTLSQPKIVKWDRDM |
| 12 | DIQMTQSPSSLSASVGDRVTITGRASQDVNTAVAWYQQKPGKAPKLLIYSASFLYSGVPSRESGSRSGTDFTLTISSLQPEDFATYYCQQH |
| | YTTPPTFGQGTKVEIKRTVAAPKTHMTHHAVSDHEATLRCWALSFYPAFITLTWQRDGEDQTQDTEJVETRPAGDGTFQKWAAVVVPS |
| | GQEQRYTCHVQHEGLPKPLTLRWELSS |
| 13 | EVQLVESGGGIVQPGGSIRLSCAASGENIKDTYIHWVRQAPGKGLEWVARIYPINGYTRYADSVKGRETISADTSKNTAYLQMNSLRA |
| | EDTAVYYCSRWGGDGFYAMDYWGQGTLVTVSSASTKGRIDAPKTHMTHHAVSDHEATLRCWALSFYPAEITLTWQRDGEDQTQDTE |
| | LVETRPAGDGTFQKWAAVVVPSGQEQRYTCHVQHEGLPKPLTLRWELSSDKTHTCPPCPAPELLGGPSVFLFPPKPKDILMISRTPEVTQ |
| | VVVDVSHEDPEVKENWYVDGVEVHNAKTKPREEQYASTYRVVSVLTVLHQDWLNGKEYKCKVSNKALPAPIEKTISKAKGQPREPQ |
| | VCTIPPSRHEMTKNQVSLSCAVKGFYPSDIAVEWESNGQPENNYKTTPPVLDSDGSFELVSKITVQKSRWQQGNVESCSVMHEATHNH |
| | YTQKSISLSPGK |
| 14 | EVQLVESGGGLVQPGGSLRLSCAASGFNIKDYIHWVRQAPGKGLEWVARIYPTKGYTRYADSVKGRFTISADISKNTAYLQMNSLRA |
| | EDTAVYYCSRWGGDGFYAMDYWGQGILVTVSSASTKGIQRTPKIQVYSRHPAENGKSNELNCYVSGFHPSDIEVDLLKNGERIEKVEH |
| | SDLSFSKDWSFYLLYYTEFTPTEKDEYACRVNHVTLSQPKIVKWDRDMGSDKTHTCPPCPAPELLGGPSVFLFPPKPKDILMISRTPEVT |
| | CVVVDVSIIEDPEVKENWYVDGVEVIINAKIKPREEQYASTYRVVSVLTVLIIQDWLNGKEYKCKVSNKALPAPIEKTISKAKGQPREP |
| | QVCTLPPSREEMTKNQVSLSCAVKGFYPSDIAVEWESNGQPENNYKTTPPVLDSDGSFFLVSKLTVDKSRWQQGNVFSCSVMHEALHN |
| | HYTQKSLSLSPGK |
| 15 | EVQLVESGGGLVQPGGSLRLSCAASGFNIKDTYIHWVRQAPGKGLEWVARIYPTNGYTRYADSVKGRFTISADISKNTAYLQMNSLRA |
| | EDTAVYYCSRWGGDGFYAMDYWGQGTLVTVSSASTKLRLMAPISLQVVHVETHRCNISWEISQASHYFERHLEFEARTLSPGHTWEE |
| | APLLTLKQKQEWICLETLTPDTQYEFQVRVKPLQGEFTTWSPWSQPLAFRTKPDKTIITCPPCPAPELLQGPSVFLFPPKPKDTLMISRTPE |
| | VTCVVVDVSIIEDPEVKFNWYVDGVEVIINAKTKPREEQYASTYRVVSVLTVLIIQDWLNGKEYKCKVSNKALPAPIEKTISKAKGQPR |
| | EPQVCTLPPSREEMTKNQVSLSCAVKGFYPSDLAVEWESNGQPENNYKTIPPVLDSDGSFFLVSKLTVDKSRWQQGNVFSCSVMHEAL |
| | ENHYTQKSLSLSPGK |
| 16 | DIQMTQSPSSLSASVGDRVTITCRASQDVNTAVAWYQQKPGKAPKILIYSASFLYSGVPSRESGSRSGTDETLTISSLQPEDFATYYCQQH |
| | YTTPPTFGQGTKVEIKRTSSASLVIPWAPENLTLHKLSESQLELNWNNRFLNHCLEHLVQYRTDWDHSWTEQSVDYRHKFSLPSVDGQ |
| | KRYTFRVRSRFNPLCGSAQHWSEWSHPIHWGSNTS |
| 17 | EVQLVESGGGLVQPGGSLRESCAASGFNIKDTYIHWVRQAPGKGLEWVARIYPINGYTRYADSVKGRATISADTSKNTAYLQMNSLRA |
| | EDIAVYYCSRWGGDGFYAMDYWGQGTLVTVSSASTKGSRRELTDTLQAETDQLEDEKSALQTEIANLLKEKEKLEFILADKTHTCPPC |
| | PAPRIKAPSVFLFPPKPKDTLMISRTPEVTCVVVDVSHEDPEVKFNWYVDGVEVHNAKTKPREEQYASTYRVVSVLTVLHQDWING |
| | KEYKCKVSNKALPAPIEKTISKAKGQPREPQVCTLPPSREEMTKNQVSLSCAVKGFYPSDIAVEWESNGQPENNYKTTPPVLDSDGSEFL |
| | VSKITVDKSRWQQGNVFSCSVMHEALHNHYTQKSISLSPGK |
| 18 | DIQMTQSPSSLSASVGDRVTITCRASQDVNTAVAWYQQKPGKAPKLLIYSASFLYSGVPSRFSGSRSGTDFTLTISSLQPEDFATYYCQQH |
| | YTTPPTFGQGTKVEIKRTVAASKLERIARLEEKVKILKAQNSELASTANMLREQVAQLKQKVM |
| 19 | EVQLVESGGGLVQPGGSLRLSCAASGFNIKDTYIIIWVRQAPGKGLEWVARTYPINGYTRYADSVKGRFTISADTSKNTAYLQMNSLRA |
| | EDTAVYYCSRWGGDGFYAMDYWGQGTLVTVEEELQVIQPDKSVSVAAGESAILIICTVTSLIPVGPIQWERGAGPARELIYNQKIGIIFP |
| | RVTTVSESTKRENMDFSISISNITPADAGIYYCVKFRKGSPDTLEKSGAGTELSVRAKPSTRDKTHICPPCPAPELLGGPSVELFPPKPKD |
| | TLMISRTPEVTCVVVDVSHEDPEVKENWYVDGVEVHNAKIKPREEQYASTYRVVSVLTVLHQDWLNGKEYKCKVSNKALPAPIEKTI |
| | SKAKGQPREPQVCTLPPSREEMTKNQVSLSCAVKGFYPSDIAVEWESNGQPENNYKTTPPVLDSDGSFFLVSKLIVDKSRWQQGNVFS |
| | CSVMHEALHNHYTQKSLSLSPGK |
| 20 | DIQMTQSPSSLSASVGDRVTITCRASQDVNTAVAWYQQKPGKAPKLLIYSASFLYSGVPSRFSGSRSGTDFTLTISSLQPEDFATYYCQQH |
| | YTTPPTEGQGTKVEIKQLLFNKTKSVEFTFGNDTVVIPCFVTNMEAQNTTEVYVKWKFKGRDIYTFDGALNKSTVPTDESSAKIEVSQL |
| | LKGDASLKMDKSDAVSHITGNYTCEVTELTREGETHIELKYRVVSWSTR |
| 21 | DIQMTQSPSSLSASVGDRVTITCRASQDVNTAVAWYQQKPGKAPKLLIYSASFLYSGVPSRFSGSRSGTDFTLTISSLQPEDFATYYCQQH |
| | YTTPPTFGQGTKVEIKRTVAASGGGGGGGGSGGGGSGGGGTSTNNNEEEKSKLLEKENRELEKIIAEKEERVSELRHQLQSR |
| 22 | EVQLVESGGGLVQPGGSLRLSCAASGENIKDTYIHWVRQAPGKGLEWVARIYPINGYTRYADSVKGRFTISADTSKNTAYLQMNSLRA |
| | EDTAVYYCSRWGGDGEYAMDYWGQGTLVTVSSASTKGSGGGGSGGGGSGGGGGGGGTSYNQASTSRLEGLQSENHHLRMKITELD |
| | KDLEEVTMQLQDTDKTHTCPPCPAPELLGGPSVFLFPPKPKDTLMISRTPEVTCVVVDVSHEDPEVKENWYVDGVEVHNAKTKPREEQ |
| | YASTYRVVSVITVLHQDWINGKEYKCKVSNKALPAPIRKTISKAKGQPREPQVCTLPPSREEMTKNQVSLSCAVKGFYPSDIAVEWHSN |
| | GQPENNYKTTPPVLDSDGSEFLVSKLTVDKSRWQQGNVESCSVMHRALHNHYTQKSLSLSPGK |
| 23 | MGWSCIILFLVATATGVHSDIQMTQSPSSLSASVGDRVTITCRASQDVNTAVAWYQQKPGKAPKLLIYSASFLYSGVPSRFSGSRSGTDET |
| | LTISSLQPEDFATYYCQQHYTTPPTFGQGTKVEIKRTVAASGGGGSGGGGSGGGGSGGGGTSVNQASTSRLEGLQSENHHLRMKITELD |
| | KDLEEVTMQLQDT |
| 24 | EVQLVESGGGLVQPGGSLRLSCAASGFNIKDTYIHWVRQAPGKGLEWVARIYPTNGYTRYADSVKGRFTISADTSKNTAYLQMNSLRA |
| | EDTAVYYCSRWGGDGFYAMDYWGQGTLVTVSSASTKGSGGGGSGGGGSGGGGSGGGGTSTNNEEEKSRLLEKENRLLEKIIAEKEE |
| | RVSELRIIQLQSRDKTITTCPPCPAPELLGGPSVELFPPKPKDTLMISRTPEVTCVVVDVSHIEDPEVKFNWYVDGVEVIINAKIKPREEQY |
| | ASTYRVVSVLTVLHQDWLNGKEYKCKVSNKALPAPIEKTISKAKGQPREPQVCTLPPSREEMTKNQVSLSCAVKGFYPSDIAVEWESN |
| | GQPENNYKTIPPVLDSDGSFFLVSKLTVDKSRWQQGNVFSCSVMHEALHNHYTQKSLSLSPQK |
| 25 | DIQMTQSPSSLSASVGDRVTITCRASQDVNTAVAWYQQKPGKAPKLLIYSASFLYSGVPSRESGSRSGTDFTLTISSLQPEDFATYYCQQH |
| | YTTPPTFGQGTKVEIKRTVAQVKPEAWLSHGPSPGPGHLQLVCHVSGFYPKPVWVMWMRGEQEQQGTQRGDILPSADGTWYLRATLE |
| | VAAGEAADLSCRVKHISSLEGQDIVLYWEGSLVPRG |
| 26 | DIQMTQSPSSLSASVGDRVTITCRASQDVNTAVAWYQQKPGKAPKLLIYSASFLYSGVPSRFSGSRSGTDFTLTISSLQPEDEATYYCQQH |
| | YTTPPTFGQGTKVEIKRTVAQVKPFAWLSSGPSPGPGRLQIVCHVSGFYPKPVWVMWMRGFQFQQGTQLGDILPKANWTWYLRATL |
| | DVADGEAAGLSCRVKASSLEGQDIILYWRGSGLNDIFFAQKIEWHE |
| 27 | EVQLVESGGGLVQPGGSLRLSCAASGENIKDTYIHWVRQAPGKGLEWVARIYPINGYIRYADSVKGRFTISADISKNTAYLQMNSERA |
| | EDTAVYYCSRWGGDGEYAMDYWGQGTLVTVSSASTKGQVKPEAWLSHGPSPGPGHLQLVCHVSGFYPKPVWVMWMRGEQEQQGT |
| | QRGDILPSADGTWYLRATLEVAAGEAADLSCRVKHSSLEGQDIVLYWEGSLVPRGDKTHTCPPCPAPELLGGPSVFLFPPKPKDILMISR |
| | TPHVTCVVVDVSHEDPEVKENWYVDGVEVHNAKTKPREHQYASTYRVVSVLTVLHQDWINGKHYKCKVSVKALPAPIEKTISKAKG |
| | QPREPQVCTLPPSREEMTKNQVSLSCAVKGFYPSDIAVEWESNGQPENNYKITPPVLDSDGSFFLYSKITVDKSRWQQGNVFSCSVMH |
| | EALHNHYTQKSLSLSPGK |
| 28 | EVQLVESGGGLVQPGGSLRLSCAASGFNIKDTYIIIWVRQAPGKGLEWVARTYPINGYTRYADSVKGRFTISADTSKNTAYLQMNSLRA |
| | EDTAVYYCSRWGGDGFYAMDYWGQGTLVTVSSASTKGSGGGGSGGGGSGGGGGGGGTLQQTPAYIKVQTNKMVMLSCEAKISLSN |
| | MRIYWLRQRQAPSSDSHHEELALWDSAKGTIHGEEVEQEKIAVFRDASRFILNLTSVKPEDSGIYFCMIVGSPELTFGKGTQLSVVDEDK |
| | THTCPPCPAPELLGGPSVFLFPPKPKDTLMISRTPEVTCVVVDYSHEDPEVKFNWYVDGVEVHNAKTKPREEQYASTYRVVSVLTVLHQ |
| | DWLNGKEYKCKYSNKALPAPIEKTISKAKGQPREPQVCTLPPSREEMTKNQVSLSCAVKGFYPSDIAYEWESNGQPENNYKITPPVLDS |
| | DGSFFLVSKLTVDKSRWQQINVFSCSVMHEALHNHYTQKSISLSPGK |
| 29 | DIQMTQSPSSLSASVGDRVTITCRASQDVNTAVAWYQQKPGKAPKLLIYSASFLYSGVPSRFSGSRSGTDFTLTISSLQPEDFATYYCQQH |
| | YTTPPTFCQGTKVEIKRTVAASGGGGSGGGGSQGGGSGGGGTPSQFRVSPLDRTWNLGETVELKCQVLLSNPTSGCSWLFQPRGAAAS |
| | PIELLYLSQNKPKAAEGLDTQRFSGKRLGDTFVLTLSDERRENEGYYFCSALSNSIMYFSHIFVPVFLP |
| 30 | EVQIVESGGGLVQPGGSLRISCAASGENIKDTYTHWVRQAPGKGLEWVARTYPINGYTRYADSVKGRFTISADTSKNTAYLQMNSLRA |
| | EDTAVYYCSRWGGDGFYAMDYWGQGTLVTVSSASTKGSGGGGSGGGGSGGGGSGGGGTLQQTPAYIKVQTNKMVMLSCEAKISLSN |
| | MRIYWLRQRQAPSSDSHHEFLALWDSAKGTIHGEEVEQEKIAVFRDASRFILNLTSVKPEDSGIYFCMIVGSPELTFGKGTQLSVVDEDK |
| | THTCPPCPAPELLGGPSVFLFPPKPKDTLMISRTPEVTCVVVDVSHEDPEVKENWYVDGVEVHNAKTKPREEQYASTYRVVSVLIVLHQ |
| | DWLNGKEYKCKVSNKALPAPIEKTISKAKGQPREPQVCTLPPSREEMTKNQVSLSCAVKGFYPSDIAVEWESNGQPENNYKTTPPVLDS |
| | DGSFFLVSKLTVDKSRWQQGNVFSCSVMHEALHNHYTQKSLSLSPGK |
| 31 | EVQLVESGGGLVQPGGSLRLSCAASGFNIKDTYIITWVRQAPGKGLEWVARTYPINGYTRYADSVKGRFTISADTSKNTAYLQMNSERA |
| | EDTAVYYCSRWGGDGFYAMDYWGQGTLVTVSSASTKGPSVFPLAPSSKSTSGGTAALGCLVKDYFPEPVTVSWNSGALISGVHIFPAV |
| | LQSSGLYSLSSVVTVPSSSLQTQTYICKVNIIKPSKTKVDKKVIPKSCDKTIITCPPCPAPELLGGPSVFLFPPKPKDILMISRTPEVTCVVV |
| | DVSHEDPEVKFNWYVDGVEVHNAKTKPREEQYNSTYRVVSVLTVLHQDWLNGKEYKCKVSNKALPAPIEKTISKAKGQPREPQVYTL |
| | PPSREEMTKNQVSLTCLVKGFYPSDIAVEWESNGQPENNYKTTPPVLDSDGSFFLYSKLTVDKSRWQQGKVFSCSVMHEALHNIIYTQK |
| | SLSLSPGK |
| 32 | EVQLVESGGGLVQPGGSLRLSCAASGFNIKDTYTHWVRQAPGKGLEWVARTYPINGYTRYADSVKGRFTISADTSKNTAYLQMNSERA |
| | EDTAVYYCSRWGGDGFYAMDYWGQGTLVTVSSASVAAPSVEJEPPSDEQLKSGTASVVCLENNFYPREAKVQWKVDNALQSGNSQES |
| | VTEQDSKDSTYSLSSTETISKADYEKHKVYACEVTHQGLSSPVTKSENRGECDKTHTCPPCPAPELLGGPSVFLFPPKPKDTLMISRTPE |
| | VTCVVVDVSHEDPEVKFNWYVDGVEVHNAKTKPREEQYASTYRVVSVLTVLHQDWLNGKEYKCKVSNKALPAPIEKTISKAKGQPR |
| | EPQVCTLPPSREEMTKKQVSLSCAVKGFYPSDIAVEWESNGQPENNYKTIPPVLDSDGSFFLVSKLTVDKSRWQQGNVFSCSVMHEAL |
| | IINIYTQKSLSLSPGK |
| 33 | DIQMTQSPSSLSASVGDRVTITCRASQDVNIAVAWYQQKPGKAPKLLIYSASFLYSGVPSRFSGSRSGTDFTLTISSLQPEDFATYYCQQH |
| | YTTPPTFGQGTKVEIKSSASTKGPSVFPLAPSSKSTSGGTAALGQLVKDYEPEPVTVSWNSGALTSGVHTFPAVLQSSGLYSLSSVVTVPS |
| | SSLGTQTYICNVNHKPSNTKVDKKVEPKSC |
| 34 | EVQLVESGGGLVQPGGSLRLSCAASGENIKDTYIHWVRQAPGKGLEWVARIYPINGYTRYADSVKGRFTISADISKNTAYLQMNSLRA |
| | EDTAVYYCSRWGGDGFYAMDYWGQGTLVIVLEDLKNVFPPEVAVFEPSEAEISHTQKATLVCLATGFYPDHVELSWWVNGKEVHSGV |
| | CTDPQPLKEQPALQDSRYALSSRLRVSATFWQNPRNIIFRCQVQFYGLSENDEWTQDRAKPVTQIVSAEAWGRASDKTIITCPPCPAPEL |
| | LGGPSVFLFPPKPKDTLMISRTPEVTCVVVDVSIIEDPEVKENWYVDGVEVIINAKTKPREEQYASTYRVVSVLIVLIIQDWLNGKEYKC |
| | KVSKKALPAPIEKTISKAKGQPREPQVCTLPPSREEMTKNQVSLSCAVKGFYPSDIAVEWESNGQPENNYKTTPPVLDSDGSFFLVSKLT |
| | VDKSRWQQGNVFSCSVMHEALHNHYTQKSLSLSPGK |
| 35 | DIQMTQSPSSLSASVGDRVTITCRASQDVNTAVAWYQQKPGKAPKLLIYSASFLYSGVPSRFSGSRSGTDFTLTISSLQPEDFATYYCQQH |
| | YTTPPTFGQGIKVEIKPDIQNPDPAVYQLRDSKSSDKSVCLFTDFDSQTQVSQSKDSDVYITDKCVLDMRSMDFKSNSAVAWSQKSDFA |
| | CANAFQNSHIPEDTFFPSPESS |
| 36 | EVQLVESGGGLYQPGGSLRLSCAASGEPFNTFAMNWVRQAPGKGLEWLARIRSKSNNYATYYADSVKDRETISRDNAKRSLYLQMNSL |
| | RAHDTAVYYGVRGKENNYGYAMDYWGQGTTVTVSSASTKGQVKPRAWLSHGPSPGPGHLQIVCHVSGFYPKPVWVMWMRGEQEQ |
| | QGTQRGDILPSADGTWYLRATLEVAAGEAADLSCRVKHSSLEGQDIVLYWEGSLVPRGDKTHTCPPCPAPELLGGPSVFLFPPKPKDTL |
| | MISRTPEVTCVVVDVSHEDPEVKENWYVDGVEVHNAKTKPREFQYASTYRVVSVLTVLHQDWLNGKEYKCKVSNKALPAPJEKTISK |
| | AKGQPREPQVCTIPPSREEMTKNQVSLSCAVKGFYPSDIAVEWESNGQPENNYKTTPPVLDSDGSFFLVSKETVDKSRWQQGNVFSCS |
| | VMHEALHNHYTQKSLSLSPGK |
| 37 | DIVMIQSPLSLPVTPGEPASISCRSSKSLLIISNANTYLYWFLQKPGQSPQLLIYRMSNLASGVPDRFSGSGSGTDFTLKISRVEAEDVGVY |
| | YCMQHVEYPFIFGGGIKLEIKRTIQRTPKIQVYSRHPAENGKSNFLNCYVSGEHPSDIEVDLLKNGERIEKVEHSDLSFSKDWSFYLLYY |
| | TEFIPTEKDEYACRVNHVILSQPKIVKWDRDM |
| 38 | QVQLQQPGAELVRPGTSVKLSCKASGYTFTSYWMHWIKQRPGQGLEWIGVIDPSDNYTKYNQKFKGKATLTVDTSSSTAYMQLSSLTS |
| | EDSAVYFCAREGYYGSSPWFAYWGQGTLVTVSSASTKGQVKPEAWLSHGPSPGPGHLQLVCHVSGFYPKPVWVMWMRGEQEQQGT |
| | QRGDILPSADGTWYLRATLEVAAGEAADLSCRVKIISSLEQQDIVLYWIGSLVPRGDKTIITCPPCPAPELLGGPSVELFPPKPKDTLMISR |
| | TPEVTCVVVDVSIIEDPEVKENWYVDGVEVIIKAKTKPREEQYASTYRVVSVLTVLIIQDWLNGKEYKCKVSNKALPAPIEKTISKAKG |
| | QPREPQVCTLPPSREEMTKNQVSLSCAVKGFYPSDIAVEWESNGQPENNYKTIPPVLDSDGSFFLVSKLTVDKSRWQQGNVFSCSVMH |
| | EALHNHYTQKSLSLSPGK |
| 39 | QVQLQQPGAELVRPGTSVKLSCKASGYTFTSYWMHWIKQRPGQGLEWIGVIDPSDKYTKYNQKFKGKATLTVDTSSSTAYMQLSSLTS |
| | EDSAVYFCAREGYYGSSPWFAYWGQGTLVTVSSASVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSENRGECDKTHTCPPCPAPELLGGPSVELFPPKPKDTLMISRTPE |
| | VTCVVVDVSHEDPEVKENWYVDGVEVHNAKTKPRERQYASTYRVVSVLTVLHQDWINGKEYKCKVSNKALPAPIEKTISKAKGQPR |
| | EPQVCTLPPSREEMTKKQVSLSCAVKGFYPSDLAVEWESNGQPENNYKTTPPVLDSDGSFFLVSKLTVDKSRWQQGNVFSCSVMHEAL |
| | HNHYTQKSLSLSPGK |
| 40 | QVQLQQPGAELVRPGTSVKLSCKASGYTFTSYWMHWIKQRPGQGLEWIGVIDPSDNYTKYNQKFKGKATLTVDTSSSTAYMQLSSLTS |
| | EDSAVYFCAREGYYGSSPWFAYWGQGTLVTVSSLEDLKNVFPPEVAVFEPSEAFISHTQKATLVCLATGEYPDHVELSWWVNGKEVHS |
| | GVCTDPQPLKEQPALQDSRYALSSRLRVSATFWQNPRNHFRCQVQFYGLSENDEWTQDRAKPVTQIVSAEAWGRASDKTHTCPPCPAP |
| | ELIGGPSVELFPPKPKDTEMISRTPEVTCVVVDVSHEDPEVKENWYVDGIVEVHNAKTKPRREQYASTYRVVSVLTVLHQDWINGKEY |
| | KCKVSNKALPAPIEKTISKAKGQPREPQVCTLPPSREEMTKNQVSLSCAVKGFYPSDIAVEWESNGQPENNYKTTPPVLDSDGSFFLVSK |
| | LTVDKSRWQQQNVFSCSVMITEALHNITYTQKSLSLSPQK |
| 41 | QIVMSQSPAILSASPGEKVTMTCRARSSVSYIHWYQQKPGSSPKPWIYATSNLASGVPGRFSGSGSGISYSLTITRVEAEDAATYYCQQW |
| | SSKPPTFGGGTKLEIKRTIQRTPKIQVYSRIIPAENGKSNFLNCYVSGFHIPSDIEVDLLKNGERIEKVEIISDLSFSKDWSFYLLYYTE |
| | FTPTEKDEYACRVNIIVTLSQPKIVKWDRDM |
| 42 | QIVMSQSPAILSASPGEKVTMTCRARSSVSYIHWYQQKPGSSPKPWIYATENLASGVPGRESGSGSGTSYSLTITRVEAEDAATYYCQQW |
| | SSKPPTFGGGTKLEIKSSASTKGPSVFPLAPSSKSTSGGTAALGCLVKDYFREPVTVSWNSGALTSGVHTFPAVLQSSGLYSLSSVVTVPS |
| | SSLGTQTYICNVNIIKPSNTKVDKKVEPKSC |
| 43 | QIVMSQSPAILSASPGEKVTMTCRARSSVSYIIIWYQQKPGSSPKPWIYATSNLASGVPGRESGSGSGISYSLTITRVEAEDAATYYCQQW |
| | SSKPPTFGGGTKLEIKPDIQNPDPAVYQLRDSKSSDKSVCLFTDFDSQTQVSQSKDSDVYITDKCVLDMRSMDFKSNSAVAWSQKSDEA |
| | CANAFQNSIIPEDTFFPSPESS |
| 44 | DIQMTQSPSSLPASLGDRVTINCQASQDISNYLNWYQQKPGKAPKLLIYYINKLADGVPSRFSGSGSGRDSSFTISSLESEDIGSYYCQQ |
| | YYNYPWTFGPGTKLEIKRTVAAPSVFIFPPSDEQLKSGTASVVCLLNNFYPREAKVQWKVDNALQSGNSQESVTEQDSKDSTYSLSSTL |
| | TLSKADYEKHKVYACEVTHQGLSSPVTKSENRGEC |
| 45 | EVQLVESGGGLVQPGKSLKLSCEASGFTFSGYGMIIWVRQAPGRGLESVAYITSSSINIKYADAVKGRFTVSRDNAKNLLFLQMNILKSE |
| | DTAMYYCARFDWDKNYWGQGTMVTVSSASTKGPSVEPLAPSSKSTSGGTAALGCLVKDYFPEPVTVSWNSGALTSGVHTFPAVLQSS |
| | GLYSESSVVTVPSSSIGTQTYICNVNHKPSNTKVDKKVEPKSCQKTHTCPPCPAPELLGGPSVELFPPKPKDTIMISRTPEVTCVVVDVS |
| | HEDPEVKFNWYVDGVEVHNAKTKPREEQYASTYRVVSVLIVLHQDWLNGKEYKCKVSNKALPAPIEKTISKAKGQPREPQVYTLPPC |
| | REEMTKNQVSLWCLVKGFYPSDIAVEWESKGQPENNYKTTPPVLDSDGSFFLYSKLTVDKSRWQQGNVFSCSVMHEALHNHYTQKSL |
| | SLSPGK |
| 46 | AIQMTQSPSSLSASVGDRVTITCRASQDIRNYLNWYQQKPGKAPKLLIYYTSRLESGVPSRESGSGSGTDYTLTISSLQPEDFATYYCQQ |
| | GNTLPWTFGQGTKVEIKRTVAAPSVFIFPPSDEQLKSGTASVVCLLNNFYPREAKVQWKVDNALQSGNSQESVTEQDSKDSTYSLSSTL |
| | TLSKADYEKHKVYACEVTHQGLSSPVTKSENRGEC |
| 47 | EVQLVESGGGLVQPGGSLRLSCAASGYSFTGYTMNWVRQAPGKGLEWVALINPYKGVSTYNQKFKDRFTISVDKSKNTAYLQMNSLR |
| | AEDTAVYYCARSGYYQDSDWYFDVWCQGTLVTVSSASTKGPSVFPLAPSSKSTSGGTAALGCLVKDYFPEPVTVSWNSGALTSQVIIT |
| | FPAVLQSSGLYSLSSVVTVPSSSLGTQTYICNVNHKPSNTKVDKKVEPKSCDKTHTCPPCPAPELLGGPSVFLFPPKPKDILMISRTPEVT |
| | QVYTLPPCREEMTKNQVSLWCLVKGFYPSDLAVEWESNGQPENNYKTTPPVLDSDGSFFLYSKLTVDKSRWQQGNVFSCSVMHEACH |
| | NIYTQKSLSLSPGK |
| 57 | DIVMTQSPLSLPVTPGEPASISCRSSKSLLHSNANTYLYWFLQKPGQSPQLLIYRMSNLASGVPDRFSGSGSGTDFILKISRVEAEDVGVY |
| | YCMQIIVEYPFTFGGGTKLEIKSSASTKGPSVFPLAPSSKSTSGGTAALGCLVKDYFPEPVTVSWNSGALTSGVIITFPAVLQSSGLYSLSS |
| | VVTVPSSSLGTQTYICNVNHKPSNTKVDKKVEPKSC |
| 58 | FVQIVESGGGLVQPGGSERLSCAASGEPFNTEAMNWVRQAPGKGLEWLARIRSKSNKYATYYADSVKDRFTISRDNAKRSLYLQMNSL |
| | RAEDTAVYYCVRGKENNYGYAMDYWGQGTTVTVSSASVAAPSVFIFPPSDEQLKSGTASVVCLLNNFYPREAKVQWKVDNALQSGN |
| | SQESVTEQDSKDSTYSLSSTLILSKADYEKIKVYACEVTIIQGLSSPVTKSFNRGECDKTHTCPPCPAPELLGGPSVELFPPKPKDTLMIS |
| | RTPEVTCVVYDVSHEDPEVKENWYVDGVEVHNAKTKPREEQYASTYRVYSVETVLHQDWINGKEYKCKVSNKALPAPIEKTISKAKG |
| | QPREPQVCTIPPSRHEMTKNQVSLSCAVKGFYPSDIAVEWESNGQPENNYKTTPPVLDSIXGASHELVSKETVDKSRWQQGINVESCSVMH |
| | EALHNHYTQKSLSLSPGK |
| 59 | EVQLVESGGGLVQPGGSLRLSCAASGENIKDTYIHWVRQAPGKGLEWVARTYPINGYTRYADSVKGRETISADTSKNTAYLQMNSERA |
| | EDTAVYYCSRWGGDGFYAMDYWGQGTLVTVSSASTKGQVKPEAWLSSCPSPGPGRLQLVCHVSGFYPKPVWVMWMRGEQEQQGTQLSEI |
| | LPNANWTWYLRATLDVADGEAAGLSCRVKIISSLEGQDIILYWRGSGDKTIITCPPCPAPELLGGPSVFLFPPKPKDTLMISRTPEVT |
| | CVVVDVSIIEDPEVKENWYVDGVEVIINAKTKPREEQYASTYRVVSVLIVLIIQDWLNGKEYKCKVSNKALPAPIEKTISKAKGQPREP |
| | QVCTIPPSREEMIKNQVSLSCAVKGFYPSDIAVEWESNGQPENNYKTIPPYLDSDGSFFLVSKLIVDKSRWQQGNVESCSVMHEALHN |
| | HYTQKSLSLSPQK |
| 60 | EIVMTQSPATLSVSPGERATLSCRASQSIGNNLIIWYQQKPGQAPRLLIYYASQSISGIPARFSGSGSGTEFTLTISSLQSEDFAVYYCQ |
| | QSNSWPYTFGGGTKVEIKRTVAAPSVFIFPPSDEQLKSGTASVVCLLNKFYPREAKVQWKVDNALQSGNSQESVTHQDSKQSTYSLSSTL |
| | TLSKADYEKHKVYACEVTHQGLASSPVTKSENRGEC |
| 61 | QVQLQESGPGLMKPSETLSLTCSVSGDSIRSDYWSWIRQPPGKGLEYIGYVSYSGSTYYNPSLKSRVTISVDTSKNRFSLKLNSVTAADT |
| | AVYYCARWDGDYWGQGILVTVSSASTKGPSVFPLAPSSKSTSGGIAALGCLVKDYFPEPVTVSWNSGALISGVHTFPAVLQSSGLYSLS |
| | SVVTVPSSSLGTQTYICNVNHKPSNTKVDKKVEPKSCDKTHTCPPCPAPELLGGPDVFLFPPKPKDTLMISRTPEVTCVVVDVSHEDPEV |
| | KFNWYVDGVEVHNAKTKPREEQYNSTYRVVSVLIVLHQDWLNGKEYKCKVSNKALPAPEEKTISKAKGQPREPQVYTLPPSREEMT |
| | KNQYSLTCLVKGFYPSDIAVEWESNGQPENNYKTTPPVLDSDGSFFLYSKLIVDKSRWQQGNVESCSVMHEALHNHYTQKSLSLSPGK |
| 62 | EVQLVESGGGLVQPGGSLRLSCAASGFPFNTEAMNWVRQAPGKGLEWLARIRSKSNNYATYYADSVKDRFTISRDNAKRSLYLQMKSL |
| | RAEDTAVYYCVRGKENNYGYAMDYWGQGTTVTVLEDLKNVEPPEVAVFEPSEAFISHTQKATIVCLATGFYPDHVELSWWVNGKEV |
| | HSGVCTDPQPLKEQPALQDSRYALSSRLRVSATFWQNPRNHFRCQVQFYGLSENDEWTQDRAKPVTQIVSAEAWGRASDKTHTCPPCP |
| | APELLGGPSVFLFPPKPKDTLMISRTPEVTCVVVDVSLIEDPEVKFNWYVDGVEVIINAKTKPREEQYASTYRVVSVLTVLIIQDWLNGK |
| | ZYKCKVSNKALPAPIEKTISKAKGQPREPQVCTLPPSREEMTKNQVSLSCAVKGFYPSDIAVEWESNGQPENNYKTTPPVLDSDGSFFLV |
| | SKLTVDKSRWQQGNVESCSVMHEALHNIIYTQKSLSLSPGK |
| 63 | EVQLLESGGGLVQPGGSLRLSCAASGFTFSSYAMSWVRQAPGKGLEWVSAISGSGGSTYYADSVKGRFTISRDNSKNTLYLQMNSLRA |
| | EDTAVYYCAKGWFGGENYWGQGTLVTVSSASTKGPSVFPLAPSSKSTSGGTAALGCLVKDYFPEPVTVSWNSGALISGVHTFPAVLQS |
| | SGLYSLSSVYTVPSSSLGTQTYICNVNHKPSNTKVDKKVEPKSCDKTHTCPPCPAPELLGGPSVELFPPKPKDILMISRTPEVTCVVVDV |
| | SHEDPEVKENWYVDGVEVHNAKTKPREEQYNSTYRVVSVLIVLHQDWLNGKEYKCKVSNKALPAPIEKTISKAKGQPREPQVYTLPP |
| | SRERMTKNQVSLTCLVKGFYPSDIAVEWESNGQPENNYKTTPPVIDSDGSEFLYSKETVDKSRWQQGNVFSCSVMHEALHNHYTQKSL |
| | SISPGK |
| 64 | EVQLLESGGGLVQPGGSLRLSCAASGFTFSSYAMSWVRQAPGKGLEWVSAISGSGGSTYYADSVKGRFTISRDNSKNTLYLQMNSLRA |
| | EDTAVYYCAKGWFGGENYWGQGTLVTVSSASTKGPSVFPLAPSSKSTSGGTAALGCLVKDYFPEPVTVSWNSGALTSGVHTFPAVLQS |
| | SGLYSLSSVVTVPSSSLGTQTYICNVNIIKPSNTKVDKKVEPKSCDKTIITCPPCPAPELLGGPSVFLFPPKPKDTLMISRTPEVTCVVVDV |
| | SIIEDPEVKFNWYVDGVEVIINAKTKPREEQYASTYRVVSVLIVLIIQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTVDKSRWQQGNVESCSVMHEALHNITYTQKSL |
| | SLSPGK |
| 65 | DIQMTQSPSSLSASVGDRYTITCRASQDVNTAVAWYQQKPGKAPKLLJYSASFLYSGVPSRESGSRSGTDETLTISSLQPRDEATYYCQQH |
| | YTTPPTFGQGTKVEIKRTIQRTPKIQVYSCHPAENGKSNFLNCYVSGFHPSDIEVDLLKNGERIEKVEHSDLSFSKDWSFYLLYYTEETPT |
| | EKDEYACRVNHVTLSQPKIVKWDRDM |
| 66 | EVQLVESGGGLVQPGGSLRLSCAASGENIKDTYIIIWVRQAPGKGLEWVARIYPINGYTRYADSVKGRFTISADTSKNIAYLQMNSLRA |
| | EDTAVYYCSRWGGDGFYAMDYWGQGTLVTVSSASTKGQVKPEAWLSHGPSPGPGHLQLVCHVSGFYPKPVWVMWMRGEQEQQGT |
| | QRGDILPSACGTWYLRATLEVAAGEAADLSCRVKHSSLEGQDIVIYWEGSLVPRGDKTHTCPPCPAPELLGGPSVFLFPPKPKDILMISR |
| | TPEVTCVVVDVSHEDPEVKENWYVDGVEVHNAKTKPREEQYASTYRVVSVLIVLHQDWLNGKEYKCKVSNKALPAPIEKTISKAKG |
| | QPREPQVCTLPPSREEMTKNQVSLSCAVKGFYPSDIAVEWESNGQPEKNYKTTPPVLDSDGSFFLVSKLTVDKSRWQQGNVFSCSVMII |
| | EALANHYTQKSLSLSPGK |
| 67 | EVQLVESGGGLVQPGGSLRLSCAASGFNIKDTYIHWVRQAPGKGLEWVARIYPTNGYTRYADSVKGRFTISADTSKNTAYLQMNSLRA |
| | EDTAVYYCSRWGGDGFYAMDYWGQGTLVTVSSASTKGQVKPEAWISHGPSPGPGHLQLVCHVSGFYPKPVWVMWMRGEQEQQGT |
| | QRGDILPCADGTWYLRATLEVAAGLAADLSCRVKIISSLEGQDIVLYWEGSLVPRGDKTIITCPPCPAPELLGGPSVFLFPPKPKDTLMIS |
| | RTPEVTCVVVDVSHEDPEVKENWYVDGVEVHNAKTKPREFQYASTYRVVSVLTVLHQDWINGKEYKCKVSNKALPAPJEKTISKAKG |
| | QPREPQVCTLPPSREEMTKNQVSLSCAVKGFYPSDIAVEWESNGQPEKNYKTTPPVLDSDGSFFLVSKLTVDKSRWQQGNVFSCSVMH |
| | EALHNHYTQKSLSLSPGK |
| 68 | EVQLVESGGGLVQPGGSLRLSCAASGFNIKDTYIHWVRQAPGKGLEWVARTYPINGYTRYADSVKGRETISADTSKNTAYLQMNSLRA |
| | EDIAVYYCSRWGGDGFYAMDYWGQGTLVTVSSASTKGQVKPEAWLSHGPSPGPGHLQLVCHVSGFYPKPVWVMWMRGEQEQQGT |
| | QRGCILPSADGTWYLRATLEVAAGEAADLSCRVKHSSLEGQDIVLYWEGSLVPRGDKTHTCPPCPAPELLGGPSVELFPPKPKDILMISR |
| | TPEVTCVVVDVSHEDPEVKENWYVDGVEVHNAKIKPREHQYASTYRVVSVLTVLHQDWLNGKEYKCKVSNKALPAPIEKTISKAKG |
| | QPREPQVCILPPSREEMTKNQVSLSCAVKGFYPSDIAYEWESNGQPENNYKTTPPVLDSDGSFFLVSKLTVDKSRWQQGNVESCSVMH |
| | EALHNHYTQKSLSLSPGK |
| 69 | DIQMTQSPSSLSASVGDRVTITCRASQDVNTAVAWYQQKPGKAPKLLIYSASFLYSGVPSRFSGSRSGTDFTLTISSLQPEDEATYYCQQH |
| | YTTPPTFGQGTKVEJKRTIQRTPKICVYSRHPAENGKSNFLNCYVSGFHPSDIEVDLLKNGERIEKVEHSDLSFSKDWSFYLLYYTEFTPT |
| | EKDEYACRVNIIVTLSQPKIVKWDRDM |
| 70 | DIQMTQSPSSLSASVGDRVTITCRASQDVNTAVAWYQQKPGKAPKLLIYSASFLYSGVPSRFSGSRSGTDFTLTISSLQPEDFATYYCQQH |
| | YTTPPTFGQGTKVEIKRTIQRTPKIQVYSRHCAENGKSNFLNCYVSGFHPSDIEVDLLKNGERIEKVEHSDLSESKDWSFYLLYYTEFTPT |
| | EKDEYACRVNHVILSQPKIVKWDRDM |
| 71 | DIQMTQSPSSLSASVGDRVTIICRASQDVNTAVAWYQQKPGKAPKLLIYSASFLYSGVPSRFSGSRSGTDFTLTISSLQPEDFATYYCQQH |
| | YTTPPTEGQGTKVEIKRTIQRTPKIQVYSRHPAENGKSNFENCYVSGFHPSDIEVDLEKNGERIEKVEHSDLSESKQWSFYLLYYTEFTPT |
| | EKDEYACRVNHVTLSQPKIVKWDRDC |
| 72 | EVQLVESGGGLVQPGGSLRLSCAASGFNIKDTYIHWVRQAPGKGLEWVARIYPTNGYTRYADSVKGRFTISADTSKNTAYLQMNSLRA |
| | EDTAVYYCSRWGGDGFYAMDYWGQGTLVTVSSASTKGQVKPEACLSHQPSPGPGHLQLVCHVSGFYPKPVWVMWMRGEQEQQGT |
| | QRGDITPSADGTWYLRATLEVAAGFAADLSCRVKHSSLEGQDIVLYWEGSLVPRGDKTHTCPPCPAPELLGGPSVELFPPKPKDTLMISR |
| | TREVTCVVVDVSHEDPEVKENWYVDGVEVHNAKTKPREEQYASTYRVVSVLTVIHQDWINGKEYKCKVSNKALPAPIEKTISKAKG |
| | QPREPQVCTIPPSREEMTKNQVSLSCAVKGFYPSDIAVEWESNGQPENNYKTTPPVLDSDGSFFLVSKLTVDKSRWQQGKVFSCSVMH |
| | EAULINIIYTQKSLSLSPGK |
| 73 | EVQLVESGGGLVQPGASERLSCAASGENIKQTYIHWYRQAPGKGLEWVARTYPINGYTRYADSVKGRETISADTSKNTAYLQMNSERA |
| | EDTAVYYCSRWGGDGFYAMDYWGQGTLVTVSSASTKGQVKPEAWLSHCPSPGPGHIQLVCHVSGFYPKPVWVMWMRGFQEQQGT |
| | QRGDILPSADGTWYLRATLEVAAGEAADLSCRVKHSSLEGQDIVLYWEGSLVPRGDKTHTCPPCPAPELLGGPSVELFPPKPKDTLMISR |
| | TPEVTCVVVDVSIIEDPEVKENWYVDGVEVIINAKTKPREEQYASTYRVVSVLTVLHIQDWLNGKEYKCKVSNKALPAPIEKTISKAKG |
| | QPREPQVCTLPPSREEMTKNQVSLSCAVKGFYPSDIAVEWESNGQPENNYKTIPPVLDSDGSFFLVSKLTVDKSRWQQGNVFSCSVMH |
| | EALHNHYTQKSLSLSPGK |
| 74 | DIQMTQSPSSLSASVGDRVTITCRASQDVNTAVAWYQQKPGKAPKLLIYSASFLYSGVPSRFSGSRSGTDFTLTISSLQPEDFATYYCQQH |
| | YTTPPTFGQGTKVEIKRTIQRTPKIQVYSRIIPAENGKSNFLNCYVSGFLIPSDIEVDLLKNGERIEKVEHISDLSFSKDWSFYLLYYTEFIPT |
| | EKDEYACRVNHVTLSQPKIVKWDRDMC |
| 75 | DIQMTQSPSSLSASVGDRVTITCRASQDVNTAVAWYQQKPCKAPKLLIYSASFLYSGVPSRFSGSRSGTDFTLTISSLQPEDFATYYCQQH |
| | YTTPPTFGQGTKVEIKRTIQRTPKIKVYSRIIPAENGKSNFLNCYVSGFIIPSDIEVDLLENGERIEKVEIISDLSFSKDWSFYLLYYTEFIPT |
| | EKDEYACRVNIIVTLSQPKIVKWDRDMC |
| 76 | EVQLVRSGGGLVQPGGSLRISCAASGENIKDTYIHWVRQAPGKGLEWVARTYPINGYTRYADSVKGRFTISADTSKNTAYLQMNSERA |
| | EDTAVYYCSRWGGDGFYAMDYWGQGTLVTVSSASTKGQVKPEAWLSHCPSPGPGHLQLVCHVSGFYPKPVWVMWMRGEQEQQGT |
| | QRGDILPSADGTWYLRATLEVAAGEAADLSCRVKIISSLEGQDIVLYWEGSDKTIITCPPCPAPELLGGPSVFLFPPKPKDTLMISRTPEVT |
| | CVVVDVSHEDPEVKENWYVDGVEVHNAKIKPREEQYASTYRVVSVLIVLHQDWLNGKEYKCKVSNKALPAPIEKTISKAKGQPREP |
| | QVCTLPPSREEMTKNQVSLSCAVKGFYPSDIAVEWESNGQPENNYKTTPPVLDSDGSFFLVSKLTVDKSKWQQGNVFSCSVMHEALHN |
| | HYTQKSLSLSPGK |
| 77 | EVQLVESGGGLVQPGGSLRLSCAASGFNIKDTYIHWVRQAPGKGLEWVARTYPINGYTRYADSVKGRFTISADTSKNTAYLQMNSLRA |
| | EDTAVYYCSRWGGDGFYAMDYWGQGTLVTVSSASTKGQVKPEAWLSHCPSPGPGHLQLVCHVSGFYPKPVWVMWMRGEQEQQGT |
| | QRSDILPSADGTWYLRATLEVAAGHAADESERVKHSSLEGQDIVEYWEGSDKTHTCPPCPAPELLGGPSVELFPPKPKQTIMISRTPEVT |
| | CVVVDVSHEDPEVKENWYVDGVEVHNAKTKPREFQYASTYRVVSVLTVLHQDWLNGKEYKCKVSNKALPAPIEKTISKAKGQPREP |
| | QVCTLPPSRHEMTKNQVSLSCAVKGEYPSDIAVEWESNGQPENNYKTTPPVLDSDGSFERSKLTVDKSRWQQGNVESCSVMHEALHN |
| | ITYTQKSLSLSPCK |
| 78 | EVQLVESGGGLVQPGGSLRLSCAASGENIKDTYIHWYRQAPGKGLEWVARIYPINGYTRYADSVKGRFTISADTSKNTAYLQMNSLRA |
| | EDTAVYYCSRWGGDGFYAMDYWGQGTLVTVSSASTKGQVKPEAWLSHCPSPGPGHLQLVCHVSGFYPKPVWVMWMRGEQEQQGT |
| | QRSEILPSADGTWYLRATLEVAAGEAADLSCRVKHSSLEGQDIVLYWEGSDKTHTCPPCPAPELLGGPSVFLFPPKPKDTLMISRTPEVT |
| | CVVVDVSHEDPEVKFNWYVDGVEVIINAKIKPREEQYASTYRVVSVLIVLIIQDWLNGKEYKCKVSNKALPAPIEKTISKAKGQPREP |
| | QVCTIPPSREEMTKNQVSLSCAVKGFYPSDIAVEWESNGQPENNYKTTPPVLDSDGSFFLVSKLTVDKSRWQQGNVFSCSVMHIEALHN |
| | HYTQKSLSLSPGK |
| 79 | EVQLVESGGGLVQPGGSLRLSCAASGFNIKDTYIHWVRQAPGKGLEWVARIYPTNGYTRYADSVKGRFTISADTSKNTAYLQMNSLRA |
| | EDTAVYYCSRWGGDGFYAMDYWQQGTLVTVSSASTKCQVKPEAWLSSCPSPGPGRLQLVCIIVSGFYPKPVWVMWMRGEQEQQGTQ |
| | LGDILPNANWTWYLRATLDVADGEAAGLSCRVKHSSLEGQDIILYWRGSGLNDIFEAQKIEWHEDKTHTCPPCPAPELLGGPSVFLFPP |
| | KPKDTLMISRTPEVTCVVVDVSHEDPEVKFNWYVDGVEVHNAKIKPREEQYASTYRVVSVITVLHQDWINGKEYKCKVSNKALPAPI |
| | EKTISKAKGQPREPQVCTLPPSREEMTKNQVSLSCAVKGFYPSDIAVEWESNGQPENNYKTTPPVLDSDGSFFLVSKLTVDKSRWQQGN |
| | VFSCSVMHFALHNHYTQKSLSLSPGK |
| 80 | EVQLVESGGGLVQPGGSLRLSCAASGFNIKDTYIHWVRQAPGKGLEWVARIYPTNGYTRYADSVKGRFTISADTSKNTAYLQMNSERA |
| | EDTAVYYCSRWGGDGFYAMDYWGQGTLVTVSSASTKGQVKPEAWLSSCPSPGPGRLQLVCHVSGEYPKPVWVMWMRGEQEQQGTQ |
| | LGDILPNANWTWYLRATLDVADGEAAGLSCRYKHSSLEGQDIILYWRGSGDKTHTCPPCPAPELLGGPSVFLFPPKPKDTLMISRTPEVT |
| | CVVVDVSHEDPEVKFNWYVDGVEVHNAKTKPREEQYASTYRVVSVLTVLHQDWLNGKEYKCKVSNKALPAPIEKTISKAKGQPREP |
| | QVCTLPPSREEMTKNQVSLSCAVKGFYPSDIAVEWESNGQPENNYKTTPPVLDSDGSFFLVSKLIVDKSRWQQGNVFSCSVMHEALHN |
| | HYTQKSLSLSPGK |
| 81 | EVQLVESGGGLVQPGGSLRLSCAASGFNIKDTYIHWVRQAPGKGLEWVARIYPTNGYTRYADSVKGRFTISADTSKNTAYLQMNSERA |
| | EDTAVYYCSRWGGDGFYAMDYWGQGTLVTVSSASTKGQVKPEAWLSSCPSPGPGRLQLVCHVSGFYPKPVWVMWMRGEQEQQGTQ |
| | LSDILPNANWTWYLRATLDVADGEAAGLSCRVKHSSLEGQDIILYWRGSGDKTHTCPPCPAPELLGGPSVFLFPPKPKDTLMISRTPEVT |
| | CVVVDVSHEDPEVKFNWYVDGVEVHNAKIKPREEQYASTYRVVSVLTVLHQDWLNGKEYKCKVSNKALPAPIEKTISKAKGQPREP |
| | QVCTIPPSREEMTKNQVSISCAVKGFYPSDIAVEWESNGQPENNYKTTPPVLDSDGSFFLVSKITVDKSRWQQGNVFSCSVMHEALHN |
| | HYTQKSLSLSPGK |
| 82 | EVQLVESGGGLVQPGGSLRLSCAASGENIKDTYIHWVRQAPGKGLEWVARIYPTNGYTRYADSVKGRFTISADTSKNTAYLQMNSLRA |
| | EDTAVYYCSRWGGDGFYAMDYWGQGTLVTVSSAIAELPPKVSVFVPPRDGFFGNPRKSKLICQATGFSPRQIQVSWLREGKQVGSGVT |
| | TDQVQAEAKESGPTTYKVISTLTIKESDWLGQSMFTCRVDHRGLIFQQQASSMCVPDKTHTCPPCPAPELLGGPSVFLFPPKPKDTLMI |
| | SRTPEVTCVVVDVSHEDPEVKFNWYVDGVEVHNAKTKPREEQYASTYRVVSVLTVLHQDWLNGKEYKCKVSNKALPAPIEKTISKAK |
| | GQPREPQVCTLPPSREEMTKNQVSLSCAVKGFYPSDIAVEWESNGQPENNYKTTPPVLDSDGSFELVSKLTVDKSRWQQGNVFSCSVM |
| | HEALHNHYTQKSLSLSPGK |
| 83 | DIQMTQSPSSLSASVQDRVTITCRASQDVNTAVAWYQQKPGKAPKLLIYSASFLYSGVPSRFSGSRSGTDFTLTISSLQPEDFATYYCQQH |
| | YTTPPTFGQGTKVEIKRTVIAELPPKVSVFVPPRDGFFGNPRKSKLICQATGFSPRQIQVSWLREGKQVGSGVTTDQVQAEAKESGPTTY |
| | KVTSTLTIKESDWLGQSMFTCRVDIIRGLTFQQQASSMCVP |
| 84 | EVQLVESGQGIVQPGGSLRLSCAASGENIKDTYIHWVRQAPGKGLEWVARTYPINGYTRYADSVKGRFTISADTSKNTAYLQMNSERA |
| | EDTAVYYCSRWGGDGEYAMDYWGQGTIVTVSSATAELPPKVSVFVPPCDGFFGNPRKSKLYCQATGFSPRQTQVSWEREGKQVGSGV |
| | TTDQVQARAKESGPTTYKVYSTLTIKESDWLGQSMFTCRVDHRGLTFQQQASSMSVPDKTHTCPPCPAPELLGGPSVFLFPPKPKDTLM |
| | ISRTPEVTCVVVDVSHEDPEVKFNWYVDGVEYHNAKTKPREEQYASTYRVVSVLIVLHQDWLNGKEYKCKVSNKALPAPIEKTISKA |
| | KGQPREPQVCTLPPSREEMTKNQVSLSCAVKGEYPSDIAVEWESNGQPENNYKTTPPVLDSDGSEFLVSKLTVDKSRWQQGNVFSCSV |
| | MHEALHNHYTQKSLSLSPGK |
| 85 | DIQMTQSPSSLSASVGDRVTITCRASQDVNTAVAWYQQKPGKAPKLLIYSASFLYSGVPSRFSGSRSGTDFTLTISSLQPEDFATYYCQQH |
| | YTTPPTFGQGTKVEIKRTVIAELPPKVSVFVPPCDGFFGNPRKSKLYCQATGFSPRQIQVSWLREGKQVGSGVTIDQVQAEAKESGPTT |
| | YKVYSTLTIKESDWLGQSMFTCRYDIIRGLTFQQQASSMSVP |
| 86 | EVQLVRSGGGLVQPGGSLRLSCAASGENIKDTYTHWVRQAPGKGLEWVARTYPINGYTRYADSVKGRETISADTSKNTAYLQMNSLRA |
| | EDTAVYYCSRWGGDGFYAMDYWGQGTLVTVSSASTKGQVKPEAWLSHCPSPGPGHLQLVCHVSGFYPKPVWVMWMRGEQFQQGT |
| | QRGDILPSADGTWYLRATLEVAAGEAADLSCRVKHSSLEGQDIVLYWEGSDKTHTCPPCPAPELLGGPSVELFPPKPKDTIMISRTPEVT |
| | CVVVDVSHEDPEVKENWYVDGVEVHNAKTKPREFQYNSTYRVVSVLTVLHQDWINGKEYKCKVSNKALPAPIEKTISKAKGQPREP |
| | QVCTIPPSREEMTKNQVSLSCAVKGFYPSDIAVEWESNGQPENNYKTTPPVLDSDGSFFLVSKITVDKSRWQQGNVFSCSVMHEALHN |
| | HYTQKSESISPQK |
| 87 | EVQLVESGGGLVQPGGSLRESCAASGFNIKDTYIHWVRQAPGKGLEWVARTYPINGYTRYADSVKGRFTISADTSKNTAYLQMNSLRA |
| | EDTAVYYCSRWGGDGFYAMDYWGQGTLVTVSSASTKGQVKPEAWLSIICPSPGPGHILQIVCIIVSGFYPKPVWVMWMRGEQEQQGT |
| | QRSEILPSADGTWYLRATLEVAAGEAADLSCRVKHSSLEGQDIVLYWEGSDKTHTCPPCPAPELLGGPSVFLPPPKPKDTLMISRTPEVT |
| | CVVVDVSHEDPEVKFNWYVDGVEVHNAKTKPREEQYNSTYRVVSVLTVLHQDWLNGKEYKCKVSNKALPAPIEKTISKAKGQPREP |
| | QVCTLPPSREEMTKNQVSLSCAVKGFYPSDIAVEWESNGQPENNYKTTPPVLDSDGSFFLVSKLTVDKSRWQQGNVESCSVMHEALHN |
| | HYTQKSLSLSPGK |
| 88 | EVQLVESGGGLVQPGGSLRLSCAASGFNIKDTYIHWVRQAPGKGLEWVARIYPTNGYTRYADSVKGRFTISADTSKNTAYLQMNSLRA |
| | EDTAVYYCSRWGGDGFYAMDYWGQGTLVTVSSASTKGQVKPEAWLSSCPSPGPGRLQLVCHVSGFYPKPVWVMWMRGEQEQQGTQ |
| | LGDILPNANWTWYLRATLDVADGEAAGLSCRVKHSSLEGQDIILYWRGSGDKTHTCPPCPAPELLGGPSVELFPPKPKDTLMISRTPEVT |
| | CVVVDVSHEDPEVKFNWYVDGVEVHNAKTKPREEQYNSTYRVVSVLTVLHQDWLNGKEYKCKVSNKALPAPIEKTISKAKGQPREP |
| | QVCTLPPSREIMTKNQVSLSCAVKGFYPSDIAVEWESNGQPENNYKTTPPVLDSDGSFFLVSKLIVDKSRWQQGNVESCSVMHEALHN |
| | HYTQKSLSLSPGK |
| 89 | EVQLVESGGGLVQPGGSLRLSCAASGFNIKDTYIHWVRQAPGKGLEWVARTYPINGYTRYADSVKGRFTISADISKNIAYLQMNSIRA |
| | EDIAVYYCSRWGGDGFYAMDYWGQGTLVTVSSASTKGQVKPEAWLSSCPSPGPGRLQLVCHVSGFYPKPVWVMWMRGEQEQQGTQ |
| | LSEILPNANWTWYLRATLDVADGEAAGLSCRVKHSSLEGQDIILYWRGSGDKTHTCPPCPAPELLGGPSVFLFPPKPKDTLMISRTPEVT |
| | CVVVDVSHEDPEVKENWYVDGVEVHNAKTKPREEQYNSTYRVVSVLIVLHQDWLNGKEYKCKVSNKALPAPIEKTISKAKGQPREP |
| | QVCTIPPSREEMTKNQVSLSCAVKGFYPSDIAVEWESNGQPENNYKTTPPVLDSDGSFFLVSKILTVDKSRWQQGNVFSCSVMHRALHN |
| | HYTQKSLSLSPGK |
| 101 | EVQLVESGGGLVQPGGSLRLSCAASGFPFNTEAMNWVRQAPGKGLEWLARIRSKSNNYATYYADSVKDRFTISRDNAKRSLYLQMNSL |
| | RAEDTAVYYCVRGKENNYGYAMDYWGQGTTYTVSSASTKGPSVFPLAPSSKSTSGGTAALCLVKDYFPEPVTVSWNSGALTSGVHT |
| | FPAVLQSSGLYSLSSVVTVPSSSLGTQTYICNVNHKPSNTKVDKKVEPKSCDKTHTCPPCPAPELLGGPDVFLFPPKPKDTLMISRTPEVT |
| | CVVVDVSIIEDPEVKENWYVDGVEVIINAKIKPREEQYKSTYRVVSVLIVLIIQDWLNQKEYKCKVSNKALPAPEEKTISKAKGQPREP |
| | QVYTLPPSREEMTKNQVSLTCLVKGFYPSDIAVEWESNGQPENNYKTTPPVLDSDGSFFLYSKLTVDKSRWQQGNVFSCSVMHEALHN |
| | HYTQKSLSLSPGK |
| 102 | DIVMTQSPLSLPVTPGEPASISCRSSKSLLHSNANTYLYWFLQKPGQSPQLLIYRMSNLASGVPDRESGSGSGTDFTLKISRVEAEDVGVY |
| | YCMQIVEYPFTFGGGTKLEIKRTVAAPSVFIFPPSDEQLKSGTASVVCLLNNFYPREAKVQWKVDNALQSGNSQESVTEQDSKDSTYS |
| | LASTLTLSKADYEKHKVYACEVTHQGLSSPVTKSENRGEC |
| 103 | QVQLQESGPGLMKPSETLSLTCSVSGDSIRSDYWSWIRQPPGKGLEYIGYVSYSGSTYYNPSLKSRVTISVDISKNRFSLKLNSVTAADT |
| | AVYYCARWDGDYWGQGILVTVSSASTKGPSVFPLAPSSKSTSGGTAALGCLVKDYFPEPVTVSWNSGALTSGVHTFPAVLQSSGLYSLS |
| | SVVTVPSSSLGTQTYICNVNHKPSNTKVDKKVEPKSCDKTHTCPPCPAPELLGGPSVFLEPPKPKDTLMISRTPEVICVVVDVSHEDPEV |
| | KFNWYVDGVRVHNAKTKPREEQYASTYRVVSVITVIHQDWINGKEYKCKVSNKALPAPIEKTISKAKGQPREPQVYTIPPSREEMTK |
| | NQVSLTCLVKGFYPSDIAVEWESNGQPENNYKTIPPVLDSDGSFFLYSKLTVDKSRWQQGNVESCSVMHEALHNHYTQKSLSLSPGK |
| 104 | EVQLVESGGGEVQPGGSLRISCAANGYSFTGYTMNWVRQAPGKGLEWVALINPYKGVSTYNQKFKDRETISVDKSKKTAYLQMNSLR |
| | AEDIAVYYCARSGYYGDSDWYFDVWGQGTLVTVSSASTKGPSVPPLAPSSKSTSGGTAALGCLVKDYFPEPVTVSWNSGALTSGVHT |
| | FPAVLQSSGLYSLSSVVTVPSSSLGTQTYICNVNIKPSNTKVDKKVEPKSCDKTIITCPPCPAPELLGGPSVELFPPKPKDTLMISRTPEVT |
| | CVVVDVSHEDPEVKFNWYVDGVEVHNAKTKPREEQYASTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPVLDSDGSFFLYSKLTYDKSRWQQGNVESCSVMHEALHN |
| | HYTQKSLSLSPGK |
| 105 | DIVMTQSPLSLPVTPGEPASISCRSSKSLLISNANTYLYWFLQKPGQSPQLLIYRMSNLASGVPDRESGSGSGTDFILKISRVEAEDVGVY |
| | YCMQHVEYPFTFGGGTKLEIKRTIQRTPKIKVYSRHPAENGKSNFLNCYVSGFHPSDIEVDLLKNGERIEKVEHSDLSFSKDWSFYLLYY |
| | TEFTPTEKDEYAGRVNHVTLSQPKIVKWDRQMC |
| 106 | EVQLVESGGGLVQPGGSLRISCAASGFPFNTFAMNWVRQAPGKGLEWLARIRSKSNNYATYYADSVKDRETISRDNAKRSLYLQMNSL |
| | RAEDTAVYYCVRGKENNYGYAMDYWGQGTTVTVSSASTKGQVKPEAWLSHCPSPGPGHLQLVCHVSGFYPKPVWVMWMRGEQEQ |
| | QGTQRSEILPSADGTWYLRATLEVAAGEAADLSCRVKHSSLEGQDIVLYWEGSDKTHTCPPCPAPELLGGPSVELFPPKPKDTIMISRTP |
| | EVTCVVVDVSHEDPEVKFNWYVDGVEVIINAKTKPRECQYASTYRVVSVLTVLIIQDWLNGKEYKCKVSNKALPAPIEKTISKAKGQP |
| | REPQVCTLPPSREEMIKNQVSLSCAVKGFYPSDLAVEWESNGQPENNYKTTPPVLDSDGSFFLVSKLTVDKSRWQQGNVFSCSVMHEA |
| | LHNHYTQKSLSLSPGK |
| 107 | EVQLVESGGGLVQPGGSLRLSCAASGEPFNTFAMNWVRQAPGKGLEWLARIRSKSNNYATYYADSVKDRFTISRDNAKRSLYLQMNSL |
| | RAEDTAVYYCVRGKENNYGYAMDYWGQGTTVTVSSASTKGQVKPEAWLSSCPSPGPGRLQLVCHVSGFYPKPVWVMWMRGEQEQ |
| | QGTQLSEILPNANWTWYLRATLDVADGEAAGLSCRVKHSSLEGQDIILYWRGSGDKTHTCPPCPAPELLGGPSVFLFPPKPKDILMISRT |
| | PEVTCVVVDVSHEDPEVKFNWYVDGVEVHNAKTKPREEQYASTYRVVSVLTVLHQDWINGKEYKCKVSNKALPAPIEKTISKAKGQP |
| | REPQVCTIPPSREEMTKNQVSLSCAVKGFYPSDIAVEWESNGQPENNYKTTPPVLDSDGSFFLVSKLTVDKSRWQQGNVESCSVMHEA |
| | LHNITYTQKSLSLSPGK |
| 108 | EVQLVESGGGLVQPQGSERISCAASGENIKDTYIHWVRQAPGKGLEWVARTYPINGYTRYADSVKGRETISADTSKNTAYLQMNSERA |
| | EDTAVYYCSRWGGDGFYAMDYWGQGTLVTVSSASTKGQVKPEAWLSIICPSPGPGIILQLVCLIVSGFYPKPVWVMWMRGEQEQQGT |
| | QRGDILPSADQTWYLRATLEVAAGLAADLSCRVKIISSLEGQDIVLYWEGSHHHHHHHH |
| 109 | EVQLVESGGGLVQPGGSLRLSCAASGENIKDTYIHWVRQAPGKGLEWVARIYPTNGYTRYADSVKGRFTISADTSKNTAYLQMNSLRA |
| | EDTAVYYCSRWGGDGFYAMDYWGQGTLVTVSSASTKCQVKPEAWLSIICPSPGPGIILQLVCHIVSGFYPKPVWVMWMRGEQEQQGT |
| | QRSDILPSADGTWYLRAILEVAAGEAADLSCRVKHSSLEGQDIVLYWEGSHHHHHHHH |
| 110 | EVQLVESGGGLVQPGGSLRLSCAASGENJKDTYIHWVRQAPGKGLEWVARIYPTNGYTRYADSVKGRFTISADTSKNTAYLQMNSERA |
| | EDTAVYYCSRWGGDGFYAMDYWGQGTLVTVSSASTKGQVKPEAWLSIICPSPGPGIILQLVCIIVSGFYPKPVWVMWMRGEQEQQGT |
| | QRSEILPSADGTWYLRATLEVAAGEAADLSCRVKHSSLEGQDIVLYWEGSHHHHHHHH |
| 111 | LVQLVESGQGLVQPGGSLRLSCAASGENIKDTYIIIWVRQAPCKGLEWVARIYPTNGYTRYADSVKGRFTISADTSKNTAYLQMNSLRA |
| | EDTAVYYCSRWGGDGFYAMDYWGQGTLVTVSSASTKGPSVFPLAPSSKSTSGGTAALGCLVKDYFPEPVIVSWNSGALTSGVHTFPAV |
| | LQSSGLYSLSSVYTVPSSSLGTQTYICNVNHKPSNTKVDKKVEPKSCHHHHHHHH |

### Example 1: Design and Engineering of Antibodies with Various Human Homo/Heterodimerization Domains

### 1. Materials, Method and Strategies

### 1) Antibodies

Antibody A is anti-HER2 antibody Trastuzumab developed by Roche. Antibody A provides light chain 1 and heavy chain 1.

Antibody B is an anti-FAP antibody clone 3F2 developed by Roche, and its sequence is from patent No. WO 2012/020006. Antibody B provides light chain 2 and heavy chain 2.

### 2) Bispecific Antibody (BsAb)

All BsAbs used here comprise four chains: the heavy chain 1, the light chain 1, the heavy chain 2 and the light chain 2. The heavy chain 1 and the light chain 1 can form half-body A, the heavy chain 2 and the light chain 2 can form half-body B.

In order to solve the problem of the heavy chain mismatching, we introduced knob (T336W)-into-hole (T336S, L368A, Y407V) to CH3 domains of the BsAbs and added a stabilizing disulfide bridge in CH3 (S354C in heavy chain 1 and Y349C in heavy chain 2). Specifically, heavy chain 1 of Trastuzumab was cloned into the hole chain with N297A mutation (SEQ ID NO: 6), while heavy chain 2 of anti-FAP antibody was cloned into the knob chain (SEQ ID NO: 2). For better evaluating the assembly of heavy chain and light chain, the C-terminus of anti-FAP light chain 2 was fused with a SUMO tag (SEQ ID NO: 4).

The combinations of the heavy chains and the light chains of BsAbs are summarized in Table 1. The sequences of homo/heterodimerization domains used to replace CH1 or CL and the tag sequence are summarized in Table 2.

TABLE 1

| | | H chain 1 | L Chain1 | H chain2 | L Chain2 | Molar ratio of |
|---|---|---|---|---|---|---|
| No. | Name | (HC1) | (LC1) | (HC2) | (LC2) | HC1:LC1:HC2:LC2 |
| 1 | ABC055 | AP082 | AP081 | AP004 | AP080 | 1:1:1:1 |
| | CH3^{IgG}/CH3^{IgG} | SEQ ID NO: 6 | SEQ ID NO: 5 | SEQ ID NO: 2 | SEQ ID NO: 4 | |
| | | H chain of anti-HER2 | L chain of anti-HER2 | H chain of anti-FAP | L chain of anti- | |
| | | Trastuzumab with hole | Trastuzumab | antibody with knob | FAP antibody | |
| | | mutations CH3 | | mutations on CH3 | with a C- | |
| | | domain and N297A on | | domain | terminal SUMO | |
| | | CH2. | | | tag. | |
| 2 | ABC056 | AP083 | AP085 | AP004 | AP080 | 1:1:1:1 |
| | (knob^{IgGCH3}/ | SEQ ID NO: 7 | SEQ ID NO: 9 | SEQ ID NO: 2 | SEQ ID NO: 4 | |
| | hole^{IgGCH3}) | H chain of Trastuzumab | L chain of Trastuzumab | | | |
| | | with CH1 replaced by | with CL replaced by hole | | | |
| | | knob CH3 domain. | CH3 domain. | | | |
| 3 | ABC057 | AP084 | AP086 | AP004 | AP080 | 1:1:1:1 |
| | (hole^{IgGCH3}/ | SEQ ID NO: 8 | SEQ ID NO: 10 | SEQ ID NO: 2 | SEQ ID NO: 4 | |
| | knob^{IgGCH3}) | H chain of Trastuzumab | L chain of Trastuzumab | | | |
| | | with CH1 replaced by | with CL replaced by knob | | | |
| | | hole CH3 domain. | CH3 domain. | | | |
| 4 | ABC058 | AP091 | AP095 | AP004 | AP080 | 1:1:1:1 |
| | (IL2Rβ/γ) | SEQ ID NO: 15 | SEQ ID NO: 16 | SEQ ID NO: 2 | SEQ ID NO: 4 | |
| | | H chain of Trastuzumab | L chain of Trastuzumab | | | |
| | | with CH1 replaced by | with CL replaced byγ | | | |
| | | IL2Rβ. | subunit. | | | |
| 5 | ABC060 | AP089 | AP087 | AP004 | AP080 | 1:1:1:1 |
| | (HLA/β2m) | SEQ ID NO: 13 | SEQ ID NO: 11 | SEQ ID NO: 2 | SEQ ID NO: 4 | |
| | | H chain of Trastuzumab | L chain of Trastuzumab | | | |
| | | with CH1 replaced by | with CL replaced by β2m. | | | |
| | | HLA α3 domain. | | | | |
| 6 | ABC061 | AP090 | AP088 | AP004 | AP080 | 1:1:1:1 |
| | (β2m/HLA) | SEQ ID NO: 14 | SEQ ID NO: 12 | SEQ ID NO: 2 | SEQ ID: NO: 4 | |
| | | H chain of Trastuzumab | L chain of Trastuzumab | | | |
| | | with CH1 replaced by | with CL replaced by HLA | | | |
| | | β2m. | α3 domain. | | | |
| 7 | ABC064 | AP102 | AP103 | AP004 | AP080 | 1:1:1:1 |
| | (Fos/Jun) | SEQ ID NO: 17 | SEQ ID NO: 18 | SEQ ID NO: 2 | SEQ ID NO: 4 | |
| | | H chain of Trastuzumab | L chain of Trastuzumab | | | |
| | | with CH1 replaced by: | with CL replaced by Jun. | | | |
| | | Fos | | | | |
| 8 | ABC074 | AP177 | AP176 | AP004 | AP080 | 1:1:1:1 |
| | (GABA1/ | SEQ ID NO: 22 | SEQ ID NO: 21 | SEQ ID NO: 2 | SEQ ID NO: 4 | |
| | GABA2) | H chain of Trastuzumab | L chain of Trastuzumab | | | |
| | | with CH1 replaced by | with CL replaced by | | | |
| | | GABA1. | GABA2. | | | |
| 9 | ABC075 | AP179 | AP178 | AP004 | AP080 | 1:1:1:1 |
| | (GABA2/ | SEQ ID NO: 24 | SEQ ID NO: 23 | SEQ ID NO: 2 | SEQ ID NO: 4 | |
| | GABA1) | H chain of Trastuzumab | L chain of Trastuzumab | | | |
| | | with CH1 replaced by | with CL replaced by | | | |
| | | GABA2. | GABA1. | | | |
| 10 | ABC131 | AP172 | AP173 | AP004 | AP080 | 1:1:1:1 |
| | (SPIRα/ | SEQ ID NO: 19 | SEQ ID NO: 20 | SEQ ID NO: 2 | SEQ ID NO: 4 | |
| | CD47) | H chain of Trastuzumab | L chain of Trastuzumab | | | |
| | | with CH1 replaced by | with CL replaced by CD47. | | | |
| | | SPIRα. | | | | |
| 11 | ABC132 | AP090 | AP182 | AP004 | AP080 | 1:1:1:1 |
| | (β2m/CD1b) | SEQ ID NO: 14 | SEQ ID NO: 26 | SEQ ID NO: 2 | SEQ ID NO: 4 | |
| | | H chain of Trastuzumab | L chain of Trastuzumab | | | |
| | | with CH1 replaced by | with CL replaced by CD1b | | | |
| | | β2m. | α3 domain. | | | |
| 12 | ABC133 | AP183 | AP087 | AP004 | AP080 | 1:1:1:1 |
| | (CD1a/β2m) | SEQ ID NO: 27 | SEQ ID NO: 11 | SEQ ID NO: 2 | SEQ ID NO: 4 | |
| | | H chain of Trastuzumab | L chain of Trastuzumab | | | |
| | | with CH1 replaced by | with CL replaced by β2m. | | | |
| | | CD1a α3 domain. | | | | |
| 13 | ABC134 | AP190 | AP189 | AP004 | AP080 | 1:1:1:1 |
| | (CD8a/ | SEQ ID NO: 30 | SEQ ID NO: 29 | SEQ ID NO: 2 | SEQ ID NO: 4 | |
| | CD8β) | H chain of Trastuzumab | L chain of Trastuzumab | | | |
| | | with CH1 replaced by | with CL replaced by CD8β | | | |
| | | CD8a. | | | | |
| 14 | ABC570 | AP590 | AP591 | AP008 | AP080 | 1:1:1:1 |
| | (CH2^{IgM}/ | SEQ ID NO: 82 | SEQ ID NO: 83 | SEQ ID NO: 3 | SEQ ID NO: 4 | |
| | CH2^{IgM}) | H chain of anti-HER2 | L chain of anti-HER2 | H chain of anti-FAP | L chain of anti- | |
| | | Trastuzumab with CH1 is | Trastuzumab with CL is | antibody with knob | FAP antibody | |
| | | replaced by IgM WT | replaced by IgM WT CH2 | mutations on CH3 | with a C-terminal | |
| | | CH2 domain. | domain | domain | SUMO tag. | |
| 15 | ABC571 | AP594 | AP591 | AP008 | AP080 | 1:1:1:1 |
| | knob^{IgMCH2}/ | SEQ ID NO: 84 | SEQ ID NO: 83 | SEQ ID NO: 3 | SEQ ID NO: 4 | |
| | hole^{IgMCH2} | H chain of anti-HER2 | L chain of anti-HER2. | H chain of anti-FAP | L chain of anti- | |
| | | Trastuzumab with CH1 is | Trastuzumab with CL is | antibody with knob | FAP antibody | |
| | | replaced by IgM CH2 | replaced by IgM WT CH2 | mutations on CH3 | with a C-terminal | |
| | | domain containing knob | domain. | domain | SUMO tag. | |
| | | mutations | | | | |
| 16 | ABC572 | AP590 | AP595 | AP008 | AP080 | 1:1:1:1 |
| | hole^{IgMCH2}/ | SEQ ID NO: 82 | SEQ ID NO: 85 | SEQ ID NO: 3 | SEQ ID NO: 4 | |
| | knob^{IgMCH2} | H chain of anti-HER2 | L chain of anti-HER2 | H chain of anti-FAP | L chain of anti- | |
| | | Trastuzumab with CH1 is | Trastuzumab with CL is | antibody with knob | FAP antibody | |
| | | replaced by IgM WT | replaced by IgM CH2 | mutations on CH3 | with a C-terminal | |
| | | CH2 domain. | domain containing knob | domain and N297A | SUMO tag. | |
| | | | mutations. | mutation. | | |

TABLE 2
The important sequences which are used to replace CH1 or CL and the tag
sequence.

| | SEQ | |
|---|---|---|
| Name | ID NO | Amino Acid or DNA Sequence: |
| HLA (A*02:01) | 48 | RTQAPKTHMTHHAVSDHEATLRCWALSFYPAEITLTWQRDGEDQTQDTELVETRPAGDGTF |
| | | QKWAAVVVPSGQEQRYTCHVQHEGLPKPLTLRWEPLSS |
| β2m (1-99) | 49 | IQRTPKIQVYSRHPAENGKSNFLNCYVSGFHPSDIEVDLLKNGERIEKVEHSDLSFSKDWSF |
| | | YLLYYTEFTPTEKDEYACRVNIIVTLSQPKIVKWDRDM |
| CD1a (184-278) | 50 | QVKPEAWLSHGPSPGPGHLQLVCHVSGFYPKPVWVMWMRGEQEQQGTQRGDILPSADGT |
| | | WYLRATLEVAAGEAADLSCRVKHSSLEGQDIVLYWEGSLVPRG |
| CD1b (184-278) | 51 | QVKPEAWLSSGPSPGPGRLQLVCHVSGFYPKPVWVMWMRGEQEQQGTQLGDILPNANWT |
| | | WYLRATLDVADGEAAGLSCRVKHSSLEGQDIILYWRGSGLNDIFEAQKIEWHE |
| GABA1 (878-919) | 52 | STNNNEEEKSRLLEKENRELEKIIAEKEERVSELRHQLQSR |
| GABA2 (779-819) | 53 | SVNQASTSRLEGLQSENHHLRMKITELDKDLEEVTMQLQDT |
| with R797H | | |
| mutation | | |
| Jun | 54 | RRELTDTLQAETDQLEDEKSALQTEIANLLKEKEKLEFILA |
| Fos | 55 | KLERIARLEEKVKTLKAQNSELASTANMLREQVAQLKQKVM |
| SUMO tag | 56 | MSDSEVNQEAKPEVKPEVKPETHINLKVSDGSSEIFFKIKKTTPLRRLMEAFAKRQGKEMD |
| | | SLRFLYDGIRIQADQTPEDLDMEDNDIIEAIIREQIGG |
| IgM CH2 WT domain | 90 | IAELPPKVSVFVPPRDGFFGNPRKSKLICQATGFSPRQIQVSWLREGKQVGSGVTTDQVQAE |
| with N332Q | | AKESGPTTYKVTSTLTIKESDWLGQSMFTCRVDIIRGLTFQQQASSMCVP |
| IgM CH2 WT domain | 91 | ATCGCCGAGCTGCCCCCCAAGGTGAGGGTGTTCGTGCCCCCTAGAGACGGCTTCTTCGG |
| with N332Q | | CAACCCTAGAAAGAGCAAGCTGATCTGCCAAGCCACCGGCTTCAGCCCTAGACAGATC |
| | | CAAGTGAGCTGGCTGAGAGAGGGCAAGCAAGTGGGCAGCGGCGTGACAACCGACCAA |
| | | GTGCAAGCCGAGGCCAAGGAGAGCGGCCCCACCACCTACAAGGTGACAAGCACCCTG |
| | | ACCATCAAGGAGAGCGACTGGCTGGGGCAGAGCATGTTCACCTGCAGAGTGGACCACA |
| | | GAGGCCTGACCTTTCAGCAACAAGCTAGCAGCATGTGCGTGCCC |
| HLA (A*02:01) | 92 | AGAACAGACGCCCCTAAGACACACATGACCCACCACGCCGTGTCCGATCACGAAGCCA |
| | | CACTGAGATGTTGGGCCCTGAGCTTCTACCCCGCCGAGATCACACTGACCTGGCAGAGA |
| | | GATGGCGAGGATCAGACCCAGGATACCGAGCTGGTGGAAACAAGACCTGCCGGCGACG |
| | | GCACCTTCCAGAAATGGGCTGCTGTGGTGGTGCCTAGCGGCCAAGAGCAGAGATACAC |
| | | CTGTCACGTGCAGCAGAGGCCTGCCTAAGCCTCTGACTCTGAGATGGGAGCTGAGC |
| | | AGC |
| β2m (1-99) | 93 | ATCCAGCGGACCCCTAAGATCCAGGTGTACAGCAGACACCCCGCCGAGAACGGCAAGA |
| | | GCAACTTCCTGAACTGCTACGTGTCCGGCTTTCACCCCAGCGACATTGAGGTGGACCTG |
| | | CTGAAGAACGGCGAGCGGATCGAGAAGGTGGAACACAGOGATCTGAGCTTCAGCAAG |
| | | GACTGGTCCTTCTACCTGCTGTACTACACCGAGTTCACCCCTACCGAGAAGGACGAGTA |
| | | CGCCTGCAGAGTGAACCACGTGACACTGAGCCAGCCTAAGATCGTGAAGTGGGACAGA |
| | | GACATG |
| CD1a (184-278) | 94 | CAAGTGAAACCTGAGGCCTGGCTTTCCCATGGTCCAAGCCCTGGCCCTGGACATCTGCA |
| | | GCTTGTCTGTCATGTCAGTGGGTTCTACCCTAAGCCAGTGTGGGTGATGTGGATGAGGG |
| | | GTGAGCAGGAGCAGCAGGGCACCCAACGAGGAGACATCCTGCCTTCTGCTGATGGCAC |
| | | CTGGTACCTTCGGGCTACCTTGGAAGTGGCAGCCGGAGAGGCCGCCGATCTTAGTTGTC |
| | | GGGTCAAGCATTCATCCCTGGAGGGGCAAGACATCGTGTTGTACTGGGAAGGCTCTCTG |
| | | GTTCCCAGAGGG |
| CD1b ((184-278) | 95 | CAGGTGAAGCCTGAGGCTTGGCTGAGTTCCGGACCAAGCCCCGGTCCGGGTCGGCTGC |
| | | AGCTGGTGTGCCACGTGTCAGGATTCTATCCCAAGCCCGTATGGGTCATGTGGATGAGA |
| | | GGAGAACAAGAGCAGCAAGGGACCCAGCTCGGCGACATCCTGCCCAATGCAAATTGGA |
| | | CATGGTACCTGCGCGCAACTCTGGATGTGGCGGATGGAGAGGCCGCCGGACTGTCTTGC |
| | | CGAGTGAAACATTCTTCACTGGAGGGCCAGGATATCATCCTCTACTGGCGGGGTTCCGG |
| | | CCTGAATGACATCTTCGAGGCCCAGAAAATTGAGTGGCATGAG |
| GABA1 (878-919) | 96 | AGTACAAATAACAACGAGGAGGAGAAGTCTCGGCTTCTCGAGAAGGAAAACCGGGAA |
| | | CTGGAGAAGATTATTGCAGAGAAAGAGGAGCGCGTTTCTGAGCTGCGCCACCAGCTGC |
| | | AGAGCAGA |
| GABA2 (779-819) | 97 | TCTGTGAATCAGGCAAGCACTAGCAGACTTGAAGGACTCCAGTCCGAGAATCACCACT |
| with R797H | | TGCGAATGAAGATCACCGAGTTGGATAAGGACCTGGAGGAAGTGACAATGCAGCTGCA |
| mutation | | GGATACC |
| Jun | 98 | AAGCTGGAACGGATCGCCCGGCTGGAAGAGAAAGTGAAAACCCTGAAGGCCCAGAAC |
| | | AGCGAGCTGGCCTCTACCGCCAACATGGTGAGAGAACAGGTGGCCCAGCTGAAGCAGA |
| | | AAGTCATG |
| Fos | 99 | CGGAGAGAGCTGACCGATACACTGCAGGCCGAGACAGACCAGCTGGAAGATGAGAAG |
| | | TCTGCCCTGCAGACCGAGATCGCCAACCTGCTGAAAGAGAAAGAGAAGCTCGAGTTCA |
| | | TCCTGGCC |
| SUMO tag | 100 | ATGAGCGACAGCGAAGTGAATCAAGAGGCCAAGCCTGAAGTGAAACCCGAAGTGAAG |
| | | CCTGAGACACACATCAACCTGAAGGTGTCCGACGGCAGCAGCGAGATCTTCTTCAAGA |
| | | TCAAGAAAACCACACCTCTGCGGCGGCTGATGGAAGCCTTCGCCAAAAGACAGGGCA |
| | | AAGAGATGGACAGCCTGCGGTTCCTGTACGACGGCATCAGAATCCAGGCCGATCAGAC |
| | | CCCTGAGGACCTGGACATGGAAGATAACGACATCATCGAGGCCCACAGAGAGCAGATC |
| | | GGAGGA |

### 3) Method: Expression and Purification

The desired DNA sequences of heavy chains and light chains were inserted into the mammalian expression vector pCI (Promega #E1731) between NheI and Xmal. BsAbs were produced by co-transfection of 4 plasmids (HC1, LC1, HC2, LC2) into Expi293F cells (ThermoFisher #A14527) following the manufacturer's recommendations. Expi293F cells were grown in suspension culture. The supernatant was harvested six days post-transfection by centrifugation and further filtered with 0.22 m filter. Antibodies were purified with protein A beads from the filtered supernatant. Antibody concentration was measured by Nanodrop (NanoDrop2000, Thermo scientific) at 280 nm.

### 4) Evaluation of the Light Chain-Heavy Chain Pairing: The Cognate LC/HC Pairing was Evaluated by SDS-PAGE.

### Method: SDS-PAGE Analysis of BsAbs

After protein A purification, the assembly of BsAb was analyzed by SDS-PAGE. The samples mixed with loading buffer were heated at 95° C. for 5 min with/without DTT and electrophoresed on 4-20% gels in MOPS running buffer (Bio-Rad). The gels were then stained with Coomassie blue (Thermo Scientific) and destained in water. An equal amount of protein (6 μg) was loaded for each sample. For better distinguishing of two heavy chains, 20 μg BsAbs (ABC056, ABC057, ABC060, ABC061, ABC131, ABC132 and ABC133) were deglycosylated by 2 μl PNGase F 500U/μl (NEB #P0704L) at 37° C. for 1 hour.

### Results:

BsAbs were produced by co-expressing two different heavy chains (HCs) and two different light chains (LCs) in a single host cell line, by substituting CH1/CL of the half-body A with other human homo/heterodimerization domains or proteins, including non-IgG-like and IgG-like heterodimers. The non-IgG-like heterodimers used in the design include Fos/Jun, GABA1/GABA2 and GABA2/GABA1. The IgG-like domains used in the bispecific assembly include interleukin receptor pairs (IL2Rβ/γ), subunits pairs of CD3, CD8a/CD8β, CH3/CH3 of IgG1 (Knobs-into-holes), CH2/CH2 of IgM (WT and mutations), HLA-α3/β2m, β2m/HLA-α3, SIPRα/CD47, β2m/CD1s-α3 and CD1s-α3/β2m. For the initial screening, all engineered BsAbs were expressed with a HC1:LC1:HC2:LC2 molar ratio at 1:1:1:1. The resultant hetero-IgG1 mixture containing BsAb was purified by protein A beads and then analyzed by SDS-PAGE gel to evaluate the cognate LC/HC pairing.

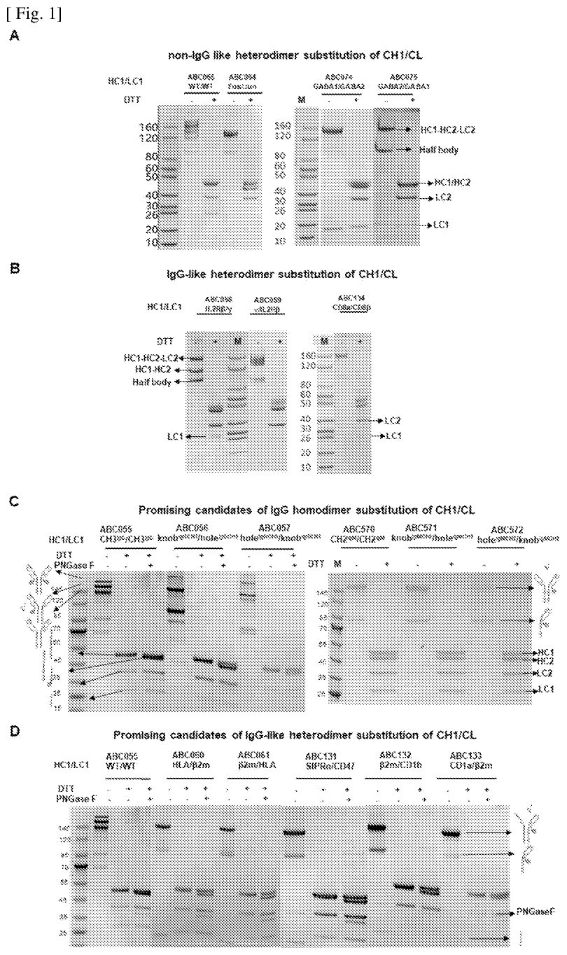

For the parental anti-HER2/anti-FAP BsAb which has no engineering in the CH1/CL domains of half-body A, three bands around 160 kDa were observed (FIG. 1A, 1C, ABC055). The upper and the lower bands represent one LC miss-pairing species, the middle band corresponds to the correct BsAb configuration and/or the LC-swapped species. Therefore, less than 30% correctly-assembled BsAb was observed in the mixture of IgG1 species.

Since there is no interchain disulfide bond within the engineered CH1/CL domain of half-body A, the engineered LC1 should be separated from the BsAb on a non-reducing SDS-PAGE gel. Thus, a successfully assembled BsAb will be revealed with two bands in the non-reducing condition: a band corresponding to the configuration of HC1/HC2/LC2-SUMO and a band corresponding to the unlinked LC1. In addition, four bands are expected to be detected under the reducing condition, although two HCs might be too close to distinguish. Based on these criteria, Fos/Jun (ABC064) pair substitution failed as the engineered LC1 was not detected on the non-reducing condition (FIG. 1A), indicating the engineered LC1 did not assemble well with the HC1/HC2/LC2-SUMO. While GABA1/GABA2 pair substitution (ABC074 and ABC075) showed correct bands (FIG. 1A). For the IgG-like domain substitution (FIG. 1B), replacement of CH1/CL with IL2Rβ/γ (ABC058) and CD8a/CD8β (ABC134) domain likely led to not promising assembly as LC1 was not observed under non-reducing (FIG. 1B).

As shown in FIG. 1C, the CH3/CH3 knobs-into-holes domain with the interchain disulfide bond between CH3/CH3 domain (ABC056 and ABC057) led to better BsAb assembly compared to WT (FIG. 1C). Furthermore, cognate HC/LC pairing was achieved when wild-type IgM CH2 domains or IgM CH2 bearing knobs-in-holes replaced the CH1/CL domain (ABC570, ABC571, ABC572). Similarly, IgE CH2/CH2 domain replacement is expected to achieve cognate chain pairing as IgM CH2/CH2 domain replacement.

As shown in FIG. 1D, the promising HC/LC pairing was observed when the CH1/CL domain of the HC1 and LC1 was replaced with HLA-α3/β2m or β2m/HLA-α3 (ABC060 and ABC061), SIRPα/CD47 (ABC131), β2m/CD1b (ABC132), CD1a/β2m (ABC133), because two expected bands were observed under non-reducing conditions, and all four chains were detected on reducing condition after PNGase F treatment. ABC133 (CD1a/β2m) was the most promising design that resulted in high purity of bispecific product with the least amount of half bodies. IgG-like heterodimer substitution of CH1/CL that showed correct HC/LC pairing is summarized in Table 3.

TABLE 3
IgG-like homo/heterodimer substitution of CH1/CL that showed correct HC/LC pairing

| | | H chain 1 | L Chain1 | H chain2 | L Chain2 | Molar ratio of |
|---|---|---|---|---|---|---|
| No. | Name | (HC1) | (LC1) | (HC2) | (LC2) | HC1:LC1:HC2:LC2 |
| 1 | ABC056 | AP083 | AP085 | AP004 | AP080 | 1:1:1:1 |
| | (knob/hole) | SEQ ID NO: 7 | SEQ ID NO: 9 | SEQ ID NO: 2 | SEQ ID NO: 4 | |
| | | H chain of | L chain of | H chain of anti- | L chain of | |
| | | Trastuzumab with | Trastuzumab with | FAP antibody | anti-FAP | |
| | | CH1 replaced by | CL replaced by hole | with knob | antibody with | |
| | | knob CH3 domain. | CH3 domain. | mutations on | a C-terminal | |
| | | | | CH3 domain | SUMO tag. | |
| 2 | ABC057 | AP084 | AP086 | AP004 | AP080 | 1:1:1:1 |
| | (knob/hole) | SEQ ID NO: 8 | SEQ ID NO: 10 | SEQ ID | SEQ ID | |
| | | H chain of | L chain of | NO: 2 | NO: 4 | |
| | | Trastuzumab with | Trastuzumab with | | | |
| | | CH1 replaced by | CL replaced by knob | | | |
| | | hole CH3 domain. | CH3 domain. | | | |
| 3 | ABC060 | AP089 | AP087 | AP004 | AP080 | 1:1:1:1 |
| | (HLA/β2m) | SEQ ID NO: 13 | SEQ ID NO: 11 | SEQ ID | SEQ ID | |
| | | H chain of | L chain of | NO: 2 | NO: 4 | |
| | | Trastuzumab with | Trastuzumab with | | | |
| | | CH1 replaced by | CL replaced by β2m. | | | |
| | | HLA α3 domain. | | | | |
| 4 | ABC061 | AP090 | AP088 | AP004 | AP080 | 1:1:1:1 |
| | (β2m/HLA) | SEQ ID NO: 14 | SEQ ID NO: 12 | SEQ ID | SEQ ID | |
| | | H chain of | L chain of | NO: 2 | NO: 4 | |
| | | Trastuzumab with | Trastuzumab with | | | |
| | | CH1 replaced by | CL replaced by HLA | | | |
| | | β2m. | α3 domain. | | | |
| 5 | ABC131 | AP172 | AP173 | AP004 | AP080 | 1:1:1:1 |
| | (SPIRα/CD47) | SEQ ID NO: 19 | SEQ ID NO: 20 | SEQ ID | SEQ ID | |
| | | H chain of | L chain of | NO: 2 | NO: 4 | |
| | | Trastuzumab with | Trastuzumab with | | | |
| | | CH1 replaced by | CL replaced by | | | |
| | | SPIRα. | CD47. | | | |
| 6 | ABC132 | AP090 | AP182 | AP004 | AP080 | 1:1:1:1 |
| | (β2m/CD1b) | SEQ ID NO: 14 | SEQ ID NO: 26 | SEQ ID | SEQ ID | |
| | | H chain of | L chain of | NO: 2 | NO: 4 | |
| | | Trastuzumab with | Trastuzumab with | | | |
| | | CH1 replaced by | CL replaced by | | | |
| | | β2m. | CD1b α3 domain. | | | |
| 7 | ABC133 | AP183 | AP087 | AP004 | AP080 | 1:1:1:1 |
| | (CD1a/β2m) | SEQ ID NO: 27 | SEQ ID NO: 11 | SEQ ID | SEQ ID | |
| | | H chain of | L chain of | NO: 2 | NO: 4 | |
| | | Trastuzumab with | Trastuzumab with | | | |
| | | CH1 replaced by | CL replaced by β2m. | | | |
| | | CD1a α3 domain. | | | | |
| 8 | ABC570 | AP590 | AP591 | AP008 | AP080 | 1:1:1:1 |
| | ( [symbol] ) | SEQ ID NO: 82 | SEQ ID NO: 83 | SEQ ID NO: 3 | SEQ ID NO: 4 | |
| | | H chain of anti- | L chain of anti-HER2 | H chain of anti- | L chain of | |
| | | HER2 | Trastuzumab with | FAP antibody | anti-FAP | |
| | | Trastuzumab with | CL is replaced by | with knob | antibody with | |
| | | CH1 is replaced by | IgM WT CH2 | mutations on | a C-terminal | |
| | | IgM WT CH2 | domain | CH3 domain | SUMO tag. | |
| | | domain. | | and N297A | | |
| | | | | mutation. | | |
| 9 | ABC571 | AP594 | AP591 | AP008 | AP080 | 1:1:1:1 |
| | knob [symbol] / | SEQ ID NO: 84 | SEQ ID NO: 83 | SEQ ID NO: 3 | SEQ ID NO: 4 | |
| | hole [symbol] | H chain of anti- | L chain of anti-HER2 | H chain of anti- | L chain of | |
| | | HER2 | Trastuzumab with | FAP antibody | anti-FAP | |
| | | Trastuzumab with | CL is replaced by | with knob | antibody with | |
| | | CH1 is replaced by | IgM WT CH2 | mutations on | a C-terminal | |
| | | IgM CH2 domain | domain. | CH3 domain | SUMO tag. | |
| | | containing knob | | and N297A | | |
| | | mutations | | mutation. | | |
| 10 | ABC572 | AP590 | AP595 | AP008 | AP080 | 1:1:1:1 |
| | hole [symbol] / | SEQ ID NO: 82 | SEQ ID NO: 85 | SEQ ID NO: 3 | SEQ ID NO: 4 | |
| | knob [symbol] | H chain of | L chain of anti-HER2 | H chain of anti- | L chain of | |
| | | anti-HER2 | Trastuzumab with | FAP antibody | anti-FAP | |
| | | Trastuzumab with | CL is replaced by | with knob | antibody with | |
| | | CH1 is replaced by | IgM CH2 domain | mutations on | a C-terminal | |
| | | IgM WT CH2 | containing knob | CH3 domain | SUMO tag. | |
| | | domain. | mutations. | and N297A | | |
| | | | | mutation. | | |

[symbol]

indicates data missing or illegible when filed

### Example 2: Orthogonal Fab Pairing Doesn't Require LCs Ratio Optimization (Correct Pairing is Insensitive to LC Ratio)

### Method: Expression and SDS-PAGE Analysis

The CD1s/β2m design with SUMO tag in LC2 was further evaluated with LC1/LC2 plasmid ratio ranging from 3:1 to 1:3, and HC1/HC2 plasmid ratio was fixed at 1:1 with the N297A mutation at both heavy chains. BsAb were produced by co-transfection of 4 plasmids (HC1, LC1, HC2, LC2) with different LC plasmid ratios into Expi293F cells (ThermoFisher #A14527) following the manufacturer's recommendations. Expi293F cells were grown in suspension culture. The supernatant was harvested by centrifugation six days post-transfection and further filtered with 0.22 μm filter. The resultant IgG1 mixture containing BsAb was purified by protein A beads and analyzed under non-reducing and reducing conditions by SDS-PAGE gel.

Materials: the antibodies used in the test and the ratios of HC1:LC1:HC2:LC2 are summarized in Table4

TABLE 4

| | | H chain 1 | L Chain1 | H chain2 | | Molar ratio of |
|---|---|---|---|---|---|---|
| No. | Name | (HC1) | (LC1) | (HC2) | L Chain2(LC2) | HC1:LC1:HC2:LC2 |
| 1 | ABC158 | AP082 | AP081 | AP008 | AP080 | 1:1:1:1 |
| | (WT/WT) | SEQ ID NO: 6 | SEQ ID NO: 5 | SEQ ID NO: 3 | SEQ ID | |
| | | H chain of Trastuzumab | L chain of anti-HER2 | H chain of anti-FAP | NO: 4 | |
| | | with hole mutation on | Trastuzumab | antibody with knob | | |
| | | CH3 and N297A on CH2. | | mutations on CH3 and | | |
| | | | | N297A on CH2. | | |
| 2 | ABC159 | AP090 | AP181 | AP008 | AP080 | 1:1:1:1 |
| | (β2m/CD1a) | SEQ ID NO: 14 | SEQ ID NO: 25 | SEQ ID | SEQ ID | |
| | | H chain of Trastuzumab | L chain of Trastuzumab | NO: 3 | NO: 4 | |
| | | with CH1 replaced by | with CL replaced by | | | |
| | | β2m. | CD1a α3 domain. | | | |
| 3 | ABC160 | AP090 | AP181 | AP008 | AP080 | 1:3:1:1 |
| | (β2m/CD1a) | SEQ ID NO: 14 | SEQ ID NO: 25 | SEQ ID | SEQ ID | |
| | | H chain of Trastuzumab | L chain of Trastuzumab | NO: 3 | NO: 4 | |
| | | with CH1 replaced by | with CL replaced by | | | |
| | | β2m. | CD1a α3 domain. | | | |
| 4 | ABC161 | AP090 | AP181 | AP008 | AP080 | 1:1:1:3 |
| | (β2m/CD1a) | SEQ ID NO: 14 | SEQ ID NO: 25 | SEQ ID | SEQ ID | |
| | | H chain of Trastuzumab | L chain of Trastuzumab | NO: 3 | NO: 4 | |
| | | with CH1 replaced by | with CL replaced by | | | |
| | | β2m. | CD1a α3 domain. | | | |
| 5 | ABC162 | AP090 | AP182 | AP008 | AP080 | 1:1:1:1 |
| | (β2m/CD1b) | SEQ ID NO: 14 | SEQ ID NO: 26 | SEQ ID | SEQ ID | |
| | | H chain of Trastuzumab | L chain of Trastuzumab | NO: 3 | NO: 4 | |
| | | with CH1 replaced by | with CL replaced by | | | |
| | | β2m. | CD1b α3 domain. | | | |
| 6 | ABC163 | AP090 | AP182 | AP008 | AP080 | 1:3:1:1 |
| | (β2m/CD1b) | SEQ ID NO: 14 | SEQ ID NO: 26 | SEQ ID | SEQ ID | |
| | | H chain of Trastuzumab | L chain of Trastuzumab | NO: 3 | NO: 4 | |
| | | with CH1 replaced by | with CL replaced by | | | |
| | | β2m. | CD1b α3 domain. | | | |
| 7 | ABC164 | AP090 | AP182 | AP008 | AP080 | 1:1:1:3 |
| | (β2m/CD1b) | SEQ ID NO: 14 | SEQ ID NO: 26 | SEQ ID | SEQ ID | |
| | | H chain of Trastuzumab | L chain of Trastuzumab | NO: 3 | NO: 4 | |
| | | with CH1 replaced by | with CL replaced by | | | |
| | | β2m. | CD1b α3 domain. | | | |
| 8 | ABC165 | AP183 | AP087 | AP008 | AP080 | 1:1:1:1 |
| | (CD1a/β2m) | SEQ ID NO: 27 | SEQ ID NO: 11 | SEQ ID | SEQ ID | |
| | | H chain of Trastuzumab | L chain of Trastuzumab | NO: 3 | NO: 4 | |
| | | with CH1 replaced by | with CL replaced by | | | |
| | | CD1a α3 domain. | β2m. | | | |
| 9 | ABC166 | AP183 | AP087 | AP008 | AP080 | 1:3:1:1 |
| | (CD1a/β2m) | SEQ ID NO: 27 | SEQ ID NO: 11 | SEQ ID | SEQ ID | |
| | | H chain of Trastuzumab | L chain of Trastuzumab | NO: 3 | NO: 4 | |
| | | with CH1 replaced by | with CL replaced by | | | |
| | | CD1a α3 domain. | β2m. | | | |
| 10 | ABC167 | AP183 | AP087 | AP008 | AP080 | 1:1:1:3 |
| | (CD1a/β2m) | SEQ ID NO: 27 | SEQ ID: NO: 11 | SEQ ID | SEQ ID | |
| | | H chain of Trastuzumab | L chain of Trastuzumab | NO: 3 | NO: 4 | |
| | | with CH1 replaced by | with CL replaced by | | | |
| | | CD1a α3 domain. | β2m. | | | |
| 11 | ABC168 | AP184 | AP087 | AP008 | AP080 | 1:1:1:1 |
| | (CD1b/β2m) | SEQ ID NO: 28 | SEQ ID NO: 11 | SEQ ID | SEQ ID | |
| | | H chain of Trastuzumab | L chain of Trastuzumab | NO: 3 | NO: 4 | |
| | | with CH1 replaced by | with CL replaced by | | | |
| | | CD1b α3 domain. | β2m. | | | |
| 12 | ABC169 | AP184 | AP087 | AP008 | AP080 | 1:3:1:1 |
| | (CD1b/β2m) | SEQ ID NO: 28 | SEQ ID NO: 11 | SEQ ID | SEQ ID | |
| | | H chain of Trastuzumab | L chain of Trastuzumab | NO: 3 | NO: 4 | |
| | | with CH1 replaced by | with CL replaced by | | | |
| | | CD1b α3 domain. | β2m. | | | |
| 13 | ABC170 | AP184 | AP087 | AP008 | AP080 | 1:1:1:3 |
| | (CD1b/β2m) | SEQ ID NO: 28 | SEQ ID NO: 11 | SEQ ID | SEQ ID | |
| | | H chain of Trastuzumab | L chain of Trastuzumab | NO: 3 | NO: 4 | |
| | | with CH1 replaced by | with CL replaced by | | | |
| | | CD1b α3 domain. | β2m. | | | |

### Results:

Genentech's CH1/CL orthogonal Fab format requires chain ratio optimization to ensure the correct HC/LC pairing, as the percentage of correctly assembly BsAbs varies from 63%-84% when the LC1/LC2 ratio varies (Dillon et al., 2017).

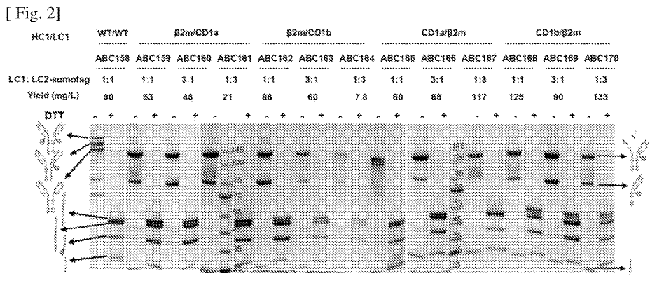

As described above, the engineered LC1 was separated from the rest three-chains assembly on non-reducing SDS-PAGE gel. As shown in FIG. 2, the CD1s/β2m designs showed two dominant bands (~135 kDa corresponds to HC1/HC2/LC2-SUMO and ~25 kDa corresponds to the LC1), no matter whether β2m replaced CH1 or CL. No higher band or lower band compared to the 135 kDa band appeared when changing the LC1/LC2 ratio from 3:1 to 1:3. These results indicate that the LCs of the parental antibodies in our designs can assemble exclusively with their cognate heavy chains.

### Example 3: Characterization of Abio BsAb Format by SEC-HPLC

### Method:

The anti-HER2/FAP BsAb were produced by co-transfection of 4 plasmids (HCl, LC1, HC2, LC2) with varied ratios. The LC2 does not have any SUMO tag and both HCs have the N297A mutation. The desired plasmid combinations were transfected in Expi293F cells and supernatants were purified by protein-Abeads. Eluants were analyzed on reducing and non-reducing SDS-PAGE gel.

SEC-HPLC was used to evaluate the purity and monomer content of the BsAbs, which was performed on an Agilent liquid chromatography instrument installed with AdvanceBio SEC 300A 2.7 μm 4.6×300 mm column (Agilent #PL1580-5301). The flow rate was 0.35 ml/min and the mobile phase was PBS (pH=7.4), and 2.5 μl of protein A-purified BsAbs were injected and eluted isocratic for 15 mins. The elution was monitored by the UV absorbance at 280 nm. The percentage of each species was calculated based on the relative areas of detected peaks by the software.

TABLE 5
Evaluated the assembly of CD1s/β2m or β2m/CD1s BsAb format in the absence of SUMO tag

| | | H chain 1 | L Chain 1 | H chain 2 | L Chain 2 | Molar ratio of |
|---|---|---|---|---|---|---|
| No. | Name | (HC1) | (LC1) | (HC2) | (LC2) | HC1:LC1:HC2:LC2 |
| 1 | ABC171 | AP090 | AP181 | AP008 | AP001 | 1:1:1:1 |
| | β2m/CD1a | SEQ ID NO: 14 | SEQ ID NO: 25 | SEQ ID NO: 3 | SEQ ID NO: 1 | |
| | | H chain of Trastuzumab | L chain of | H chain of anti-FAP | L chain of | |
| | | with CH1 replaced by | Trastuzumab with CL | antibody with knob | anti-FAP | |
| | | β2m, and CH3 domain | replaced by CD1a α3 | mutations on CH3 | antibody. | |
| | | having hole mutations on. | domain. | and N297A on CH2. | | |
| 2 | ABC172 | AP090 | AP182 | AP008 | AP001 | 1:1:1:1 |
| | β2m/CD1b | SEQ ID NO: 14 | SEQ ID NO: 26 | SEQ ID | SEQ ID | |
| | | H chain of Trastuzumab | L chain of | NO: 3 | NO: 1 | |
| | | with CH1 replaced by | Trastuzumab with CL | | | |
| | | β2m. | replaced by CD1b α3 | | | |
| | | | domain. | | | |
| 3 | ABC173 | AP183 | AP087 | AP008 | AP001 | 1:1:1:1 |
| | CD1a/β2m | SEQ ID NO: 27 | SEQ ID NO: 11 | SEQ ID | SEQ ID | |
| | | H chain of Trastuzumab | L chain of | NO: 3 | NO: 1 | |
| | | with CH1 replaced by | Trastuzumab with CL | | | |
| | | CD1a α3 domain. | replaced by β2m. | | | |
| 4 | ABC174 | AP184 | AP087 | AP008 | AP001 | 1:1:1:1 |
| | CD1b/β2m | SEQ ID NO: 28 | SEQ ID NO: 11 | SEQ ID | SEQ ID | |
| | | H chain of Trastuzumab | L chain of | NO: 3 | NO: 1 | |
| | | with CH1 replaced by | Trastuzumab with CL | | | |
| | | CD1b α3 domain. | replaced by β2m. | | | |
| 5 | ABC203 | AP217 | AP218 | AP008 | AP001 | 1:1:1:1 |
| | (anti-FAP/HER2- | SEQ ID NO: 32 | SEQ ID NO: 33 | SEQ ID | SEQ ID | |
| | CrossMab BsAb) | H chain of Trastuzumab | L chain of | NO: 3 | NO: 1 | |
| | | with CH1 replaced CL. | Trastuzumab with CL | | | |
| | | | replaced by CH1. | | | |
| 6 | ABC204 | AP219 | AP220 | AP008 | AP001 | 1:1:1:1 |
| | (TCRβ/TCRα) | SEQ ID NO: 34 | SEQ ID NO: 35 | SEQ ID | SEQ ID | |
| | Wuxibody | H chain of Trastuzumab | L chain of | NO: 3 | NO: 1 | |
| | | with CH1 replaced TCRβ | Trastuzumab with CL | | | |
| | | constant domain. | replaced by TCRα | | | |
| | | | constant domain. | | | |
| 7 | ABC141 | AP215 | AP081 | | | 1:1 |
| | | SEQ ID NO: 31 | SEQ ID NO: 5 | | | |
| | | H chain of Trastuzumab | L chain of anti-HER2 | | | |
| | | | Trastuzumab | | | |
| 8 | ABC089 | | | AP166 | AP164 | 1:1 |
| | | | | SEQ ID NO: 61 | SEQ ID NO: 60 | |
| | | | | H chain of anti- | L chain of | |
| | | | | lysozyme antibody. | anti- | |
| | | | | | lysozyme | |
| | | | | | antibody. | |
| 9 | ABC734 | AP089 | AP087 | AP008 | AT001 | 1:1:1:1 |
| | | SEQ ID NO: 13 | SEQ ID NO: 11 | SEQ ID | SEQ ID | |
| | | H chain of Trastuzumab | L chain of | NO: 3 | NO: 1 | |
| | | with CH1 replaced by | Trastuzumab with CL | | | |
| | | HLA α3 domain. | replaced by β2m. | | | |
| 10 | ABC735 | AP090 | AP088 | AP008 | AP001 | 1:1:1:1 |
| | | SEQ ID NO: 14 | SEQ ID NO: 12 | SEQ ID | SEQ ID | |
| | | H chain of Trastuzumab | L chain of | NO: 3 | NO: 1 | |
| | | with CH1 replaced by | Trastuzumab with CL | | | |
| | | β2m. | replaced by HLA α3 | | | |
| | | | domain. | | | |

### Results:

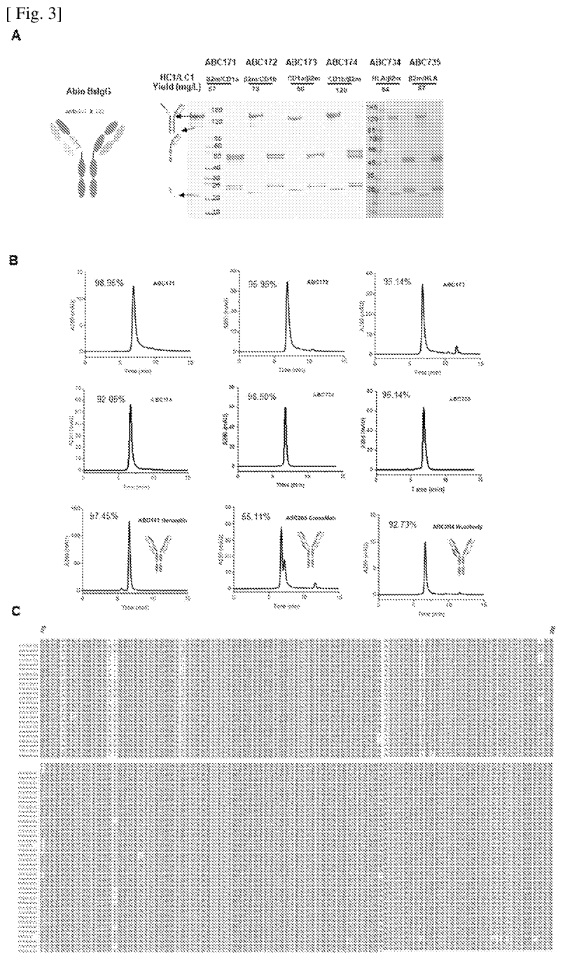

We then evaluated the assembly of CD1s/β2m or β2m/CD1s BsAb format (hereafter the bispecific antibodies in which the CH1/CL of a half body are replace by CD1s/β2m or β2m/CDls are called as Abio BsAbs) without SUMO tag. All the variants displayed expression levels comparable to the parental antibodies. As shown in FIG. 3A, two bands in the non-reducing condition and four bands in the reducing condition were observed as expected in all four designs. The upper band is the engineered-light-chain-missing BsAb (around 130 kDa) and the lower band is the engineered light chain 1 (around 23 kDa). These four CD1s/β2m BsAb were further characterized by SEC-HPLC (FIG. 3B). First of all, the main monomeric species of ABC171, ABC172 ABC173 and ABC174 were eluted at a similar time to the parental mAb. Secondly, Abio BsAbs behave like mAbs with little aggregation. In terms of the monomer BsAb percentages, the Abio BsAbs (~95% monomer) demonstrated comparable quality to the anti-FAP/HER2-CrossMab BsAb (ABC203, 55%) and anti-FAP/HER2-Wuxibody (ABC204, 92%). Although the HLA/β2m (ABC734) or β2m/HLA (ABC735) format showed correct assembly and similar yield as Abio BsAb (FIGS. 3A and 3B), they have higher immunogenicity risk due to the polymorphism of HLA allotypes (FIG. 3C).

### Example 4: Thermal Stability of Abio BsAbs

Method: Differential scanning fluorimetry

Proteins were purified with protein A and size exclusion chromatography (Cytiva #28-9909-44, Superdex 200, 10/300 GL). Differential scanning fluorimetry was performed using QuantStudio 6 Flex (Thermo Fisher) with Protein Thermal Shift™ dye (Thermo #4461146). Briefly, 12.5 μl of protein in PBS with a concentration 0.3-0.5 mg/ml was mixed with 2.5 μl of 8× dye solution (1:1000 dilution in PBS buffer) and 5 μl reaction buffer. The melting curves were measured from 25° C. to 99° C. with a temperature scan rate of 0.05° C./s, and three duplicates were performed. The melting temperatures (Tm) represent peaks when applying a plot of d (fluorescence)/dT to the experimental curve. Tm values of anti-HER2/FAP BsAb variants and the corresponding monoclonal IgG (ABC001 FAP IgG and the ABCO002 FAP IgG N297A) and Wuxibody version of BsAbs (ABC204) were shown in Table 6. Abio bispecifics have comparable Tm1s to bispecifics generated using platforms other than Abio platform.

TABLE 6
Melting temperatures of bispecifics and monoclonals.

| No. | Name | Tₘ1 | Tₘ2 |
|---|---|---|---|
| 1 | ABC171 | 58.04 | 78.82 |
| | β2m/CD1a | | |
| 2 | ABC172 | 58.84 | 78.62 |
| | β2m/CD1b | | |
| 3 | ABC173 | 59.24 | 78.82 |
| | CD1a/β2m | | |
| 4 | ABC174 | 57.64 | 78.62 |
| | CD1b/β2m | | |
| 5 | ABC001 | 71.33 | 78.62 |
| | FAP IgG WT | | |
| 6 | ABC002 | 60.44 | 78.82 |
| | FAP IgG N297A | | |
| 7 | ABC204 | 60.69 | 78.84 |
| | Wuxibody | | |
| 8 | ABC141 | 70.53 | 85.50 |
| 9 | ABC734 | 57.48 | 78.05 |
| | HLA-α3/β2m | | |
| 10 | ABC735 | 59.53 | 78.05 |
| | β2m/HLA-α3 | | |

Tₘ, as used herein, is defined as at which temperature half of the protein is in an unfolded state, which is an important protein property. It is known that deglycosylation at CH2 reduces the Tₘ1. Indeed, anti-FAP hIgG1 with N297A (ABCO002) has a Tₘ1 of 60.4° C., which is lower than the Tₘ1 of 71.33° C. of anti-FAP hIgG1 wt (ABC001) and Tₘ1 of 70.53° C. of anti-HER2 hIgG wt (ABC141).

Firstly, anti-HER2/FAP BsAb variants with N297A (ABC171, ABC172, ABC173, ABC174) have similar Tₘ1 to the parental FAP IgG N297A mAb (ABC002), albeit their Tₘ1 peaks are higher. Tms of these BsAbs are comparable to other human or humanized mAbs varying from 57° C. to 82° C. (Garber and Demarest, 2007). Secondly, our designs have comparable Tₘ1 as the anti-HER2/FAP-Wuxibody format (ABC204) and the anti-HER2/FAP-HLA/β2m format (ABC734 and ABC735). Thirdly, the Tₘ1 of ABC173 (CD1a/β2m design) is slightly higher than ABC171 and ABC172 (β2m/CD1s design).

### Example 5: Mass Spectrometric Analysis of IgG BsAb Showed Correct Chain Assembly

### Method:

Mass spectrometry was performed to evaluate the chain assembly of BsAb. Proteins were purified with protein A and size exclusion chromatography (Cytiva #28-9909-44, Superdex 200, 10/300 GL). Then the proteins were desalted using a C4 column (Waters, ACQUITY UPLC BEH300 C4 1.7 um 2.1*50 mm) with a 10 min gradient elution with phase A (0.1% formic acid) and phase B (0.1% formic and 100% acetonitrile) at a flow rate of 0.3 ml/min on a ACQUITY UPLC system at 80° C. Desalted proteins (~1 mg/ml, 1~3 L) were loaded into an autosampler and analyzed on a Quadrupole time-of-flight (Q-TOF) liquid mass spectrometer (XevoG2-XS QTof, Waters company) with a spray voltage of 3000-3500 V. Data were collected over the m/z range of 500-4000. The raw electrospray spectra were combined, and the multiple charged molecular ions were deconvoluted into a molecular-mass spectrum by using UNIFI software (1.8.2, Waters, MaxEntl Processing). Molecular weights of the light chain and heavy chain components were determined using ExPASy-ProtParam tool.

### Results:

Two mass peaks of ABC171 were detected due to no interchain disulfide within the engineered Fab: one mass matches the engineered anti-HER2 LC1 (theoretical molecular weight is 233340.26 Da, observed molecular weight 23340.00 Da for ABC171), and the other peak matches the molecular weight of the rest molecule (theoretical molecular weight 122622.17 Da, observed molecular weight 122622.25 Da, FIG. 5A). The correct molecular weights were observed for other bispecifics in Abio BsAb format (FIG. 5B-C).

Thus, LC-MS data of all molecules demonstrated that Abio bispecific format ensures the orthogonal LC pairing and no mismatched species were detected.

### Example 6: Dual Binding to Abio BsAb with Comparable Affinity as the Parental Antibodies

### Method: Binding Affinity Assay Measured by Octet

The binding kinetics of the Abio BsAbs to antigens were measured by Bio-Layer Interferometry (BLI) on Octet R8 (Sartorius) at 30° C. and 1000 rpm. Sensors were incubated in assay buffer (PBS 7.4+0.02% Tween 20+0.1% BSA) for at least 10 mins before being loaded into the machine.

Human biotinylated-HER2-Fc (Sino biological #10004-H02H-B) was diluted to 10 ng/ml in assay buffer (PBS 7.4+0.02% Tween20+0.1% BSA) and captured with the Octet Streptavidin (SA) sensor tips (Sartorius ForteBio #18-5019). The baseline was allowed to stabilize for two minutes before sensors were dipped into the antibody solution for two minutes. The association and dissociation were carried out with serial dilutions of BsAbs and mAbs (100 nM, 50 nM, 25 nM, 12.5 nM, 6.25 nM, 3.125 nM, 0 nM). The binding (ka), dissociation (kd) rate constants, and the equilibrium dissociation constant (K_{D}) were determined by fitting all data with a 1:1 binding model using the instrument software.

The dual antigen-binding of Abio BsAbs was also performed on the Octet systems. Human biotinylated-HER2-Fc was immobilized to the Octet Streptavidin (SA) sensor tips as described above. Following a washing step, the loaded biosensors were subjected to association with 100 nM BsAbs for 2 mins, followed by soaking the tips into the second antigen (human FAP, Novoprotein #C14G) for 3 mins. Monoclonal anti-HER2 (Herceptin, ABC141), anti-lysozyme. IgG1. S239D. I332E (ABC089) and assay buffer were used as controls.

### Results:

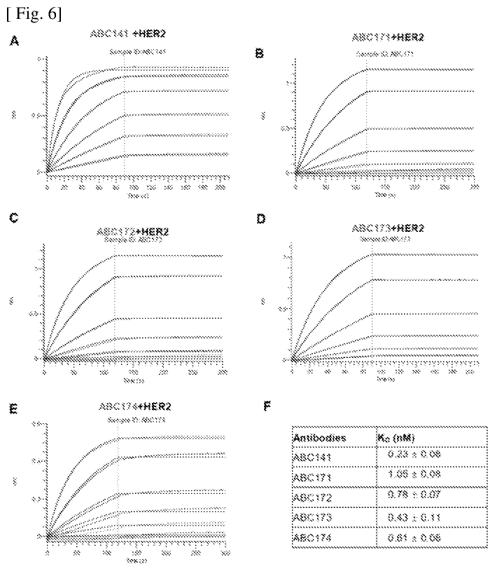

Bio-Layer Interferometry (BLI) was performed to measure the binding affinity of BsAbs to human HER2 (FIG. 6). The four formats of anti-HER2/FAP BsAbs binding to human HER2 are 1.05±0.08 nM, 0.78±0.07 nM, 0.43±0.11 nM, 0.61±0.08 nM, respectively (Table 7), close to the parental Herceptin apparent K_{D} (0.23±0.08 nM) with two Fabs. These results demonstrated that our bispecific designs do not impair the antigen-binding ability of the antibody.

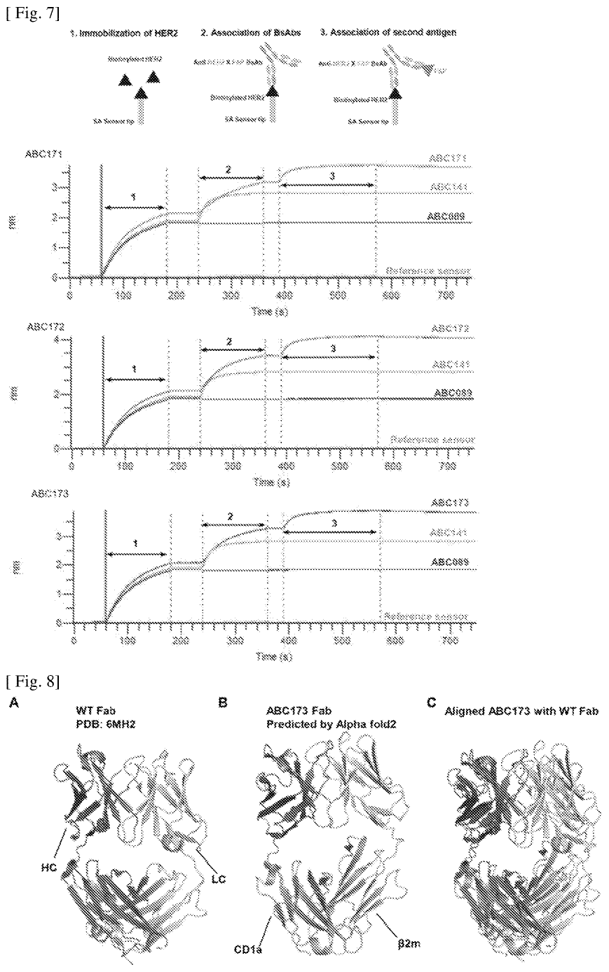

Furthermore, we demonstrated the dual antigen-binding ability of the Abio bispecific format. After the first antigen HER2 loaded on the tip, all BsAbs and Herceptin mAb (ABC141) were associated. Next, the second antigen FAP association occurs for tips loaded with bispecifics, but not Herceptin (FIG. 7). Therefore, the Abio BsAb can simultaneously bind to HER2 and FAP with comparable affinity to the parental antibodies.

TABLE 7
summary of K_{D} values

| Antibodies | K_{D} (nM) |
|---|---|
| ABC141 | 0.23 ± 0.08 |
| ABC171 | 1.05 ± 0.08 |
| ABC172 | 0.78 ± 0.07 |
| ABC173 | 0.43 ± 0.11 |
| ABC174 | 0.61 ± 0.08 |

### Example 7: Predicted Engineered Fab Conformation in ABC173 is Similar to the Wild Type Fab

### Method:

The structure of the engineered Fab of ABC173 was predicted by AlphaFold-multimer and aligned with the crystal structure of Herceptin Fab (PDB: 6MH2). The Root Mean Square Deviation (RMSD) was calculated based on the backbone atoms, and all figures were prepared with PyMOL.

### Results:

We then analyzed the structure of the engineered Fab of ABC173 compared to the original Fab (PDB: 6MH2). As shown in FIGS. 8A and 8B, the engineered Fab of ABC173 has a similar conformation as the WT Fab. Alignment of the backbone atoms of these two structures yielded an RMSD of3.94 Å (FIG. 8C).

### Example 8: Abio BsAb Designs are Broadly Applicable Across Different Antibody Pairs

### Method:

BsAbs were produced by co-transfection of 4 plasmids (HC1, LC1, HC2, LC2) into Expi293F cells (ThermoFisher #A14527) following the manufacturer's recommendations. Both heavy chains contain the N297A mutation. Expi293F cells were grown in suspension culture. The supernatant was harvested by centrifugation six days post-transfection and further filtered with 0.22 m filter. Antibodies were purified with protein A beads from the filtered supernatant. The assembled BsAbs after protein A purification were analyzed by SDS-PAGE. SEC-HPLC was used to evaluate the purity and monomer content of bispecific antibodies. SEC-HPLC analysis was performed on an Agilent liquid chromatography instrument installed with AdvanceBio SEC 300A 2.7 μm 4.6×300 mm column (Agilent Technologies #PL1580-5301). The flow rate was 0.35 ml/min and the mobile phase was PBS (pH=7.4). The elution was monitored by the UV absorbance at 280 nm.

The most favorable format used in ABC173 in terms of chain assembly and purity. Affinities were evaluated across three additional antibody pairs to evaluate if the ABC173 format is broadly applicable. The first pair is anti-mCD20/mCD3 (ABC215), the second pair is anti-HER2/hCD3 (ABC588) and the third pair is anti-hCCR8/hCD3 (ABC591). In mCD20/CD3 bispecific, the CH1/CL of mCD20 was replaced by CD1a/β2m. Bispecifics generated with CrossMab and Wuxibody formats were also tested. All BsAbs were produced by co-expressing four plasmids at the molar ratio of 1:1:1:1 or 2:2:1:1 in Expi293F cells (Table 8).

TABLE 8
Information of BsAbs used in example 8.

| | | H chain 1 | L Chain 1 | H chain 2 | L Chain 2 | Molar ratio of |
|---|---|---|---|---|---|---|
| No. | Name | (HC1) | (LC1) | (HC2) | (LC2) | HC1:LC1:HC2:LC2 |
| 1 | ABC215 | AP265 | AP268 | AP272 | AP271 | 1:1:1:1 |
| | (mCD20/mCD3) | SEQ ID NO: 38 | SEQ ID NO: 41 | SEQ ID NO: 45 | SEQ ID NO: 44 | |
| | (173) | H chain of anti-mouse | L chain of anti-mouse | H chain of anti- | L chain of anti- | |
| | | CD20 with CH1 replaced | CD20 antibody with CL | mouse CD3 with | mouse CD3 | |
| | | by CD1a α3 domain. | replaced by β2m. | hole | antibody. | |
| | | | | mutations in CH3. | | |
| 2 | ABC216 | AP266 | AP269 | AP272 | AP271 | 1:1:1:1 |
| | (mCD20/mCD3) | SEQ ID NO: 39 | SEQ ID NO: 42 | SEQ ID | SEQ ID | |
| | (CrossMab) | H chain of anti-mouse | L chain of anti-mouse | NO: 45 | NO: 44 | |
| | | CD20 with CH1 replaced | CD20 antibody with CL | | | |
| | | by CL domain. | replaced by CH1 domain. | | | |
| 3 | ABC217 | AP267 | AP270 | AP272 | AP271 | 1:1:1:1 |
| | (mCD20/mCD3) | SEQ ID NO: 40 | SEQ ID NO: 43 | SEQ ID | SEQ ID | |
| | (Wuxi) | H chain of anti-mouse | L chain of anti-mouse | NO: 45 | NO: 44 | |
| | | CD20 with CH1 replaced | CD20 antibody with CL | | | |
| | | TCRβ constant domain. | replaced by TCRα constant. | | | |
| 4 | ABC588 | AP183 | AP087 | AP331 | AP330 | 1:1:1:1 |
| | (HER2/hCD3) | SEQ ID NO: 27 | SEQ ID NO: 11 | SEQ ID NO: 47 | SEQ ID NO: 46 | |
| | (173) | H chain of Trastuzumab | L chain of Trastuzumab | H chain of anti- | L chain of anti- | |
| | | with CH1 replaced by | with CH1 replaced by β2m. | human CD3 with | human CD3 | |
| | | CD1a α3 domain. | | hole mutations in | antibody. | |
| | | | | CH3. | | |
| 5 | ABC589 | AP217 | AP218 | AP331 | AP330 | 1:1:1:1 |
| | (HER2/hCD3) | SEQ ID NO: 32 | SEQ ID NO: 33 | SEQ ID | SEQ ID | |
| | (CrossMab) | H chain of Trastuzumab | L chain of Trastuzumab | NO: 47 | NO: 46 | |
| | | with CH1 replaced by | with CL replaced by CH1. | | | |
| | | CL. | | | | |
| 6 | ABC590 | AP219 | AP220 | AP331 | AP330 | 1:1:1:1 |
| | (HER2/hCD3) | SEQ ID NO: 34 | SEQ ID NO: 35 | SEQ ID | SEQ ID | |
| | (Wuxi) | H chain of Trastuzumab | L chain of Trastuzumab | NO: 47 | NO: 46 | |
| | | with CH1 replaced TCRβ | with CL replaced by TCRα | | | |
| | | constant domain. | constant domain. | | | |
| 7 | ABC591 | AP245 | AP248 | AP331 | AP330 | 2:2:1:1 |
| | (hCCR8/hCD3) | SEQ ID NO: 36 | SEQ ID NO: 37 | SEQ ID | SEQ ID | |
| | (173) | H chain of anti- hCCR8 | L chain of anti- hCCR8 | NO: 47 | NO: 46 | |
| | | with CH1 replaced by | antibody with CL replaced | | | |
| | | CD1a α3 domain. | by β2m. | | | |
| 8 | ABC592 | AP246 | AP249 | AP331 | AP330 | 2:2:1:1 |
| | (hCCR8/hCD3) | SEQ ID NO: 58 | SEQ ID NO: 57 | SEQ ID | SEQ ID | |
| | (CrossMab) | H chain of anti- | L chain of anti- hCCR8 | NO: 47 | NO: 46 | |
| | | hCCR8with CH1 | antibody with CL replaced | | | |
| | | replaced by LC. | by CH1. | | | |
| 9 | ABC593 | AP247 | AP220 | AP331 | AP330 | 2:2:1:1 |
| | (hCCR8/hCD3) | SEQ ID NO: 62 | SEQ ID NO: 35 | SEQ ID | SEQ ID | |
| | Wuxi | H chain of anti- hCCR8 | L chain of hCCR8 antibody | NO: 47 | NO: 46 | |
| | | with CH1 replaced by | with CL replaced by TCRα | | | |
| | | TCKβ constant domain. | constant domain. | | | |

### Results:

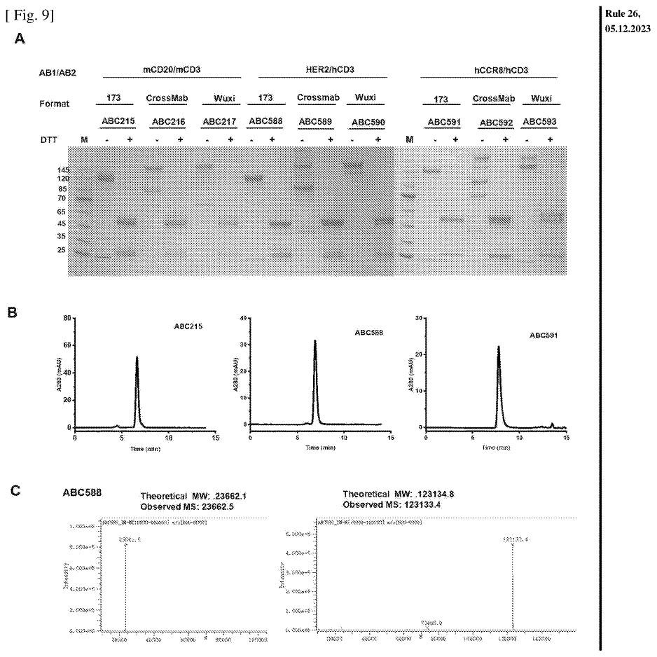

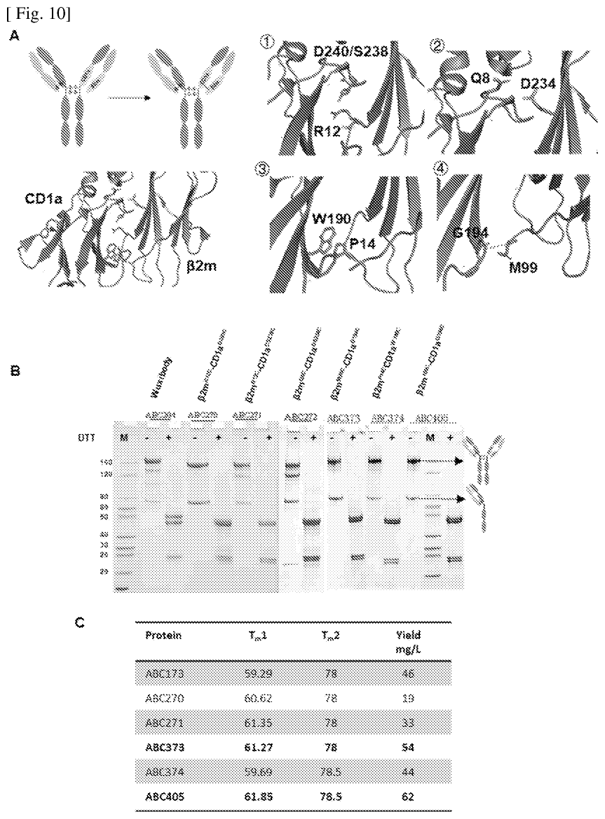

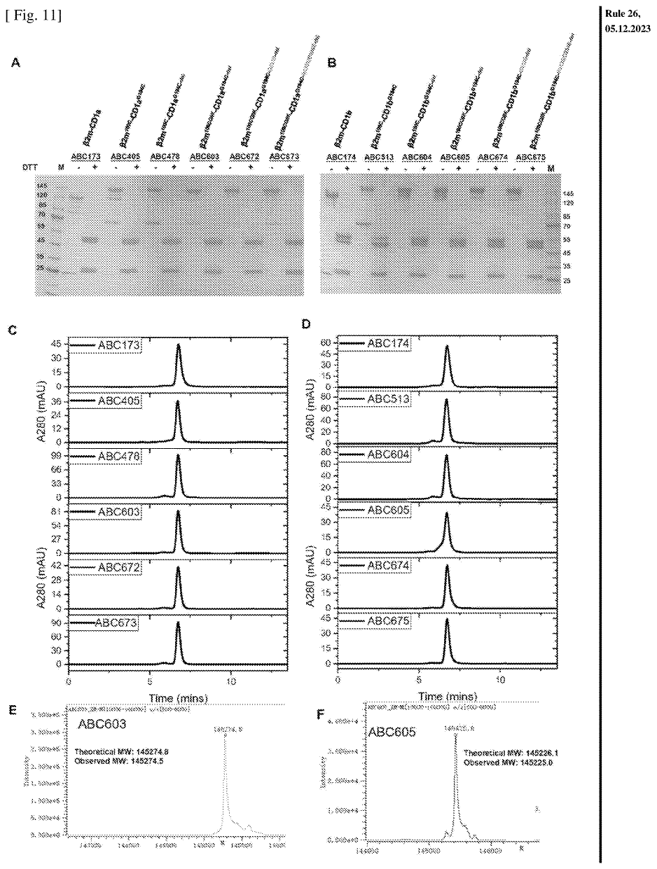

All tested antibody pairs designed in the ABC173 format (ABC215, ABC588 and ABC591) showed expected bands on the non-reducing condition (FIG. 9A), high percentages of monomer (FIG. 9B) and correct molecular weight analyzed by LC-MS. In FIG. 9C, the intact molecule weight of ABC588 measured by mass spectrometry matches the theoretical molecular weight, and mispaired species are not detected. BsAbs in the ABC173 format seem to have higher purity than the corresponding BsAbs in CrossMab or the Wuxibody format (FIG. 9A).

### Example 9: Further Engineering on Abio Bispecific Antibodies Achieved Improved Thermal Stability

Methods: Mutations based on the ABC173 format and ABC174 format were introduced by standard site-directed mutagenesis. Variants were expressed and purified the same as described in example 3. The chain ratio of LC1:HC1:LC2:HC2 is 1:1:1:1 or 2:2:1:1. After protein A beads purification, the eluted proteins were analyzed by non-reducing and reducing SDS-PAGE to examine the formation of four-chain BsAb. SEC-HPLC was used to evaluate the purity and monomer content of the mutants. SEC-HPLC analysis was performed on an Agilent liquid chromatography instrument installed with AdvanceBio SEC 300A 2.7 μm 4.6×300 mm column (Agilent Technologies #PL1580-5301). The flow rate was 0.35 ml/min and the mobile phase was PBS (pH=7.4). The elution was monitored by the UV absorbance at 280 nm. Differential scanning fluorimetry was performed as described above in example 4.

### Results:

To improve the biophysical properties of the Abio bispecific antibodies, the interface between the CD1a α3 domain and β2m was subjective to mutagenesis, and six pairs of cysteine mutations were tested (FIG. 10A). The numbering is based on the crystal structure of CD1a/β2mPDB ID 1XZO). The detailed chain information of CD1a^{D240C}/β2m^{R12C} (ABC270), CD1a^{S238C}/β2m^{R12C} (ABC271), CD1a^{D234C}/β2m^{Q8C} (ABC272), CD1a^{G194C}/β2m^{M99C} (ABC373), CD1a^{W190C}/β2m^{P14C} (ABC374) and CD1a^{G194C}/β2m^{100C} (ABC405) variants are summarized in Table 9.

TABLE 9

| | | | | | | Molar |
|---|---|---|---|---|---|---|
| | | H chain 1 | L Chain 1 | H chain 2 | L Chain 2 | ratio of |
| No. | Name | (HC1) | (LC1) | (HC2) | (LC2) | HC1:LC1:HC2:LC2 |
| 1 | ABC270 | AP254 | AP253 | AP008 | AP001 | 1:1:1:1 |
| | (CD1a [symbol] / | SEQ ID NO: 66 | SEQ ID NO: 65 | SEQ ID | SEQ ID | |
| | β2m [symbol] ) | H chain of Trastuzumab with CH1 | L chain of | NO: 3 | NO: 1 | |
| | | replaced by CD1aD240C domain. | Trastuzumab with CL | | | |
| | | | replaced byβ2mR12C | | | |
| | | | domain. | | | |
| 2 | ABC271 | AP255 | AP253 | AP008 | AP001 | 1:1:1:1 |
| | (CD1a [symbol] / | SEQ ID NO: 67 | SEQ ID NO: 65 | SEQ ID | SEQ ID | |
| | β2m [symbol] ) | H chain of Trastuzumab with CH1 | L chain of | NO: 3 | NO: 1 | |
| | | replaced by CD1aS238C domain. | Trastuzumab with CL | | | |
| | | | replaced by β2mR12C | | | |
| | | | domain. | | | |
| 3 | ABC272 | AP256 | AP257 | AP008 | AP001 | 1:1:1:1 |
| | (CD1a [symbol] / | SEQ ID NO: 68 | SEQ ID NO: 69 | SEQ ID | SEQ ID | |
| | β2m [symbol] ) | H chain of Trastuzumab with CH1 | L chain of | NO: 3 | NO: 1 | |
| | | replaced by CD1aD234C domain. | Trastuzumab with CL | | | |
| | | | replaced byβ2mQ8C | | | |
| | | | domain. | | | |
| 4 | ABC373 | AP448 | AP446 | AP008 | AP001 | 1:1:1:1 |
| | (CD1a [symbol] / | SEQ ID NO: 73 | SEQ ID NO: 71 | SEQ ID | SEQ ID | |
| | β2m [symbol] ) | H chain of Trastuzumab with CH1 | L chain of | NO: 3 | NO: 1 | |
| | | replaced by CD1aG194C domain. | Trastuzumab with CL | | | |
| | | | replaced byβ2mM99C | | | |
| | | | domain. | | | |
| 5 | ABC374 | AP447 | AP445 | AP008 | AP001 | 1:1:1:1 |
| | (CD1a [symbol] / | SEQ ID NO: 72 | SEQ ID NO: 70 | SEQ ID | SEQ ID | |
| | β2m [symbol] ) | H chain of Trastuzumab with CH1 | L chain of | NO: 3 | NO: 1 | |
| | | replaced by CD1aW190C domain. | Trastuzumab with CL | | | |
| | | | replaced byβ2mP14C | | | |
| | | | domain. | | | |
| 6 | ABC405 | AP448 | AP450 | AP008 | AP001 | 1:1:1:1 |
| | (CD1a [symbol] / | SEQ ID NO: 73 | SEQ ID NO: 74 | SEQ ID | SEQ ID | |
| | β2m [symbol] )* | H chain of Trastuzumab with CH1 | L chain of | NO: 3 | NO: 1 | |
| | | replaced by CD1aG194C domain. | Trastuzumab with CL | | | |
| | | | replaced by β2m | | | |
| | | | insert100C domain. | | | |
| 7 | ABC478 | AP520 | AP450 | AP008 | AP001 | 1:1:1:1 |
| | (CD1a [symbol] / | SEQ ID NO: 76 | SEQ ID NO: 74 | SEQ ID | SEQ ID | |
| | β2m [symbol] )* | H chain of Trastuzumab with CH1 | L chain of | NO: 3 | NO: 1 | |
| | | replaced by CD1aG1940-del | Trastuzumab with CL | | | |
| | | domain. | replaced by β2m- | | | |
| | | | 100C domain. | | | |
| 8 | ABC603 | AP520 | AP483 | AP008 | AP001 | 2:2:1:1 |
| | (CD1a [symbol] / | SEQ ID NO: 76 | SEQ ID NO: 75 | SEQ ID | SEQ ID | |
| | β2m [symbol] )* | H chain of Trastuzumab with CH1 | L chain of | NO: 3 | NO: 1 | |
| | | replaced by CD1aG1940-del | Trastuzumab with CL | | | |
| | | domain. | replaced by | | | |
| | | | β2m100C/Q8K | | | |
| | | | domain. | | | |
| 9 | ABC672 | AP716 | AP483 | AP008 | AP001 | 2:2:1:1 |
| | (CD1a [symbol] / | SEQ ID NO: 77 | SEQ ID NO: 75 | SEQ ID | SEQ ID | |
| | β2m [symbol] )* | H chain of Trastuzumab with CH1 | L chain of | NO: 3 | NO: 1 | |
| | | replaced by | Trastuzumab with CL | | | |
| | | CD1aG194C/G233S/del domain. | replaced by | | | |
| | | | β2m100C/Q8K | | | |
| | | | domain. | | | |
| 10 | ABC673 | AP717 | AP483 | AP008 | AP001 | 2:2:1:1 |
| | (CD1a [symbol] / | SEQ ID NO: 78 | SEQ ID NO: 75 | SEQ ID | SEQ ID | |
| | β2m [symbol] )* | H chain of Trastuzumab with CH1 | L chain of | NO: 3 | NO: 1 | |
| | | replaced by CD1aG194C-G233S- | Trastuzumab with CL | | | |
| | | D234E-del domain. | replaced by β2m | | | |
| | | | insert100C/Q8K | | | |
| | | | domain. | | | |
| 11 | ABC513 | AP562 | AP450 | AP008 | AP001 | 1:1:1:1 |
| | (CD1b [symbol] / | SEQ ID NO: 79 | SEQ ID NO: 74 | SEQ ID | SEQ ID | |
| | β2m [symbol] )* | H chain of Trastuzumab with CH1 | L chain of | NO: 3 | NO: 1 | |
| | | replaced by CD1bG194C domain. | Trastuzumab with CL | | | |
| | | | replaced by β2m 100C | | | |
| | | | domain. | | | |
| 12 | ABC604 | AP563 | AP450 | AP008 | AT001 | 2:2:1:1 |
| | (CD1a [symbol] / | SEQ ID NO: 80 | SEQ ID NO: 74 | SEQ ID | SEQ ID | |
| | β2m [symbol] )* | H chain of Trastuzumab with CH1 | L chain of | NO: 3 | NO: 1 | |
| | | replaced by CD1aG194C-del | Trastuzumab with CL | | | |
| | | domain. | replaced by β2m 100C | | | |
| | | | domain. | | | |
| 13 | ABC605 | AP563 | AP483 | AP008 | AP001 | 2:2:1:1 |
| | (CD1b [symbol] / | SEQ ID NO: 80 | SEQ ID NO: 75 | SEQ ID | SEQ ID | |
| | β2m [symbol] )* | H chain of Trastuzumab with CH1 | L chain of | NO: 3 | NO: 1 | |
| | | replaced CD1a6194C-del | Trastuzumab with CL | | | |
| | | domain. | replaced by β2m | | | |
| | | | 100C/Q8K domain. | | | |
| 14 | ABC674 | AP564 | AP483 | AP008 | AP001 | 2:2:1:1 |
| | (CD1b [symbol] / | SEQ ID NO: 81 | SEQ ID NO: 75 | SEQ ID | SEQ ID | |
| | β2m [symbol] )* | H chain of Trastuzumab with CH1 | L chain of | NO: 3 | NO: 1 | |
| | | replaced by CD1bG194C-G233S- | Trastuzumab with CL | | | |
| | | del domain. | replaced by | | | |
| | | | β2m100C/Q8K | | | |
| | | | domain. | | | |
| 15 | ABC675 | AP565 | AP483 | AP008 | AP001 | 2:2:1:1 |
| | (CD1b [symbol] / | SEQ ID NO: 59 | SEQ ID NO: 75 | SEQ ID | SEQ ID | |
| | β2m [symbol] )* | H chain of Trastuzumab with CH1 | L chain of | NO: 3 | NO: 1 | |
| | | replaced by CD1bG194C-G233S- | Trastuzumab with CL | | | |
| | | D234E-del domain. | replaced by | | | |
| | | | β2m100C/Q8K | | | |
| | | | domain. | | | |
| 16 | ABC736 | AP801 | AP483 | AP004 | AP001 | 2:2:1:1 |
| | (CD1a [symbol] / | SEQ ID NO: 86 | SEQ ID NO: 75 | SEQ ID | SEQ ID | |
| | β2m [symbol] ) | H chain of Trastuzumab with CH1 | L chain of | NO: 2 | NO: 1 | |
| | | replaced by CD1aG194C-del | Trastuzumab with CL | | | |
| | | domain. | replaced by | | | |
| | | | β2m100C-Q8K | | | |
| | | | domain. | | | |
| 17 | ABC737 | AP802 | AP483 | AP004 | AP001 | 2:2:1:1 |
| | (CD1a [symbol] / | SEQ ID NO: 87 | SEQ ID NO: 75 | SEQ ID | SEQ ID | |
| | β2m [symbol] ) | H chain of Trastuzumab with CH1 | L chain of | NO: 2 | NO: 1 | |
| | | replaced by CD1aG194C-G233S- | Trastuzumab with CL | | | |
| | | D234E-del domain. | replaced by β2m | | | |
| | | | insert100C/Q8K | | | |
| | | | domain. | | | |
| 18 | ABC738 | AP803 | AP483 | AP004 | AP001 | 2:2:1:1 |
| | (CD1b [symbol] / | SEQ ID NO: 88 | SEQ ID NO: 75 | SEQ ID | SEQ ID | |
| | β2m [symbol] ) | H chain of Trastuzumab with CH1 | L chain of | NO: 2 | NO: 1 | |
| | | replaced by CD1aG194C-del | Trastuzumab with CL | | | |
| | | domain. | replaced by | | | |
| | | | β2m [symbol] domain. | | | |
| 19 | ABC739 | AP804 | AP483 | AP004 | AP001 | 2:2:1:1 |
| | (CD1b [symbol] / | SEQ ID NO: 89 | SEQ ID NO: 75 | SEQ ID | SEQ ID | |
| | β2m [symbol] ) | H chain of Trastuzumab with CH1 | L chain of | NO: 2 | NO: 1 | |
| | | replaced by | Trastuzumab with CL | | | |
| | | CD1bG194C/G233S/D234E-del | replaced by | | | |
| | | domain. | β2m [symbol] domain. | | | |
| 20 | ABC747 | AP848 | AP483 | | | 1:1 |
| | (Fab of Herceptin with CH/CL | SEQ ID NO: 108 | SEQ ID | | | |
| | replaced by CD1a [symbol] | Fd of Herceptin Fab with CH1 | NO: 75 | | | |
| | β2m [symbol] ) | replaced by CD1aG194C-del-his | | | | |
| | | tag domain. | | | | |
| 21 | ABC748 | AP849 | AP483 | | | 1:1 |
| | (Fab of Herceptin with CH/CL | SEQ ID NO: 109 | SEQ ID | | | |
| | replaced by CD1a [symbol] | Fd of Herceptin Fab with CH1 | NO: 75 | | | |
| | β2m [symbol] ) | replaced by CD1aG194C-G2338- | | | | |
| | | deLVPRG-Fd-His | | | | |
| 22 | ABC749 | AP850 | AP483 | | 1:1 | |
| | (Fab of Herceptin with CH/CL | SEQ ID NO: 110 | SEQ ID | | | |
| | replaced by CD1a [symbol] | Fd of Herceptin Tab with CH1 | NO: 75 | | | |
| | β2m [symbol] ) | replaced by CD1aG194C-G233S- | | | | |
| | | D234E-deLVPRG-Fd-His | | | | |
| 23 | ABC750 | AP851 | AP081 | | | 1:1 |
| | (Fab of Herceptin) | SEQ ID NO: 111 | SEQ ID | | | |
| | | Fd of Herceptin Fah with His tag | NO: 5 | | | |

[symbol]

indicates data missing or illegible when filed

*β2m^{100C} means adding additional cysteine in the end of β2m sequence.

As shown in FIG. 10B, the fully assembled four-chain bispecific antibody with 150 kDa molecular weight appeared in all variants, in which cysteine mutations were introduced in the interface of CD1a/β2m. Although disulfide bonds were formed in all the designed molecules, the efficiency of the disulfide bond formation and the homogeneity varies.

TABLE 10

| | | | ΔTₘ₁ | | ΔTₘ₂ | |
|---|---|---|---|---|---|---|
| | | | (compared to | | (compared to | ΔYield/ |
| No. | Protein | Tₘ₁ | ABC173) | Tₘ₂ | ABC173) | Yield173% |
| 1 | ABC173 | 59.3 | 0 | 78 | 0 | 0.0% |
| 2 | ABC270 | 60.6 | 1.3 | 78 | 0 | −58.7% |
| | (CD1a [symbol] /β2m [symbol] ) | | | | | |
| 3 | ABC271 | 61.4 | 2.1 | 78 | 0 | −28.3% |
| | (CD1a [symbol] /β2m [symbol] ) | | | | | |
| 5 | ABC373 | 61.3 | 2.0 | 78 | 0 | 17.4% |
| | (CD1a [symbol] /β2m [symbol] ) | | | | | |
| 6 | ABC374 | 59.7 | 0.4 | 78.5 | 0.5 | −4.3% |
| | (CD1a [symbol] /β2m [symbol] ) | | | | | |
| 7 | ABC405 | 61.9 | 2.6 | 78.5 | 0.5 | 34.8% |
| | (CD1a [symbol] /β2m [symbol] ) | | | | | |

[symbol]

indicates data missing or illegible when filed

The thermal stabilities (Tₘ1 and Tₘ2) and yields for the antibodies are summarized in Table 10. All six pairs of cysteine mutations in CD1s/β2m showed increased Tₘ1, compared to the original molecules ABC173, while Tₘ2 remained the same. These results indicate that introducing cysteine mutations in the heterodimerization domain of CD1s/β2m improved thermal stability. Mutation pair of CD1a^{G194C}/β2m^{100C} (ABC405) has improved Tₘ1 by 2.6° C.

Second, it is likely that mutagenesis can affect the yield and solubility of the engineered bispecifics. For example, the expression of ABC270 is dramatically reduced (FIG. 10B and 10C, ABC270), while the yield of ABC405 is higher than the parent ABC173. Therefore, the bispecific format of ABC405 is the most favorable design regarding expression yield and thermal stability.

In the previous bispecific molecules, there are amino acids (GSLVPRG) that do not belong to the CD1a α3 domain at the C-terminus of the CD1a α3 domain. To reduce the immunogenicity, amino acids LVPRG were depleted, leaving GS as a linker between the CD1a α3 domain and the CH2 domain. The resulting molecule (ABC478) derived from ABC405 has similar thermal stability as ABC405.

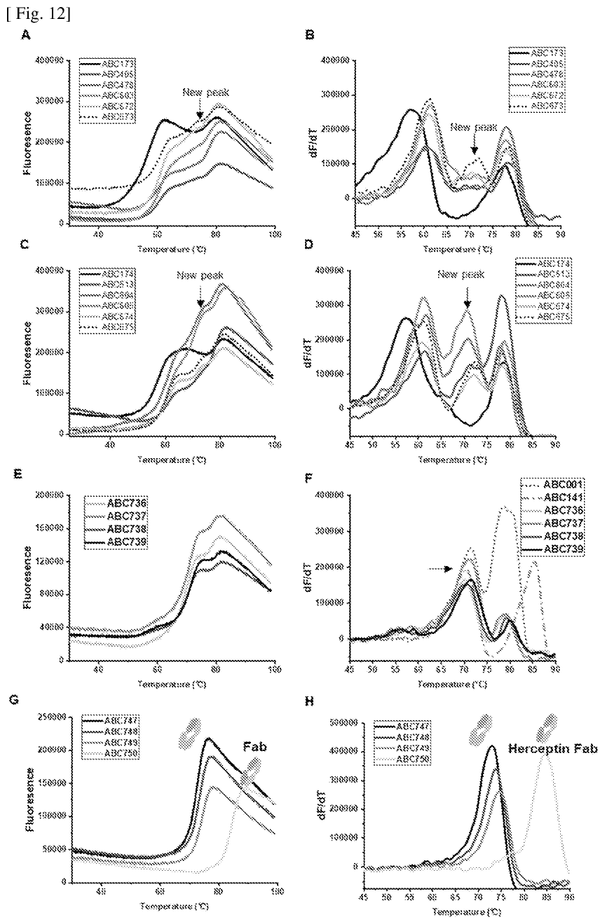

Based on the format ABC478, we performed a second round of optimization to increase the yield and stability of the bispecific antibody. Mutations are introduced to improve the interaction between CD1s α3 domain and β2m by either forming hydrogen bonds and/or salt bridge interactions (ABC603, ABC672, ABC673). As shown in FIG. 11A, all variants showed an expected band of 150 kDa and similar Tₘ1 as ABC478 (Table 11). All these variants showed little aggregates as indicated by HPLC-SEC (FIG. 11C).

The same strategies were applied to engineer ABC174 format. Variants derived from ABC174 formed desired four-chain antibody as shown by the band of 150 kDa by non-reducing SDS-PAGE (FIGS. 11B and 11D).

TABLE 11

| | | | | ATₘ₁ |
|---|---|---|---|---|
| No. | Protein | ratio | Tₘ₁ | (compared to ABC173) |
| 1 | ABC173 | 1:1:1:1 | 59.29 | 0 |
| 2 | ABC405 | 1:1:1:1 | 61.80 | 2.51 |
| | (CD1a^{G194C}/β2m^{100C}) | | | |
| 3 | ABC478 | 1:1:1:1 | 61.50 | 2.21 |
| | (CD1a^{G194C-del}/β2m^{100C}) | | | |
| 4 | ABC603 | 2:2:1:1 | 61.87 | 2.58 |
| | (CD1a^{G194C-del}/β2m^{100C/Q8K}) | | | |
| 5 | ABC672 | 2:2:1:1 | 61.50 | 2.21 |
| | (CD1a^{G194C/G233S-del}/β2m^{100C/Q8K}) | | | |
| 6 | ABC673 | 2:2:1:1 | 61.50 | 2.21 |
| | (CD1a^{G194C/G233S/D234E-Del}/β2m^{100C/Q8K}) | | | |
| 7 | ABC736 | 2:2:1:1 | 70.00 | 10.71 |
| | (CD1a^{G194C-Del}/β2m^{100C/Q8K}) | | | |
| 8 | ABC737 | 2:2:1:1 | 71.00 | 11.71 |
| | (CD1a^{G194C/G233S/D234E-Del}/β2m^{100C/Q8K}) | | | |
| 9 | ABC174 | 1:1:1:1 | 57.29 | 0 |
| 10 | ABC513 | 1:1:1:1 | 61.00 | 3.71 |
| | (CD1a^{G194C}/β2m^{100C}) | | | |
| 11 | ABC604 | 2:2:1:1 | 61.57 | 4.28 |
| | (CD1a^{G194C-Del}/β2m^{100C}) | | | |
| 12 | ABC605 | 2:2:1:1 | 61.57 | 4.28 |
| | (CD1b^{G194C-Del}/β2m^{100C/Q8K}) | | | |
| 13 | ABC674 | 2:2:1:1 | 61.33 | 4.04 |
| | (CD1b^{G194C/G233S-Del}/β2m^{100C/Q8K}) | | | |
| 14 | ABC675 | 2:2:1:1 | 61.15 | 3.86 |
| | (CD1b^{G194C/G233S/D234E-Del}/β2m^{100C/Q8K}) | | | |
| 15 | ABC738 | 2:2:1:1 | 71.00 | 13.71 |
| | (CD1b^{G194C-Del}/β2m^{100C/Q8K}) | | | |
| 16 | ABC739 | 2:2:1:1 | 71.50 | 14.21 |
| | (CD1b^{G194C/G233S/D234E-Del}/β2m^{100C/Q8K}) | | | |
| 17 | ABC747 | 1:1 | 72.8 | — |
| | (Fab of Herceptin with CH/CL replaced by | | | |
| | (CD1a^{G194C-Del}/β2m^{100C/Q8K}) | | | |
| 18 | ABC748 | 1:1 | 74.2 | |
| | (Fab of Herceptin with CH/CL, replaced by | | | |
| | (CD1a^{G194C/G233S-Del}/β2m^{100C/Q8K}) | | | |
| 19 | ABC749 | 1:1 | 74.8 | |
| | (Fab of Herceptin with CH/CL replaced by | | | |
| | (CD1a^{G194C/G233S/D234E-Del}/β2m^{100C/Q8K}) | | | |
| 20 | ABC750 | 1:1 | 85.5 | |
| | (Fab of Herceptin) | | | |

As shown in Table 11, all variants of the ABC173 and ABC174 showed good stabilities. In particular, Tₘ1 of the ABC173-derived designs is improved by 2.2° C. to 2.6° C. compared to ABC173. And the ABC174-derived molecules improve Tₘ1 by 3.7~4.3° C. compared to ABC174. Among those molecules, ABC405, ABC478, ABC603, ABC672, and ABC673 have the most favorable biophysical properties in expression and thermal stability.

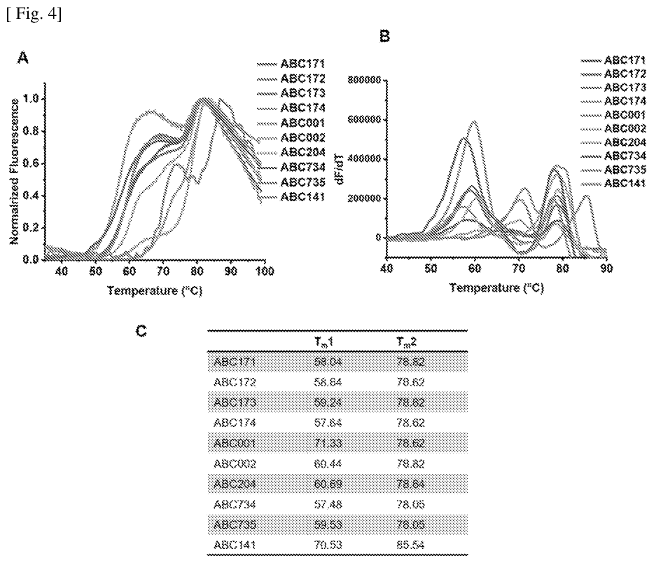

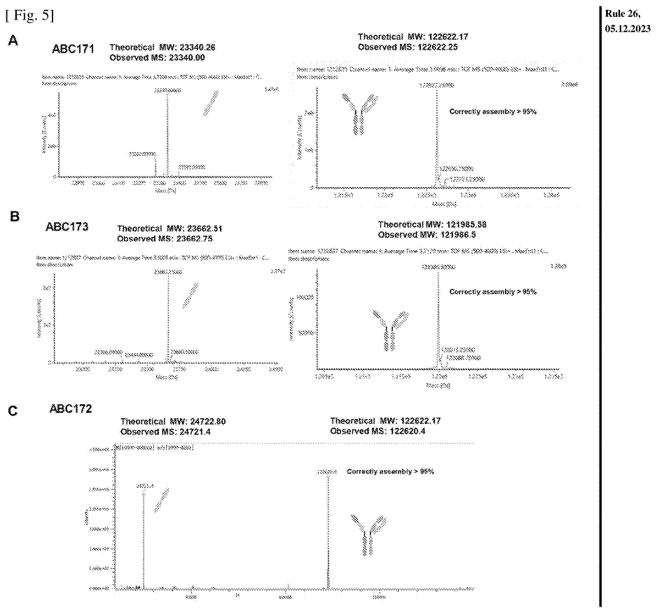

Next, BsAbs (ABC736, ABC737, ABC738 and ABC739) with wild-type CH2 were constructed and the thermostability of these constructs was measured. Tₘ1s of bispecifics are ~71° C., and Tₘ1s of the parental anti-FAP and anti-HER2 hIgG WT is also ~71° C. (FIG. 4). Therefore, the engineered bispecific antibody is as stable as natural antibodies.

In order to determine which melting peak corresponds to the unfolding of which domain, Fab variants (ABC747, ABC748 and ABC749) of ABC603, ABC673 and ABC675 were constructed and compared to the WT Fab of Herceptin (ABC750). The Tₘ of Fab variants in which the CH1/CL is replaced by engineered CD1s/β2m is ~71° C., which is similar to the Tₘ of the CH2 domain, while the Tₘ of Herceptin Fab WT is 85.5° C. (FIGS. 12G and 12H). Therefore, the first melting peak of bispecifics is the unfolding peak of CH2 domain coupled with the engineered Herceptin Fab.

Compared to MHC/β2m in WO2022166728, our Abio BsAb format has the following advantages: 1) bispecifics in our Abio platform will have less immunogenicity, as CD1s are nonpolymorphic. 2) The Tₘ1 of Abio BsAbs molecules with engineered CD1a/β2m is about 71° C., which is as stable as mAb hIgG1, while the reported Tₘ1 of MHL147-33322-IgG1-F118A is 67° C.

### Example 10: Cell Based-Binding Affinity of Abio Bispecific Antibodies with Comparable Affinity as the Parental Antibodies

### Methods: Cell-Based Binding Assays to Determine the Binding Affinity of BsAbs

BsAbs and parental antibodies were expressed and purified as described in example 3. The chain ratio of LC1:HC1:LC2:HC2 is 2:2:1:1. All antibodies were purified first by protein A and then by SEC.

Antibody binding to antigen was measured by FACS: Antigen-expressing cells were aliquoted to 0.3 million per well in 96-U-bottom plate, and incubated first with 50 μl Fc Receptor blocking solution (Human TruStain FcX™, BioLegend #422302, 1:40 dilution) on ice for 15 mins. Serially diluted antibodies or bispecifics were then added, with a maximum final concentration of 1000 nM and 3-fold dilution using PBS buffer. After incubating on ice for 30 mins, the plates were washed two times with FACS buffer (PBS buffer with 0.5 mg/ml BSA, 0.02% tween) and incubated with FITC anti-human IgG antibody (Goat Anti Human (IgG Fc) secondary antibody FITC, Abcam #ab97224, 1:1000) on ice for 30 mins, followed by two times wash with FACS buffer. Next, cells were stained with 7-AAD (Viability Staining Solution, Invitrogen #00-6993-50) and resuspended in 150 μl FACS buffer before flow cell analysis (Cytek®Aurora). Data were analyzed with GraphPad Prism. Median fluorescent intensity was plotted versus antibody concentration and EC50 was calculated by fitting the curve with the function of log (agonist) vs. response—Variable slope (four parameters).

### Materials:

Cell lines: cell line SK-BR-3 (SKBR3, HER2 antigen high expressing cell line, ATCC #HTB-30), Jurkat clone E6-1 cell line (express hCD3 antigen, ATCC 190 TIB-152), hCCR8-transfected 293T cell line.

TABLE 12

| | | | | | | Molar |
|---|---|---|---|---|---|---|
| | | H chain 1 | L Chain 1 | H chain 2 | L Chain 2 | ratio of |
| No. | Name | (HC1) | (LC1) | (HC2) | (LC2) | HC1:LC1:HC2:LC2 |
| 1 | ABC138 | AP016 | AP040 | | | 1:1 |
| | (anti- | SEQ ID NO: 101 | SEQ ID NO: 102 | | | |
| | hCCR8) | H chain of anti-hCCR8 antibody | L chain of anti-hCCR8 | | | |
| | | | antibody | | | |
| 2 | ABC723 | AP520 | AP483 | AP331 | AP330 | 2:2:1:1 |
| | | SEQ ID NO: 76 | SEQ ID NO: 75 | SEQ ID NO: 47 | SEQ ID NO: 46 | |
| | | H chain of Trastuzumab with CH1 | L chain of Trastuzumab | H chain of anti-human | L chain of anti- | |
| | | replaced by CD1a [symbol] domain. | with CL replaced by | CD3 with hole | human CD3 | |
| | | | β2m [symbol] domain. | mutations in CH3. | antibody. | |
| 3 | ABC724 | AP565 | AP483 | AP331 | AP330 | 2:2:1:1 |
| | | SEQ ID NO: 59 | SEQ ID NO: 75 | SEQ ID NO: 47 | SEQ ID NO: 46 | |
| | | H chain of Trastuzumab with CH1 | L chain of Trastuzumab | | | |
| | | replaced by CD1b [symbol] | with CL replaced by | | | |
| | | domain. | β2m [symbol] domain. | | | |
| 4 | ABC722* | | | AP239 | AP164 | 1:1 |
| | | | | SEQ ID NO: 103 | SEQ ID NO: 60 | |
| | | | | H chain of anti- | L chain of anti- | |
| | | | | lysozyme antibody | lysozyme | |
| | | | | with N297A | antibody. | |
| | | | | mutation. | | |
| 5 | ABC728 | | | AP794: | AP330: | 1:1 |
| | | | | SEQ ID NO: 104 | SEQ ID NO: 46 | |
| | | | | H chain of anti-human | | |
| | | | | CD3 antibody | | |
| 6 | ABC752 | AP853 | AP773 | AP331 | AP330 | 2:2:1:1 |
| | | SEQ ID NO: 106 | SEQ ID NO: 105 | SEQ ID NO: 47 | SEQ ID NO: 46 | |
| | | H chain of anti- hCCR8 with CH1 | L chain of anti-hCCR8 | | | |
| | | replaced by CD1a | antibody with CL | | | |
| | | α3 [symbol] domain. | replaced by β2m [symbol] . | | | |
| 7 | ABC753 | AP854 | AP773 | AP331 | AP330 | 2:2:1:1 |
| | | SEQ ID NO: 107 | SEQ ID NO: 105 | SEQ ID NO: 47 | SEQ ID NO: 46 | |
| | | H chain of anti- hCCR8 with CH1 | | | | |
| | | replaced by CD1b | | | | |
| | | α3 [symbol] domain. | | | | |

[symbol]

indicates data missing or illegible when filed

*ABC722 is a negative control antibody.

### Results:

The formats with improved thermal stability, such as ABC603, ABC605, ABC673 and ABC675, were used to generate two additional antibody pairs to evaluate if they are broadly applicable. Mutations based on ABC603 and ABC605 format were introduced to ABC588 to generate ABC723 (ABC723_HER2/hCD3_603) and ABC724 (ABC724_HER2/hCD3_605), respectively. Similarly, mutations based on ABC673 and ABC675 format were introduced to ABC591 to generate ABC752 (ABC752_hCCR8/hCD3_673) and ABC753 (ABC753_hCCR8/hCD3_675), respectively.

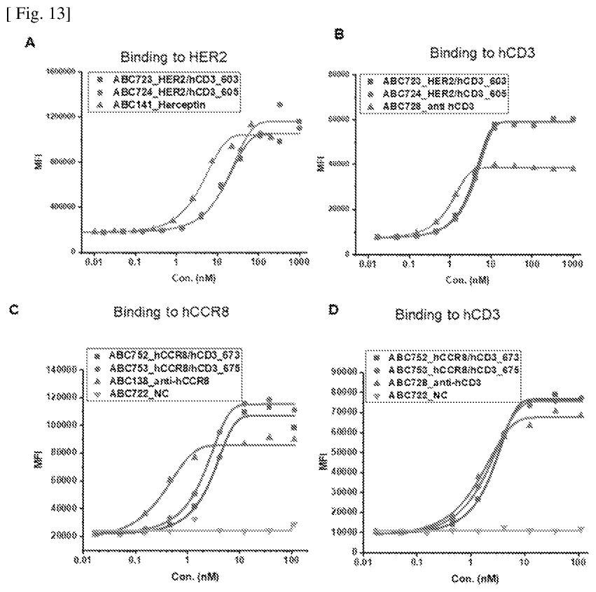

The antigen binding ability of generated bispecifics were evaluated by FACS. The EC50 values of BsAbs are summarized in Table 13. All BsAbs showed comparable binding affinities with their corresponding parental antibodies (FIG. 13), indicating the broad application of the CH1/CL replaced by the engineered CD1s/β2m domain.

TABLE 13
EC50 of BsAbs to corresponding antigens.

| | EC50 to HER2(nM) | EC50 to hCD3(nM) |
|---|---|---|
| Antibody | (SKBR3) | (Jurkat cell) |
| ABC723_HER2/hCD3_603 | 16.82 | 3.35 |
| ABC724_HER3/hCD3_605 | 18.27 | 3.75 |
| ABC141_Herceptin | 4.15 | / |
| ABC728_anti-hCD3 | / | 0.83 |

| | EC50 to hCCR8(nM) | EC50 to hCD3(nM) |
|---|---|---|
| Antibody | (CCR8-transfected 293T cell) | (Jurkat cell) |
| ABC752_hCCR8/hCD3_673 | 2.90 | 2.57 |
| ABC753_hCCR8/hCD3_675 | 2.14 | 2.09 |
| ABC138_anti-hCCR8 | 0.34 | / |
| ABC728_anti-hCD3 | / | 1.51 |

Davis, J. H., Aperlo, C., Li, Y., Kurosawa, E., Lan, Y., Lo, K. M., and Huston, J. S. (2010). SEEDbodies: fusion proteins based on strand-exchange engineered domain (SEED) CH3 heterodimers in an Fe analogue platform for asymmetric binders or immunofusions and bispecific antibodies. Protein Eng Des Sel 23, 195-202. 10. 1093/protein/gzp094.
Dillon, M., Yin, Y., Zhou, J., McCarty, L., Ellerman, D., Slaga, D., Junttila, T. T., Han, G., Sandoval, W., Ovacik, M. A., et al. (2017). Efficient production of bispecific IgG of different isotypes and species of origin in single mammalian cells. MAbs 9, 213-230. 10. 1080/19420862. 2016. 1267089.
Escobar-Cabrera, E., Lario, P., Baardsnes, J., Schrag, J., Durocher, Y., and Dixit, S. (2017). Asymmetric Fc Engineering for Bispecific Antibodies with Reduced Effector Function. Antibodies (Basel) 6. 10. 3390/antib6020007.
Gunasekaran, K., Pentony, M., Shen, M., Garrett, L., Forte, C., Woodward, A., Ng, S. B., Born, T., Retter, M., Manchulenko, K., et al. (2010). Enhancing antibody Fc heterodimer formation through electrostatic steering effects: applications to bispecific molecules and monovalent IgG. J Biol Chem 285, 19637-19646. 10. 1074/jbc. M110.117382.
Kitazawa, T., and Shima, M. (2020). Emicizumab, a humanized bispecific antibody to coagulation factors IXa and X with a factor VIIIa-cofactor activity. Int J Hematol 111, 20-30. 10. 1007/s12185-018-2545-9.
Labrijn, A. F., Meesters, J. I., de Goeij, B. E., van den Bremer, E. T., Neijssen, J., van Kampen, M. D., Strumane, K., Verploegen, S., Kundu, A., Gramer, M. J., et al. (2013). Efficient generation of stable bispecific IgG1 by controlled Fab-arm exchange. Proc Natl Acad Sci USA 110, 5145-5150. 10. 1073/pnas. 1220145110.
Lewis, S. M., Wu, X., Pustilnik, A., Sereno, A., Huang, F., Rick, H. L., Guntas, G., Leaver-Fay, A., Smith, E. M., Ho, C., et al. (2014). Generation of bispecific IgG antibodies by structure-based design of an orthogonal Fab interface. Nat Biotechnol 32, 191-198.10.1038/nbt. 2797.
Liu, Z., Leng, E. C., Gunasekaran, K., Pentony, M., Shen, M., Howard, M., Stoops, J., Manchulenko, K., Razinkov, V., Liu, H., et al. (2015). A novel antibody engineering strategy for making monovalent bispecific heterodimeric IgG antibodies by electrostatic steering mechanism. J Biol Chem 290, 7535-7562. 10. 1074/jbc. M114.620260.
Moore, G. L., Bernett, M. J., Rashid, R., Pong, E. W., Nguyen, D. T., Jacinto, J., Eivazi, A., Nisthal, A., Diaz, J. E., Chu, S. Y., et al. (2019). A robust heterodimeric Fc platform engineered for efficient development of bispecific antibodies of multiple formats. Methods 154, 38-50. 10. 1016/j. ymeth. 2018. 10. 006.
Regula, J. T., Imhof-Jung, S., Molhoj, M., Benz, J., Ehler, A., Bujotzek, A., Schaefer, W., and Klein, C. (2018). Variable heavy-variable light domain and Fab-arm CrossMabs with charged residue exchanges to enforce correct light chain assembly. Protein Eng Des Sel 31, 289-299. 10. 1093/protein/gzy021.
Ridgway, J. B., Presta, L. G., and Carter, P. (1996). ‘Knobs-into-holes’ engineering of antibody CH3 domains for heavy chain heterodimerization. Protein Eng 9, 617-621. 10. 1093/protein/9.7.617.
Spiess, C., Merchant, M., Huang, A., Zheng, Z., Yang, N.Y., Peng, J., Ellerman, D., Shatz, W., Reilly, D., Yansura, D. G., and Scheer, J. M. (2013). Bispecific antibodies with natural architecture produced by co-culture of bacteria expressing two distinct half-antibodies. Nat Biotechnol 31, 753-758. 10. 1038/nbt. 2621.
Zhao, C., Zhang, W., Gong, G., Xie, L., Wang, M. W., and Hu, Y. (2021). A new approach to produce IgG4-like bispecific antibodies. Sci Rep 11, 18630. 10. 1038/s41598-021-97393-2.

1. A new protein comprising two pairs of polypeptides, wherein each pair of polypeptides consists of a heavy chain and a light chain, the heavy chain comprises a VH domain and a CH1 domain, the light chain comprises a VL domain and a CL domain, and the VH domain and the VL domain of a first pair of polypeptides can form a first Fv fragment that specifically binds to a first antigen, the VH domain and the VL domain of a second pair of polypeptides can form a second Fv fragment that specifically binds to a second antigen, and
wherein at least one pair of dimerization domains are introduced into the first pair of polypeptides, and each pair of dimerization domains can form a dimer themselves alone or in conjunction with other proteins, wherein the dimerization domains are either homodimerization domains or heterodimerization domains.

2. The new protein of claim 1, wherein at least one pair of the heterodimerization domains can be derived from human or other mammals, and the heterodimerization domains are not TCR α/β or TCR β/α constant domains, preferably the heterodimerization domains are not TCR α/β or TCR β/α constant domains or HLA/β2m or β2m/HLA domains.

3. The new protein of claim 1, wherein at least one pair of the heterodimerization domains are selected from a pair of HLA and β2m, a pair of β2m and CD1b, a pair of CD1a and β2m, a pair of GABA1 and GABA2, or a pair of SIRPα and CD47.

4. The new protein of claim 1, wherein the heterodimerization domains are selected from at least one of the combinations of HLA/β2m, β2m/HLA, CD1b/β2m, β2m/CD1b, β2m/CD1a and CD1a/β2m.

5. The new protein of claim 1, wherein the heterodimerization domains are selected from β2m/CD1b or CD1b/β2m or β2m/CD1a or CD1a/β2m, preferably the CH1 domain of heavy chain 1 is replaced with α3 domain of CD1a and CL of light chain 1 is replaced with β2m.

6. The new protein of claim 5, wherein β2m/CD1a or CD1a/β2m, or β2m/CD1b or CD1b/β2m comprises mutations.

7. The new protein of claim 6, wherein β2m/CD1a or CD1a/β2m, or β2m/CD1b or CD1b/β2m comprise one or more disulfide bonds by introducing cysteine mutations.

8. The new protein of claim 1, wherein at least one pair of the homodimerization domains can be derived from human or other mammals, and the homodimerization domains are selected from IgG CH3/CH3 domain, IgM CH2/CH2 domain or IgE CH2/CH2 domain.

9. The new protein according to claim 1, wherein the new protein comprises four chains of polypeptides, the four chains of polypeptides are heavy chain 1, light chain 1, heavy chain 2 and light chain 2 respectively,
wherein the heavy chain 1 and the heavy chain 2 comprise a VH domain, a CH1 domain, a CH2 domain and a CH3 domain and form a long chain of VH-CH1-CH2-CH3 respectively; the light chain 1 and the light chain 2 comprise a VL domain and a CL domain and form a short chain of VL-CL respectively,
wherein the heavy chain 1 and the light chain 1 are derived from an antibody A, the single heavy chain 1 pairs with the single light chain 1 and forms half-body A; the heavy chain 2 and the light chain 2 are derived from an antibody B, the single heavy chain 2 pairs with the single light chain 2 and forms half-body B; and
wherein the CH1 domain of the heavy chain 1 pairs with the CL domain of the light chain 1, named herein as a combination of CH1/CL in half-body A, and the CH1 domain of the heavy chain 2 pairs with the CL domain of the light chain 2, named herein as a combination of CH1/CL in half-body B.

10. The new protein according to any one of claims 1-9, further containing one or more modifications, wherein the modifications can improve the correct assembly and thermal stability of the new protein.

11. The new protein of claim 10, wherein the modification comprises a knobs-into-holes.

12. The new protein of claim 9, wherein part of half-body A and/or part of half-body B is replaced by at least one pair of the homo/heterodimerization domains, and each pair of the homo/heterodimerization domains can form a homodimer or heterodimer themselves alone or in conjunction with other proteins, preferably, the CH1/CL in half-body A and/or the CH1/CL in half-body B is replaced by at least one pair of the homo/heterodimerization domains.

13. The new protein of claim 12, wherein the CH2 of the heavy chain 1 and the CH2 of the heavy chain 2 are replaced by at least one pair of the homo/heterodimerization domains.

14. The new protein of claim 12, wherein the CH3 of the heavy chain 1 and the CH3 of the heavy chain 2 are replaced by at least one pair of the homo/heterodimerization domains.

15. The new protein of claim 9, wherein at least one pair of the homo/heterodimerization domains are inserted in the new protein, and each pair of the homo/heterodimerization domains can form a homodimer or heterodimer themselves alone or in conjunction with other proteins.

16. The new protein of claim 15, wherein at least one pair of the homo/heterodimerization domains are directly inserted between the CH1/CL domains and the CH2/CH2 domains.

17. The new protein of claim 15, wherein at least one pair of the homo/heterodimerization domains are directly inserted between the CH1/CL domains and VH/VL domains.

18. The new protein of claim 15, wherein at least one pair of the homo/heterodimerization domains are directly inserted following the CH3/CH3 domains.

19. The new protein of claim 18, wherein at least one pair of the homo/heterodimerization domains are fused to the new protein, and each pair of the homo/heterodimerization domains can form a homodimer or heterodimer themselves alone or in conjunction with other proteins.

20. The new protein of claim 19, wherein at least one pair of the homo/heterodimerization domains are fused to any two chains of the new protein.

21. The new protein of claim 19, wherein at least one pair of the homo/heterodimerization domains are fused to the light chain 1 and/or the heavy chain 1.

22. The new protein of claim 19, wherein at least one pair of the homo/heterodimerization domains are fused to the heavy chain 1 and/or the heavy chain 2.

23. The new protein of claim 19, wherein at least one pair of the homo/heterodimerization domains are fused to the light chain 2 and/or the heavy chain 2.

24. The new protein of claim 19, wherein two pairs of the homo/heterodimerization domains are fused to the light chain 1 and the heavy chain 1 in half-body A, and the light chain 2 and the heavy chain 2 in half-body B, respectively.

25. The new protein of claim 9, wherein the CH1/CL in half-body A and/or the CH1/CL in half-body B is replaced by at least one pair of the homo/heterodimerization domains, and each pair of the homo/heterodimerization domains can form a homodimer or heterodimer themselves alone or in conjunction with other proteins.

26. The new protein of claim 9, wherein the CH1 domain of heavy chain 1 is replaced with CD1a^{D240C} and CL of light chain 1 is replaced with β2m^{R12C}.

27. The new protein of claim 9, wherein the CH1 domain of heavy chain 1 is replaced with CD1a^{S238C} and CL of light chain 1 is replaced with β2m^{R12C}.

28. The new protein of claim 9, wherein the CH1 domain of heavy chain 1 is replaced with CD1a^{D234C} and CL of light chain 1 is replaced with β2m_{Q8C}.

29. The new protein of claim 9, wherein the CH1 domain of heavy chain 1 is replaced with CD1a^{G194C} and CL of light chain 1 is replaced with 2m^{M99C}.

30. The new protein of claim 9, wherein the CH1 domain of heavy chain 1 is replaced with CD1a^{W190C} and CL of light chain 1 is replaced with β2m^{P14C}.

31. The new protein of claim 9, wherein the CH1 domain of heavy chain 1 is replaced with CD1a^{G194C} and CL of light chain 1 is replaced with β2m^{100C}.

32. The new protein of claim 9, wherein the CH1 domain of the heavy chain 1 is replaced with CD1a^{G194C} and the CL of the light chain 1 is replaced with β2m^{100C/Q8K}.

33. The new protein of claim 9, wherein the CH1 domain of the heavy chain 1 is replaced with CD1a^{G194C-G233S} and the CL of the light chain 1 is replaced with β2m^{100C/Q8K}.

34. The new protein of claim 9, wherein the CH1 domain of the heavy chain 1 is replaced with CD1a^{G194C-G233S/D234E} and the CL of the light chain 1 is replaced with β2 m^{100C/Q8K}.

35. The new protein of claim 9, wherein the CH1 domain of the heavy chain 1 is replaced with CD1b^{G194C} and the CL of the light chain 1 is replaced with β2m^{100C/Q8K}.

36. The new protein of claim 9, wherein the CH1 domain of the heavy chain 1 is replaced with CD1b^{G194C-G233S} and the CL of the light chain 1 is replaced with β2 m^{100C/Q8K}.

37. The new protein of claim 9, wherein the CH1 domain of the heavy chain 1 is replaced with CD1b^{G194C/G233S/D234E} and the CL of the light chain 1 is replaced with β2m^{100C/Q8K}.

38. The new protein of claim 1, which is selected from the protein ABC060, ABC061, ABC570, ABC571, ABC572, ABC132, ABC133, ABC074, ABC075, ABC131, ABC171, ABC172, ABC173, ABC174, ABC215, ABC588, ABC591, ABC270, ABC271, ABC272, ABC373, ABC374, ABC405, ABC478, ABC603, ABC672, ABC673, ABC513, ABC604, ABC605, ABC674, ABC675, ABC736, ABC737, ABC738, ABC739, ABC723, ABC724, ABC752, or ABC753,
wherein ABC060 consists of four chains shown in SEQ ID Nos: 13, 11, 2 and 4 respectively, ABC061 consists of four chains shown in SEQ ID Nos: 14, 12, 2 and 4 respectively, ABC570 consists of four chains shown in SEQ ID Nos: 82, 83, 3 and 4 respectively, ABC571 consists of four chains shown in SEQ ID Nos: 84, 83, 3 and 4 respectively, ABC572 consists of four chains shown in SEQ ID Nos: 82, 85, 3 and 4 respectively, ABC132 consists of four chains shown in SEQ ID Nos: 14, 26, 2 and 4 respectively, ABC133 consists of four chains shown in SEQ ID Nos: 27, 11, 2 and 4 respectively, ABC074 consists of four chains shown in SEQ ID Nos: 22, 21, 2 and 4 respectively, ABC075 consists of four chains shown in SEQ ID Nos: 24, 23, 2 and 4 respectively, ABC131 consists of four chains shown in SEQ ID Nos: 19, 20, 2 and 4 respectively, ABC171 consists of four chains shown in SEQ ID Nos: 14, 25, 3 and 1 respectively, ABC172 consists of four chains shown in SEQ ID Nos: 14, 26, 3 and 1 respectively, ABC173 consists of four chains shown in SEQ ID Nos: 27, 11, 3 and 1 respectively, ABC174 consists of four chains shown in SEQ ID Nos: 28, 11, 3 and 1 respectively, ABC734 consists of four chains shown in SEQ ID Nos: 13, 11, 3 and 1 respectively, ABC735 consists of four chains shown in SEQ ID Nos: 14, 12, 3 and 1 respectively, ABC215 consists of four chains shown in SEQ ID Nos: 38, 41, 45 and 44 respectively, ABC588 consists of four chains shown in SEQ ID Nos: 27, 11, 47 and 46 respectively, ABC591 consists of four chains shown in SEQ ID Nos: 36, 37, 47 and 46 respectively, ABC270 consists of four chains shown in SEQ ID Nos: 66, 65, 3 and 1 respectively, ABC271 consists of four chains shown in SEQ ID Nos: 67, 65, 3 and 1, ABC272 consists of four chains shown in SEQ ID Nos: 68, 69, 3 and 1 respectively, ABC373 consists of four chains shown in SEQ ID Nos: 73, 71, 3 and 1 respectively, ABC374 consists of four chains shown in SEQ ID Nos: 72, 70, 3 and 1 respectively, ABC405 consists of four chains shown in SEQ ID Nos: 73, 74, 3 and 1 respectively, ABC478 consists of four chains shown in SEQ ID Nos: 76, 74, 3 and 1 respectively, ABC603 consists of four chains shown in SEQ ID Nos: 76, 75, 3 and 1 respectively, ABC672 consists of four chains shown in SEQ ID Nos: 77, 75, 3 and 1 respectively, ABC673 consists of four chains shown in SEQ ID Nos: 78, 75, 3 and 1 respectively, ABC513 consists of four chains shown in SEQ ID Nos: 79, 74, 3 and 1 respectively, ABC604 consists of four chains shown in SEQ ID Nos: 80, 74, 3 and 1 respectively, ABC605 consists of four chains shown in SEQ ID Nos: 80, 75, 3 and 1 respectively, ABC674 consists of four chains shown in SEQ ID Nos: 81, 75, 3 and 1 respectively, ABC675 consists of four chains shown in SEQ ID Nos: 59, 75, 3 and 1 respectively, ABC736 consists of four chains shown in SEQ ID Nos: 86, 75, 2 and 1 respectively, ABC737 consists of four chains shown in SEQ ID Nos: 87, 75, 2 and 1 respectively, ABC738 consists of four chains shown in SEQ ID Nos: 88, 75, 2 and 1 respectively, and ABC739 consists of four chains shown in SEQ ID Nos: 89, 75, 2 and 1 respectively, ABC723 consists of four chains shown in SEQ ID Nos: 76, 75, 47 and 46 respectively, ABC724 consists of four chains shown in SEQ ID Nos: 59, 75, 47 and 46 respectively, ABC752 consists of four chains shown in SEQ ID Nos: 106, 105, 47 and 46 respectively, and ABC753 consists of four chains shown in SEQ ID Nos: 107, 105, 47 and 46 respectively.

39. The new protein of claim 9, wherein the antibody A and the antibody B can be the same or different.

40. The new protein of claim 9, wherein the antibody A or the antibody B is selected from any one of cytotoxic antibodies, inhibitors of cell proliferation, regulators of cell activation and interaction, regulators of the human immune system, and neutralizations of antigens.

41. The new protein of claim 9, wherein the antibody A or the antibody B is selected from any one of a group consisting of anti-HER2 antibody, anti-CCR8 antibody, anti-FAP antibody, anti-OX-40 antibody, anti-41BB antibody, anti-Angiopoietin-2 antibody, anti-ant-IL-4Rα antibody, anti-BCMA antibody, anti-Blys antibody, anti-BTNO2 antibody, anti-C5 antibody, anti-CD122 antibody, anti-CD13 antibody, anti-CD133 antibody, anti-CD137 antibody, anti-CD138 antibody, anti-CD16a antibody, anti-CD19 antibody, anti-CD20 antibody, anti-CD22 antibody, anti-CD27 antibody, anti-CD28 antibody, anti-CD3 antibody, anti-CD30 antibody, anti-CD33 antibody, anti-CD38 antibody, anti-CD40 antibody, anti-CD47 antibody, anti-CD-8 antibody, anti-CEA antibody, anti-CGPR/CGRPR antibody, anti-CSPGs antibody, anti-CTLA4 antibody, anti-CTLA-4 domains antibody, anti-DLL-4 antibody, anti-EGFR antibody, anti-EpCAM antibody, anti-factor IXa antibody, anti-factor×antibody, anti-GITR antibody, anti-GP130 antibody, anti-Her3 antibody, anti-HSG antibody, anti-ICOS antibody, anti-IGF1 antibody, anti-IGF1/2 antibody, anti-IGF-1R antibody, anti-IGF2 antibody, anti-IGFR antibody, anti-IL-1 antibody, anti-IL-12 antibody, anti-IL-12p40 antibody, anti-IL-13 antibody, anti-IL-17A antibody, anti-IL-1β antibody, anti-IL-23 antibody, anti-IL-5 antibody, anti-IL-6 antibody, anti-IL-6R antibody, anti-Lag-3 antibody, anti-LAG3 antibody, anti-MAG antibody, anti-Met antibody, anti-NgR antibody, anti-NogoA antibody, anti-OMGp antibody, anti-OX40 antibody, anti-PD-1 antibody, anti-PDGFR antibody, anti-PDL-1 antibody, anti-PSMA antibody, anti-RGMA antibody, anti-RGMB antibody, anti-SARS-CoV-2 antibody, anti-Te38 antibody, anti-TIM-3 antibody, anti-TNF antibody, anti-TNFα antibody, anti-TROP-2 antibody, anti-TWEAK antibody, anti-VEGF antibody, and anti-VEGFR antibody.

42. The new protein of claim 9, wherein the antibody B is anti-FAP antibody, and the antibody A is anti-HER2 antibody.

43. The new protein of claim 9, wherein the half-body B is unchanged or changed.

44. The new protein of claim 9, wherein a tag is fused to the half-body B.

45. The new protein of claim 44, wherein the tag is fused to the C terminus of light chain 2 of half-body B.

46. The new protein of claim 44, wherein the tag is used to purify the new protein or label the new protein.

47. The new protein of claim 44, wherein the tag is selected from a group consisting of SUMO tag, HIS tag, Flag tag, HA tag, MYC tag, SBP tag, CBD tag, GST tag, MBP tag, pMAL tag, IMPACT tag, Protein A and GFP.

48. The new protein of claim 44, wherein a SUMO tag is fused to C-terminal of the light chain 2 in half-body B, separated by the thrombin cleavage sequence.

49. The new protein of claim 9, wherein the half-body A is assembled to the half-body B in any form.

50. The new protein of claim 9, wherein the half-body A is assembled to the half-body B in the form of knobs-into-holes.

51. The new protein of claim 9, wherein the half-body A is assembled to half-body B by a CH3 with knobs-into-holes mutations either in heavy chain 1 or heavy chain 2.

52. An isolated polynucleotide encoding the heavy chain 1, the light chain 1, the heavy chain 2 or the light chain 2 of the new protein according to any one of claims 1-51.

53. A set of isolated polynucleotides, comprising the polynucleotide encoding the heavy chain 1, the polynucleotide encoding light chain 1, the polynucleotide encoding heavy chain 2 and the polynucleotide encoding light chain 2 of the new protein according to any one of claims 1-51.

54. An isolated vector comprising the isolated polynucleotide according to claim 52.

55. A host cell comprising the isolated polynucleotide according to claim 52 or the set of insolated polynucleotides of claim 53 or the isolated vector according to claim 54.

56. A pharmaceutical composition comprising the new protein according to any one of claims 1-51 or the isolated polynucleotide according to claim 52 or the set of isolated polynucleotides according to claim 53 or the isolated vector according to claim 54 or the host cell according to claim 55, and a pharmaceutically acceptable carrier.

57. Use of the new protein according to any one of claims 1-51 or the isolated polynucleotide according to claim 52 or the set of isolated polynucleotides according to claim 53 or the isolated vector according to claim 54 or the host cell according to claim 55 or the pharmaceutical composition according to claim 56 in the manufacture of a drug for preventing or treating a disease, or in the manufacture of a kit for diagnosing a disease.

58. A method of preventing or treating a disease in a subject in need thereof, comprising administrating to the subject a therapeutically effective amount of the new protein according to any one of claims 1-51 or the isolated polynucleotide according to claim 52 or the set of isolated polynucleotides according to claim 53 or the isolated vector according to claim 54 or the host cell according to claim 55 or the pharmaceutical composition according to claim 56.

59. A method for production of the new protein according to anyone of claims 1-51, comprising introducing an expression vector of the heavy chain 1, an expression vector of the light chain 1, an expression vector of the heavy chain 2 and an expression vector of the light chain 2 together into an expression host cell, or a combination of the expression vectors into the separate expression host cells, and expressing them under a proper condition.

60. The method of claim 59, wherein correct pairing of the new protein is insensitive to the molar ratio of expression vectors of heavy chain 1:light chain 1:heavy chain 2:light chain 2, preferably, the molar ratio of expression vectors of heavy chain 1:light chain 1:heavy chain 2:light chain 2 has no limitation, more preferably the molar ratio of expression vectors of heavy chain 1:light chain 1:heavy chain 2:light chain 2 is 1:1:1:1 or 2:2:1:1 or 1:1:2:2 or 1:3:1:1, or 1:1:1:3.

61. The method of claim 59 or 60, wherein the host cell is eukaryotic cells.

62. The method of claim 59 or 60, wherein the host cell is mammalian cells.

63. A method for production of a new protein with a substantially the same or improved thermal stability, especially with a substantially the same or improved Tm compared with an original protein, wherein the original protein has four chains of polypeptides, and the four chains of polypeptides are heavy chain 1, light chain 1, heavy chain 2 and light chain 2 respectively.
wherein the heavy chain 1 and the heavy chain 2 comprise VH domain, CH1 domain, CH2 domain and CH3 domain and form a long chain of VH-CH1-CH2-CH3, respectively; the light chain 1 and the light chain 2 comprise VL domain and CL domain and form a short chain of VL-CL, respectively;
wherein the heavy chain 1 and the light chain 1 are derived from the antibody A, the single heavy chain 1 pairs with the single light chain 1 and forms half-body A; the heavy chain 2 and the light chain 2 are derived from the antibody B, the single heavy chain 2 pairs with the single light chain 2 and forms half-body B;
wherein the CH1 domain of the heavy chain 1 pairs with the CL domain of the light chain 1, named herein as a combination of CH1/CL of half-body A, the CH1 domain of heavy chain 2 pairs with the CL domain of the light chain 2, named herein as a combination of CH1/CL of half-body B; and the method is characterized in that the combination CH1/CL of half-body A is replaced with homodimerization domains or other heterodimerization domains.

64. The method of claim 63, wherein the half-body B is unchanged or changed.

65. The method of claim 63, wherein the heterodimerization domains can be derived from human or other mammals, and the heterodimerization domains are not TCR α/β or TCR β/α constant domains, preferably the heterodimerization domains are not TCR α/β or TCR β/α constant domains or HLA/β2m or β2m/HLA domains.

66. The method of claim 63, wherein the heterodimerization domains are selected from a pair of HLA and β2m, a pair of β2m and CD1b, a pair of CD1a and β2m, a pair of GABA1 and GABA2, or a pair of SIRPα and CD47.

67. The method of claim 63, wherein the heterodimerization domains are selected from at least one of the combinations of HLA/β2m, β2m/HLA, CD1b/β2m, β2m/CD1b, β2m/CD1a and CD1a/β2m.

68. The method of claim 63, wherein the heterodimerization domains are selected from β2m/CD1b or CD1b/β2m or β2m/CD1a or CD1a/β2m, preferably, the CH1 domain of heavy chain 1 is replaced with α3 domain of CD1a and CL of light chain 1 is replaced with β2m.

69. The method of claim 63, further comprising changing the amino acid of the new protein by a mutation.

70. The method of claim 63, wherein β2m/CD1a or CD1a/2m, or β2m/CD1b or CD1b/β2m comprises mutations

71. The method of claim 63, wherein β2m/CD1a or CD1a/β2m, or β2m/CD1b or CD1b/β2m comprise one or more disulfide bonds by introducing cysteine mutations.

72. The method of claim 63, wherein the homodimerization domains can be derived from human or other mammals, and the homodimerization domains are selected from IgG CH3/CH3 domain, IgM CH2/CH2 domain or IgE CH2/CH2 domain.

73. The method of claim 63, wherein the CH1 domain of the heavy chain 1 is replaced with CD1a^{D240C} and the CL of the light chain 1 is replaced with β2m^{R12C}.

74. The method of claim 63, wherein the CH1 domain of the heavy chain 1 is replaced with CD1a^{S238C} and the CL of the light chain 1 is replaced with β2m^{R12C}.

75. The method of claim 63, wherein the CH1 domain of the heavy chain 1 is replaced with CD1a^{D234C} and the CL of the light chain 1 is replaced with β2m^{Q8C}.

76. The method of claim 63, wherein the CH1 domain of the heavy chain 1 is replaced with CD1a^{G194C} and the CL of the light chain 1 is replaced with β2m^{M99C}.

77. The method of claim 63, wherein the CH1 domain of the heavy chain 1 is replaced with CD1a^{W190C} and the CL of the light chain 1 is replaced with β2m^{P14C}.

78. The method of claim 63, wherein the CH1 domain of the heavy chain 1 is replaced with CD1a^{G194C} and the CL of the light chain 1 is replaced with β2m^{100C}.

79. The method of claim 63, wherein the CH1 domain of the heavy chain 1 is replaced with CD1a^{G194C} and the CL of the light chain 1 is replaced with β2m^{100C/Q8K}.

80. The method of claim 63, wherein the CH1 domain of the heavy chain 1 is replaced with CD1a^{G194C/G233S} and the CL of the light chain 1 is replaced with β2m^{100C/Q8K}.

81. The method of claim 63, wherein the CH1 domain of the heavy chain 1 is replaced with CD1a^{G194C/G233S/D234E} and the CL of the light chain 1 is replaced with β2 m^{100C/Q8K}.

82. The method of claim 63, wherein the CH1 domain of the heavy chain 1 is replaced with CD1b^{G194C} and the CL of the light chain 1 is replaced with β2m^{100C/Q8K}.

83. The method of claim 63, wherein the CH1 domain of the heavy chain 1 is replaced with CD1b^{G194C/G233S} and the CL of the light chain 1 is replaced with β2m^{100C/Q8K}.

84. The method of claim 63, wherein the CH1 domain of the heavy chain 1 is replaced with CD1b^{G194C/G233S/D234E} and the CL of the light chain 1 is replaced with β2m^{100C/Q8K}.

85. The method of claim 63, wherein the Tm is substantially the same or improved by at least 0.1° C., 0.2° C., 0.3° C., 0.4° C., 0.5° C., 0.6° C., 0.7° C., 0.8° C., 0.9° C., 1° C., 1.5° C., 2° C., 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., preferably at least 0.3° C., 0.5° C., 1.5° C., 2° C., or 3° C. compared with the original protein.
